(19) United States
(12) Patent Application Publication
(10) Pub. No.: US 2021/0033608 A1
(43) Pub. Date: Feb. 4, 2021

(54) Methods and Systems for Identification of Human Leukocyte Antigen Peptide Presentation and Applications Thereof

(71) Applicant: The Board of Trustees of the Leland Stanford Junior University, Stanford, CA (US)

(72) Inventors: Arash Ash Alizadeh, San Mateo, CA (US); Russ B. Altman, Menlo Park, CA (US); Binbin Chen, Stanford, CA (US); Karan Raj Kathuria, Stanford, CA (US)

(73) Assignee: The Board of Trustees of the Leland Stanford Junior University, Stanford, CA (US)

(21) Appl. No.: 16/943,951

(22) Filed: Jul. 30, 2020

Related U.S. Application Data
(60) Provisional application No. 62/880,566, filed on Jul. 30, 2019.

(51) Int. Cl. G01N 33/569; C07K 14/74

(52) U.S. Cl. CPC G01N 33/56977; G01N 33/56972; C07K 14/70539

(57) ABSTRACT

Processes and computational frameworks to determine major histocompatibility complex (MHC) presentation of peptides are described. Peptides of varying length can be queried to determine the likelihood that the peptide would be presented on MHC I or MHC II. Peptides determined to be presented can be utilized in various downstream applications.

### CROSS REFERENCE TO RELATED APPLICATIONS

This application claims priority to U.S. Provisional Application Ser. No. 62/880,566, entitled “Neural Network for Accurate Prediction of HLA Class II Antigen Presentation,” filed Jul. 30, 2019, which is incorporated herein by reference in its entirety.

### STATEMENT REGARDING FEDERALLY SPONSORED RESEARCH OR DEVELOPMENT

This invention was made with Government support under contract CA 194389 awarded by the National Institutes of Health. The Government has certain rights in the invention.

### REFERENCE TO A SEQUENCE LISTING SUBMITTED ELECTRONICALLY VIA EFS-WEB

The instant application contains a Sequence Listing which has been filed electronically in ASCII format and is herein incorporated by reference in its entirety. Said ASCII copy, created on Jul. 30, 2020, is named “06026 Seq list_ST25” and is 10,104 bytes in size.

### TECHNICAL FIELD

The invention is generally directed toward methods and systems to infer peptide presentation via major histocompatibility complexes (MHC) I and II, and more specifically directed towards methods and systems to analyze peptides of varying length for MHC I and II presentation, and various applications thereof.

### BACKGROUND

The major histocompatibility complex (MHC) is a large locus on vertebrate DNA containing a set of genes that produce cell surface proteins essential for the adaptive immune system. These cell surface proteins are referred to as human leukocyte antigen (HLA) receptors that present small protein fragments (known as peptides) to induce an immune response. There are two major classes of MHCs, MHC I and MHC II, each having a unique set of HLA receptors. MHC I is associated with the HLA-A, HLA-B, and HLA-C receptors, each of which present peptides on the cell surface. MHC II is associated with the HLA-DP, HLA-DQ, and HLA-DR receptors, each of which present peptides on the cell surface. Each HLA receptor has a number alleles that are differentially expressed between individuals of the human population. The differences of expression of the HLA alleles confers unique immunological and allergen responses for each individual, as determined by the peptides the HLA receptor presents.

### SUMMARY

Various embodiments are directed systems and methods for identification of MHC I or MHC II antigen peptides. In various embodiments, a computational framework incorporates one or more modules that are utilized to determine a MHC I or MHC II presentation score. In various embodiments, MHC presentation scores are utilized to prioritize peptides in downstream applications, including (but not limited to) peptide synthesis, vaccine development, tolerance induction, and T cell therapy.

In an embodiment, the likelihood that a peptide is presented on a human leukocyte antigen (HLA) receptor of a major histocompatibility complex (MHC) is determined. To determine the likelihood that a peptide is presented, one or more peptides sequences for query is obtained. Each queried peptide has a length between 8 and 26 amino acids. A trained peptide presentation module incorporating a recurrent neural network architecture is obtained. The peptide presentation module is capable of determining presentation of peptides having varying length to at least one HLA allele. The one or more peptide sequences is queried one or more peptide sequences. Based on the peptide sequence and the at least one HLA allele assessed, a MHC presentation score for each peptide of the one or more peptide sequences is determined.

In another embodiment, the peptide presentation module is trained utilizing in vivo data derived from human individuals or cell lines that have had their MHC peptide ligand sequences identified by antigen presentation profiling via mass spectrometry.

In yet another embodiment, the peptide presentation module's recurrent neural network has one of the following architectures: fully recurrent, long short-term memory, gated recurrent unit, bidirectional LSTM or hierarchical recurrent network.

In a further embodiment, at least a first peptide sequence and a second peptide sequence are obtained, wherein each of the peptide length of the first peptide is different from the length of the second peptide.

In still yet another embodiment, a trained binding affinity module incorporating a recurrent neural network architecture is obtained. The binding affinity module is capable of determining binding affinity of peptides having varying length to a particular HLA allele. The trained binding affinity module is integrated with the trained peptide presentation module. The one or more peptide sequences is queried utilizing the trained binding affinity module to determine a binding affinity score between each peptide of the one or more peptide sequences and the at least one HLA allele assessed. Based on the peptide sequence, the at least one HLA allele assessed, and the binding affinity score, a MHC presentation score for each peptide of the one or more peptide sequences is determined.

In yet a further embodiment, the binding affinity module is trained utilizing in vitro data derived from the Immune Epitope Database.

In an even further embodiment, the binding affinity module's recurrent neural network has one of the following architectures: fully recurrent, long short-term memory, gated recurrent unit, bidirectional LSTM or hierarchical recurrent network.

In yet an even further embodiment, the flanking amino acid sequences upstream and downstream is determined for each peptide of the one or more peptide sequences. A trained cleavability module incorporating a neural network architecture is obtained. The trained cleavability module is capable of determining the cleavability of peptides based on their flanking amino acids. The trained cleavability module is integrated with the trained peptide presentation module. The one or more peptide sequences is queried utilizing the trained cleavability module to determine a cleavability score for each peptide of the one or more peptide sequences. Based on the peptide sequence, the at least one HLA allele assessed, and the cleavability score, a MHC presentation score for each peptide of the one or more peptide sequences is determined.

In still yet an even further embodiment, the flanking amino acids are determined from a proteome database.

In still yet an even further embodiment, the cleavability module is trained utilizing a ligandome of an antigen presenting cell line.

In still yet an even further embodiment, the gene information for each peptide of the one or more peptide sequences is obtained. A gene expression module incorporating a neural network architecture is obtained. The gene expression module is capable of determining the relative gene expression of peptides based on their gene information. The gene expression module is integrated with the trained peptide presentation module. The one or more peptide sequences is queried utilizing the trained gene expression module to determine the relative expression level for each peptide of the one or more peptide sequences. Based on the peptide sequence, the at least one HLA allele assessed, and the relative gene expression, a MHC presentation score for each peptide of the one or more peptide sequences is determined.

In still yet an even further embodiment, the gene expression module determines relative gene expression empirically from personalized RNA sequencing data.

In still yet an even further embodiment, the gene expression module determines relative gene expression inferentially from external RNA sequencing data.

In still yet an even further embodiment, the gene expression module corrects for low gene expression of extracellular proteins or blood proteins constituents.

In still yet an even further embodiment, the MHC presentation score is for MHC I. The binding affinity module is capable of determining binding affinity of peptides having a length between 8 and 17 amino acids. And the at least one HLA allele is an allele of one of: HLA-A, HLA-B, and HLA-C.

In still yet an even further embodiment, the at least one HLA allele is all alleles of HLA-A, HLA-B, and HLA-C.

In still yet an even further embodiment, the MHC presentation score is for MHC II. The binding affinity module is capable of determining binding affinity of peptides having a length between 8 and 26 amino acids. And the at least one HLA allele is an allele of one of: HLA-DP, HLA-DQ, and HLA-DR.

In still yet an even further embodiment, the at least one HLA allele is all alleles of HLA-DP, HLA-DQ, and HLA-DR.

In still yet an even further embodiment, the MHC presentation score is a basis for utilizing at least one peptide of the one or more peptide sequences in a downstream application.

In still yet an even further embodiment, the downstream application is one of: synthesizing the at least one peptide; developing a vaccine for cancer or an infectious pathogen utilizing the at least one peptide; developing a treatment to induce tolerance to the at least one peptide, wherein the peptide is involved with an autoimmune or allergic response; or developing a T cell therapy to treat cancer based on the at least one peptide.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The description and claims will be more fully understood with reference to the following figures and data graphs, which are presented as exemplary embodiments of the invention and should not be construed as a complete recitation of the scope of the invention.

FIG. 1 provides a flow diagram of a process to determine MHC I or MHC II presentation score for a peptide in accordance with various embodiments.

FIG. 2 provides comparison of the number of unique HLA-DR ligands identified within two antigen presentation profiling studies, each exceeding all HLA-DR in vitro quantitative binding measurements from previous studies within the IEDB (as of December 2018), utilized in accordance with various embodiments.

FIG. 3 provides a table of performance of NetMHCIIpan to predict mantle cell lymphoma (MCL) presented HLA-DR ligands, utilized in accordance with various embodiments.

FIG. 4 provides performance of NetMHCIIpan for discrimination of decoys from bona fide HLA-II ligands recovered by antigen presentation profiling, utilized in accordance with various embodiments. For each patient, NetMHCIIpan-predicted affinities and ranks were separately evaluated (x axis), and performance measured by ROC-AUC (y axis, dotted lines represent the median). NetMHCIIpan ranks (mean AUC=0.68) slightly outperformed NetMHCIIpan binding affinities (mean AUC=0.65, n=18; two-tailed paired t test, P=0.003).

FIG. 5 provides limited sensitivity of NetMHCIIpan for classification of HLA-DR ligands, utilized in accordance with various embodiments. Depicted is the distribution of NetMHCIIpan ranks for all 6,063 peptides identified from the JeKo-1 cell line, where 22% of HLA-II ligands identified by MS had predicted values worse than the recommended NetMHCIIpan rank cut-off for binding (10%). And in vitro binding assay results for HLA-II peptide ligands identified by MS but predicted by NetMHCIIpan not to bind HLA-II. Among ten such peptides predicted by NetMHCIIpan not to bind, nine were nevertheless confirmed to bind cognate HLA-DR alleles (04:03 and/or 07:01) by two independent flow cytometry experiments. Scatter plots depict binding of two exemplar FITC-conjugated peptides (x axis) to APC-conjugated HLA-DR proteins (y axis). Sequences listed in FIG. 5 include SEQ. ID Nos. 1-10.

FIGS. 6 and 7 provides in vitro HLA-DR peptide binding assay for experimental validation and associated results, utilized in accordance with various embodiments. Sequences listed in FIG. 6 include SEQ. ID Nos. 1-7, 9, 11, and 12. Sequences listed in FIG. 7 include SEQ. ID Nos. 5, 6, and 13-25.

FIG. 8 provides training and evaluation scheme of MARIA, as a new machine learning framework for more accurate prediction of HLA-II ligands, utilized in accordance with various embodiments. Positive examples are HLA-II ligand peptide sequences directly identified by antigen presentation profiling of human cells and tissues by immunoprecipitation (i.p.) and MS, and negative examples are length-matched random human peptides (decoys). The model separately considers binding affinities estimated using in vitro binding data. Patient HLA-II allele or genotype and gene expression information are obtained from next-generation sequencing. A RNN integrates information and produces a predictor for HLA-II ligand presentation by minimizing training errors. Independent test sets determine the final performance of the model.

FIG. 9 provides an example of how variable length amino acid sequences (8-26AA) to be one-hot encoded for machine learning purposes, utilized in accordance with various embodiments. A peptide is represented by a 21×26 matrix. Each row represents 21 possible amino acids, and each column represents the true amino acid at that position (1=true). Any positions not encoding for an amino acid due to short length of a peptide are encoded as an all-zero vector which will be ignored by the neural network masking layer.

FIGS. 10A and 10B provide detailed individual neural network architectures, utilized in accordance with various embodiments.

FIG. 11 provides a table of performance of models with all possible features combinations for predicting MCL presented HLA-DR ligands, utilized in accordance with various embodiments.

FIG. 12 provides comparison of gene expression levels of HLA-DR ligands and non-ligands, utilized in accordance with various embodiments. Gene expression was estimated by RNA-seq for HLA-DR-presented genes, all protein-coding genes and non-presented protein-coding genes, respectively. HLA-DR ligand genes have significantly higher gene expression levels than the set of all protein-coding genes (n=34,049, 23,165 and 19,464, respectively; **P<1×10-5, Mann-Whitney U test). Some HLA-DR ligands (8.4%) had undetectable levels of expression; those in this set were enriched for extracellular protein (GO enrichment; Fisher's exact test, P<1×10-17). Violin curves represent the probability distribution function of gene expression, black boxes represent middle two quartiles and white dots represent the median.

FIGS. 13 to 15 provide data showing the relationship between gene expression levels and HLA ligand presentation, utilized in accordance with various embodiments.

FIG. 16 provides cleavage signature analysis for HLA-DR ligands, utilized in accordance with various embodiments. Frequencies of 20 amino acids at 6 positions upstream (−6 to −1) and downstream (+1 to +6) of HLA-DR ligands (n=12,150) are compared to the background distribution (n=23,218) to determine amino acid enrichment and depletion surrounding HLA-DR ligands. Colors of the heat map and sizes of the logo plot letters indicate fold change. The logo plot only includes statistically significant enrichment (P<0.001, two-tailed independent t test by IceLogo). The minus symbol in the top row of the heat map indicates presented peptides that are located at the beginning or end of source protein sequences.

FIGS. 17A to 17C provide analysis of HLA ligand cleavage signatures for various cell lines, utilized in accordance with various embodiments.

FIG. 18 provides workflow of MARIA for predicting HLA-DR ligand presentation score in accordance with an embodiment. Two separate models first calculate HLA-DR peptide binding scores and peptide cleavage scores. The neural network further integrates peptide sequence and estimated gene expression level with two scores, via a recurrent layer and merge layers, to generate a presentation score indicating likelihood of HLA-II presentation.

FIG. 19 provides distributions of minimum additive distances of validation peptide sequences to training peptide sequences, utilized in accordance with various embodiments.

FIG. 20 provides performance of MARIA and four alternative predictors on 10% of the held-out validation set (true MCL HLA-II ligands, n=3,300; random human decoy peptides, n=10,000), generated in accordance with various embodiments. MARIA scores incorporating gene expression levels, peptide sequence, binding scores and cleavage scores outperformed methods using each of these features individually (DeLong test, P<1×10-5; AUC=0.92).

FIG. 21 provides detailed 10-fold cross validation performance on identifying naturally presented with different predictors, generated in accordance with various embodiments. MARIA models considering all relevant features (peptide sequence, gene expression, predicted in vitro binding, and cleavage scores) have higher average AUC scores than the second best model (RNN with sequence only, Mann-Whitney U test p<1e-5, n=10).

FIG. 22 provides validation performance of logistical regression models combining gene expression, binding scores and cleavage scores, generated in accordance with various embodiments. Logistical regression models were trained on training MCL HLA-DR ligand data, and the validation performance was reported as average AUCs of 10-fold cross validation. Combining gene expression, binding scores and cleavage scores moderately increases the AUC compared to gene expression alone or combined with one additional feature (AUC=0.82, DeLong test p<0.0001, n=3300 for ligand peptides and n=10,000 for decoy peptides).

FIG. 23 provides comparison of model precision and specificity across a range of presented MCL HLA-DR peptide prevalences, generated in accordance with various embodiments. Sensitivity for each model was controlled at 30% for all calculations, with corresponding specificity denoted adjacent to inset legend. The shaded areas represent the 95% confidence interval around the mean value, on the basis of tenfold cross-validation.

FIG. 24 provides comparison of precision and recall for different models for predicting HLA-DR ligands using various types of training data, generated in accordance with various embodiments. Precision was calculated assuming 1% prevalence of presented HLA-DR ligands. The shaded areas represent 95% confidence interval around the mean value (line), based on tenfold cross-validation.

FIGS. 25A and 25B provide comparisons of peptide presentation between HLA-DRB1*01:01 and HLA-DRB1*04:04 alleles, generated in accordance with various embodiments.

FIG. 26 provides overlap and sequence motifs of two HLA-DR ligand sets identified from two monoallelic K562 cell lines, generated in accordance with various embodiments. A proportion (31%) of peptides appeared in both the HLA-DRB1*01:01 (n=2,430) and HLA-DRB1*04:04 (n=2,072) ligand sets when considering substring matches. The sequence motifs with highest statistical significance (P<1×10-7, multiple hypergeometric test implemented by MEME) are shown.

FIG. 27 provides performance of MARIA and six alternative methods when differentiating 1,361 K562 HLA-DRB1*01:01 ligands from 1,361 human decoys, generated in accordance with various embodiments. MARIA outperformed the second best method (SMM Align; DeLong test, P<1×10-5). Limited by the IEDB Concensus3 package, only ligand sequences amino acids are included in this comparison.

FIG. 28 provides performance of MARIA and four alternative methods differentiating 2,032 K562 DRB1*04:04 ligands from 2,032 human decoys, generated in accordance with various embodiments. MARIA achieved an AUC of 0.89 AUC as compared to an AUC of 0.56 for NetMHCIIpan. RNN and SNN trained on MCL ligands obtained AUC values of 0.83 and 0.78, respectively.

FIG. 29 provides overlap and sequence motifs of two HLA-DQ ligand sets, generated in accordance with various embodiments. A majority (65%) of peptides were present in both HLA-DQ2.2 (n=7,374) and HLA-DQ2.5 (n=4,249) ligand sets when considering substring matches. The sequence motifs with highest statistical significance (P<1×10-7, multiple hypergeometric test implemented by MEME) are shown.

FIG. 30 provides overlap of HLA-DQ2.2 and HLA-DQ2.5 peptide ligands, generated in accordance with various embodiments. Ligands from these two alleles overlap 29% when counting identical peptide sequences only.

FIG. 31 provides training, validation, test of MARIA models for HLA-DQ2.2 presentation, generated in accordance with various embodiments. To train the MARIA DQ2.2 model, 5845 peptides shared between HLA-DQ2.2 and HLA-DQ2.5, and 2529 peptide unique to HLA-DQ2.2 were used as the positive examples; 8374 length-matched peptides were used as negative examples. Peptide sequences were assigned into training, validation, and test set. No peptides in validation and test set were substring of a training peptide, vice versa.

FIG. 32 provides performance of MARIA trained on HLA-DQ2.2 ligand sequences and tested on a held-out human HLA-DQ2.2 peptide set (n=650), generated in accordance with various embodiments. MARIA was trained on 90% of the HLA-DQ2.2-associated peptide sequences. MARIA achieves an AUC of 0.89 when differentiating DQ2.2 ligands from length-matched decoys. By comparison, NetMHCIIpan percentiles obtained an AUC of 0.68. Dashed red lines indicate the 90th percentile, the default cut-off for NetMHCIIpan.

FIG. 33 provides performance of MARIA and NetMHCIIpan when identifying immunogenic gluten peptide fragments (n=69), generated in accordance with various embodiments. MARIA trained on human DQ2.2 ligands identified 49% of HLA-DQ2.2-binding gluten peptides with 92% specificity. By comparison, NetMHCIIpan had 6% sensitivity and 88% specificity. Dashed red lines indicate the 90th percentile, the default cut-off for NetMHCIIpan.

FIG. 34 provides MARIA predicted presentation scores on HLA-DQ2.2 presentation of five known celiac disease related gluten peptides upon all possible Q->E or Q->K mutations, generated in accordance with various embodiments. Based on MARIA-DQ ranks, deamination forms (Q->E) of gluten peptides present better compared to unmodified forms or Q->K forms of gluten peptides (* indicates p=3e-4, **=p<1e-5, Mann-Whitney U test, n=15, 255, 7, 31, 63).

FIG. 35 provides a table of sequences and references for HLA-DP ligand validation, utilized in accordance with various embodiments. Sequences listed in FIG. 35 include SEQ. ID Nos. 26-45.

FIG. 36 provides impact of training dataset size on prediction performance for pan-HLA-II MARIA models and performance of pan-HLA-II models for differentiating HLA-DP ligands from random human peptides, generated in accordance with various embodiments.

FIG. 37 provides correlation of MARIA-predicted and experimentally identified HLA-DR-presented immunoglobulin antigens, generated in accordance with various embodiments. Eighteen MCL immunoglobulin sequences were analyzed by a version of MARIA trained on non-immunoglobulin HLA-DR ligands to determine the presentation hotspots (left, blue). The same 18 MCL samples were profiled with LC-MS/MS to determine the regions of immunoglobulin presented by HLA-DR (right, orange). Predicted and observed presentation hotspots were significantly correlated on both heavy chains and light chains (Spearman's p of 0.63 and 0.55, P=1×10-65 and 7.5×10-19; n=1,015 and 311, respectively). MARIA identified HLA-DR presentation hotspots in the immunoglobulin heavy chain variable region (IGHV). MARIA-predicted HLA-DR-presented peptides from IGHV FR3 regions more than the other six regions across patients (P<1×10-5, Mann-Whitney U test), consistent with MS findings (P<1×10-5, Mann-Whitney U test). Each dot represents predicted or experimentally identified ligand coverage in a 15-amino-acid sliding window on the aligned IGHV sequence (n=38 for the FR3 region and n=87 for the non-FR3 regions). MARIA-predicted ligand numbers were normalized with the MS-identified maximum ligand numbers for visualization purposes.

FIGS. 38 and 39 provide validating MARIA performance for predicting patient IgH HLA-DR presentation and immune response, generated in accordance with various embodiments. Sequences listed in FIG. 39 include SEQ. ID Nos. 46-49.

FIG. 40 provides comparison and structural analysis of MARIA and NetMHCIIpan for mutated CLIP peptides, generated in accordance with various embodiments.

FIG. 41 provides performance of MARIA on an independent melanoma HLA-II ligand set, generated in accordance with various embodiments. MARIA trained on MCL ligands achieved an AUC of 0.89 when differentiating patient melanoma HLA-II peptides from length-matched decoys, as compared to NetMHCIIpan with an AUC of 0.64. Shuffling correct training labels diminished the prediction performance of MARIA, reducing its AUC to 0.53. On right, curves depict the comparison of the precision (y-axis) of each of three methods (full MARIA model, NetMHCIIpan 3.1, and a “random” MARIA model trained on shuffled data) when considering a range of recall/sensitivity thresholds (x-axis). At 20% recall, MARIA achieved 38% precision (PPV), assuming a 1% prevalence of true antigen presentation.

FIG. 42 provides neoantigen gene expression in patients with melanoma is not associated with postvaccination CD4+ T cell responses, utilized in accordance with various embodiments. Personalized gene expression values were obtained from tumor RNA-seq in two personalized melanoma vaccine trials. In both trials, there is no difference in gene expression values between positive and negative vaccine candidates for their ex vivo CD4 cytokine release tests (n=127 and 97; P=0.49 and 0.50, two-tailed unpaired t test). NS, not significant. Post-vaccination CD4+ T cell responses are associated with MARIA scores. Peptide sequences from the same two clinical trials were scored with MARIA. Each candidate was stratified into three categories on the basis of the highest MARIA percentile scores among 15-amino-acid oligomer sliding windows: low (<95th), medium (95-99.5th) and high (>99.5th). Dashed red lines indicate average response rates of the whole cohort.

FIG. 43 provides performance of MARIA in predicting CD4 T-cell responses to personalized vaccines, generated in accordance with various embodiments. Plots depict results for two melanoma clinical trials of personalized cancer vaccines, where a range of MARIA score cutoffs (x-axis) are related to the Positive predictive values (PPV), negative predictive values (NPV) and sensitivity (y-axis) for predicting post-vaccination CD4 T-cell responses.

FIG. 44 provides potential CD4 T cell epitopes in a cohort based on MARIA scores and weak association of NetMHCIIpan and CD4 T-cell post-vaccination responses, generated in accordance with various embodiments. Numbers of neoantigens in melanoma above MARIA-high cut-off. Each nonsynonymous mutation in 6 melanoma patients was scored with MARIA on a basis of 15mer sliding windows. The best MARIA score of all potential 15mer windows was used to represent the neoantigen. ˜7% of nonsynonymous mutations reached 99.5% MARIA-high cut-off. Except the patient 1, all patients had at least 20 neoantigens in the MARIA-high category (MARIA percentile >99.5th). Each vaccine peptide sequence in Ott et al. was scored with NetMHCIIpan and was stratified into three categories based on the same cut-off used for MARIA: low (<95th), medium (95-99.5th) and high (>99.5th). NetMHCIIpan score categories were weakly associated with CD4 T-cell responses but did not reach statistical significance (chi-square test, p=0.3). Dashed red lines indicate average response rates of the whole cohort.

FIG. 45 provides relationship between MARIA percentile scores and CD4+ T cell responses to tumor-associated antigens across cancer types and studies, generated in accordance with various embodiments. Each of these 521 peptides (dots) were tested by MARIA, allowing comparison of percentile scores (x axis, right) with immunogenicity (blue, immunogenic; green, non-immunogenic). As depicted by the summarized inset table, 74% of immunogenic peptides (20 of 27, blue) scored above the 95th MARIA percentile threshold. Teff, effector T cells.

FIG. 46 provides relative abundance of HLA-II expression in various tumors, utilized in accordance with various embodiments.

FIG. 47 provides detailed HLA-II ligand data and gene expression data used in training and validation of MARIA models, utilized in accordance with various embodiments.

FIG. 48 provides MARIA-1 Model design in accordance with an embodiment of the invention. Each step of prediction relies on context from the antigen processing pathway. Given the peptide and MHC alleles, a model generates predicted six binding affinities. These affinities are combined with separately calculated gene expression, peptide cleavage, and MHC-peptide binding scores. This information is integrated with the peptide sequence via a recurrent layer and merge layers to generate the MARIA-I presentation score, which is the likelihood of MHC-I presentation.

FIG. 49 provides gene expression analysis for MCL MHC-I, MHC-II, all presented, and non-presented ligands, utilized in accordance with various embodiments. Gene expression levels were estimated using RNA sequencing. MHC-II peptides presented on HLA-DR have the highest gene expression, followed by MHC-I, all presented peptides, and all non-presented peptides.

FIG. 50 provides cleavage signature analysis for MCL MHC-I ligands across all alleles, utilized in accordance with various embodiments. Relative fold change for 20 amino acids for 6 amino acid upstream (−6 to −1) and downstream (1 to 6) flanking regions determine enriched and depleted patterns in MS-identified peptides versus randomly generated decoys.

FIG. 51 provides Levenshtein distances between cross-validation peptides and training peptides, utilized in various embodiments. Minimum edit distance shows that 40% of validation peptides are at least 3 amino acid changes away from an equivalent training peptide, ensuring a wide separation in peptide sequences between training and validation peptide data.

FIG. 52 provides performance of MARIA-I with 190 k and 50 k training data size and four individual feature models on 10% of the held-out validation set of MCL peptides. Performance measured using AUROC, generated in accordance with various embodiments.

FIG. 53 provides performance of MARIA-I binding sub-model compared to netMHCpan3.0 on 25,824 presented validation ligands presented by Human B lymphoblastoid cell lines engineered to express a single MHC-I allele and 25,824 decoy peptides, generated in accordance with various embodiments.

FIG. 54 provides performance of MARIA-I and three other methods on peptides presented on SK-OV-3, an ovarian cancer cell line, with 1,377 MS-identified ligands and 4,131 decoy ligands, generated in accordance with various embodiments. Performance measured using PPV at 1% prevalence and 40% sensitivity.

FIG. 55 provides performance of MARIA-I and three other methods on peptides presented on 12 meningioma samples with 45,371 MS-identified ligands and 40,916 decoy ligands, generated in accordance with various embodiments. Performance measured using PPV at 1% prevalence and 40% sensitivity.

FIG. 56 provides performance of MARIA-I and two other methods on peptides presented on 17 tumor samples with 163,128 MS-identified ligands and 489,384 decoy ligands, generated in accordance with various embodiments. Performance measured using PPV at 1% prevalence and 40% sensitivity.

FIG. 57 provides performance of murine MARIA-I haplotype d model and netMHCpan4.0 on 512 MS-identified ligands and 5,769 decoys, generated in accordance with various embodiments.

FIG. 58 provides performance of murine MARIA haplotype I-Ab model and netMHCIIpan3.2 on 1,984 MS-identified ligands and 8,376 decoys, generated in accordance with various embodiments.

FIG. 59 provides a schematic of neoantigen candidate selection using MARIA-! in accordance with an embodiment. The patient's cancer is biopsied and sequenced through whole exome sequencing. Variant calling output files are fed into an annovar pipeline that converts nonsynonymous DNA mutations into corresponding changes in amino acid sequences. Each mutation resulting in a coding change is represented as an individual peptide and processed through MARIA-I using the patient's HLA alleles. Resulting MARIA-I percentile scores are used to rank peptides, select candidate neoantigens, and validate with published ex-vivo CD8+ T cell stimulation experiments.

FIGS. 60 and 61 provide CD8+ T cell responses from TILs pulsed ex-vivo with patient-derived melanoma (FIG. 60) and gastrointestinal (FIG. 61) peptides are associated with MARIA-I scores, generated in accordance with various embodiments. WES data from publicly available data was evaluated using pipeline represented by schematic in as shown in FIG. 59 using three MARIA-I percentile cutoffs (Low:<95, Medium:95-99.5, High:>99.5).

FIG. 62 provides percent of patients' peptides scored as “highly presented” (>99.5^{th} percentile) by MARIA-I, generated in accordance with various embodiments. All patients mutations were processed using the pipeline in FIG. 59 and binned into three MARIA-I score categories. The red section of each bar indicates how many of the predicted highly presented peptides were used to vaccinate that patient. The blue section indicates the number of unused MHC-I neoantigen candidates with MARIA-I percentile scores >99.5.

### DETAILED DESCRIPTION

Turning now to the drawings and data, embodiments related to utilizing computational frameworks to identify peptides to be presented via major histocompatibility complexes (MHC) I and II as determined by the peptide sequence binding affinity, peptide cleavability, and/or peptide expression level are described herein. Accordingly, various embodiments are directed towards computational frameworks that utilize one or more modules to produce a peptide presentation score for MHC I or MHC II. In some embodiments, a computational framework includes a module to assess a peptide's binding affinity with particular human leukocyte antigen (HLA) allele to determine a likelihood of presentation via MHC I or MHC II. In some embodiments, a computational framework includes a module to assess a peptide's likelihood to be cleaved to determine a likelihood of for presentation via MHC I or MHC II. In some embodiments, a computational framework includes a module to assess a peptide's expression level to determine the likelihood of presentation via MHC I or MHC II.

Various embodiments of computational frameworks include various combinations of modules. Accordingly, in some embodiments, a computational framework utilizes one solitary module to provide a peptide presentation score. In some embodiments, a computational framework integrates two or more modules to provide a peptide presentation score.

Numerous embodiments utilize a peptide presentation score for various analyses and downstream applications. In some embodiments, numerous peptides are assessed for their likelihood to be presented via MHC I or MHC II and then compared on the basis of their peptide presentation score. In some embodiments, peptides with presentation scores over a threshold are utilized in downstream applications. In some embodiments, peptides with higher presentation scores, as compared with other peptides assessed, are utilized in downstream applications. In some embodiments, peptides of a top percentile or a top quantile of presentation scores are utilized in downstream applications.

Numerous downstream applications can be performed on the basis of a peptide having a particular presentation score. As MHC I and II peptide presentation relates to immunological activity, various peptides can be selected on the basis of its presentation score, synthesized, and then utilized in immunological applications. In some embodiments, selected peptides are synthesized and assessed for their ability to be presented via MHC I and II, confirming the computational score. In some embodiments, selected peptides are synthesized and utilized within a vaccine to induce immune responses to the peptide. Accordingly, vaccines can be created for various infectious agents and/or cancers. In some embodiments, selected peptides can be utilized to identify potential targets for T-cell therapies. In some embodiments, selected peptides are synthesized and utilized to induce a tolerance in individuals to the peptide, which can be useful in various autoimmune and allergic disorders. For example, selected wheat gluten peptides can be utilized for tolerance induction in individuals suffering from celiac disease.

### Computational Framework for Determining MHC I or MHC II Peptide Presentation

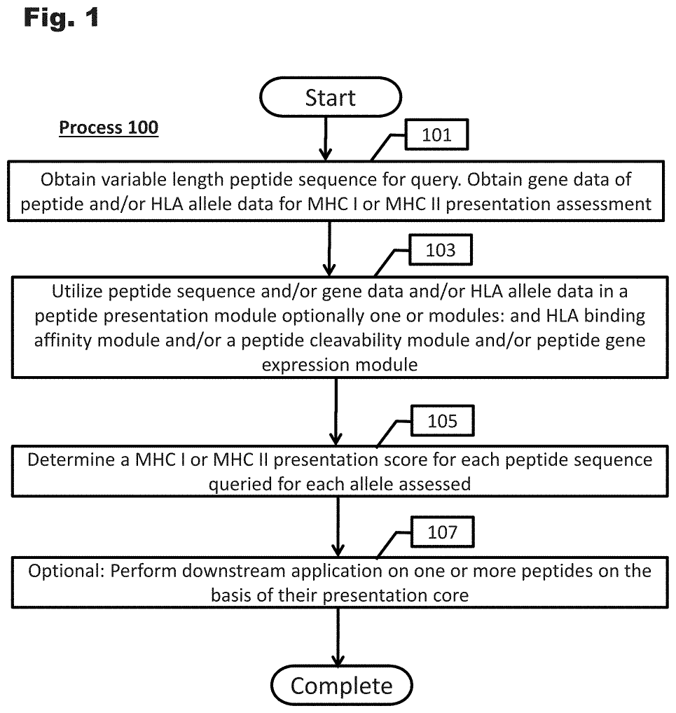

Provided in FIG. 1 is a process utilizing a computational framework to determine a peptide presentation score indicating the likelihood that the peptide would be presented by MHC I or MHC II in a biological cell, especially an antigen presenting cell. In various embodiments, one or more peptide sequences of various lengths are queried within one or more computational modules of the framework, resulting in a MHC I or MHC II presentation score. In various embodiments, one or more peptides are utilized in various downstream applications on the basis of their MHC I or MHC II presentation score.

In some embodiments, a computational framework is designed work for one of: MHC I or MHC II. In some embodiments, a computational framework is designed to consider peptide presentation by alleles of one or more HLA receptors. Particular HLA gene products work in within MHC I and particular HLA gene products work in within MHC II. In humans, the HLA gene products capable of working with MHC I are HLA-A, HLA-B, and HLA-C, each having two number of alleles with unique peptide binding propensities. Likewise, the human HLA gene products capable of working with MHC II are HLA-DP, HLA-DQ, and HLA-DR, each having a number of alleles with unique peptide binding propensities. Accordingly, various embodiments of computational frameworks and/or modules within a framework can be specific for assessment of peptides for particular HLA allele, which may be useful for analysis of human population expressing that particular allele. And in various embodiments, a computational framework and/or modules within a framework can be generalized for a plurality HLA alleles, which may be useful a more generalized population analysis. For instance, in some embodiments, a computational framework determines peptide presentation for the alleles of a single HLA receptor. Accordingly, in various embodiments, a computational framework determines peptide presentation for alleles of HLA-A, HLA-B, HLA-C, HLA-DP, HLA-DQ, or HLA-DR. And for instance, in some embodiments, a computational model determines peptide presentation for all alleles of MHC I or MHC II. Accordingly, in various embodiments, a computational framework determines peptide presentation for the alleles of HLA-A, HLA-B and HLA-C (i.e., the alleles of MHC-I) or for the alleles of HLA-DP, HLA-DQ, and HLA-DR (i.e., the alleles of MHC-II).

Process 100 can begin with obtaining (101) variable length peptide for query. In addition, and dependent on the various modules utilized for analysis, gene data of the peptide and/or HLA allele data for MHC I or MHC II presentation assessment is obtained. The length of peptides to be assessed can vary, and is dependent on MHC I or MHC II presentation. In embodiments for MHC I assessment, peptides between 8 and 17 amino acids can be assessed within the same framework. In embodiments for MHC II assessment, peptides between 8 and 26 amino acids can be assessed within the same framework. The ability to assess multiple lengths of peptides is made possible with the use of a recurrent neural network (RNN), which is discussed in more detail below and within the Exemplary embodiments.

Generally, any peptide sequence within the length requirements can be queried. It may be desirable, however, to assess particular sets of sequences. In some embodiments, endogenously expressed peptides are queried. In some embodiments, cancer neoantigens are queried, which may be useful to identify immunogenic peptides for cancer vaccines or cancer T cell treatments. In some embodiments, autoantigens are queried, which may be useful to develop treatments for autoimmune diseases. In some embodiments, exogenous peptides are queried. In some embodiments, peptides of infectious pathogens (e.g., bacteria, viruses, parasites) are queried, which may be useful to develop vaccines against these pathogens. In some embodiments, exogenous antigens involved with autoimmune disorders (e.g., wheat gluten in celiac disease) and allergies are assessed, which may be useful to develop treatments for autoimmune disorders and allergies.

In addition to peptide sequence, gene data of the peptide and/or HLA allele data may be obtained, as dependent on the modules utilized in a computational framework. As described in greater detail below, a framework includes a peptide presentation module may optionally also include a binding affinity module, a cleavability module, and/or an expression level module. A peptide presentation module utilizes a peptide sequence and HLA allele data to determine the likelihood of a peptide sequence to be presented on the HLA receptor. A binding affinity module utilizes a peptide sequence and HLA allele data to determine the ability of the HLA to bind the peptide sequence. A cleavability module utilizes the sequences flanking upstream and downstream of the peptide to determine the likelihood of a peptide to be cleaved for antigen presentation. Flanking sequences may be derived from any appropriate source that provides such data, such as (for example), gene sequence data which can be utilized to determine the sequence flanking the peptide. An expression module utilizes the peptide gene data to determine the relative expression level of the peptide.

Process 100 utilizes (103) the peptide sequence, gene data, and/or HLA allele data in a peptide presentation module and optionally one or more computational modules. Modules for peptide presentation, binding affinity, cleavability, and expression level are described below.

### Peptide Presentation Module

In numerous embodiments, a peptide presentation module computes the likelihood of a peptide to be presented by MHC I or MHC II based on its sequence, which is dependent on the HLA allele expressed. Accordingly, in some embodiments, a peptide presentation module assesses the peptide's sequence to determine if the sequence has is likely to be presented on a HLA allele (or set of HLA alleles). To determine presentation, in accordance with some embodiments, the module utilizes a recurrent neural network (RNN) that has been trained utilizing in vivo HLA allele/peptide ligand data. Training data may be obtained from patients and/or cell lines. In some embodiments, mass spec data derived from human individuals and/or cell lines to profile MHC-bound peptide ligands bound to an HLA allele. Accordingly, in some embodiments, numerous (e.g., tens of thousands) non-redundant peptide ligand sequences in association with an HLA allele are utilized as positive examples to train the binding affinity module. In some embodiments, randomly selected length matched human peptide sequences are utilized as negative examples to train the binding affinity module. Accordingly, a peptide presentation module learns the patterns of sequences that are presented from the training data to determine the likelihood a queried peptide would be presented. Details on training are provided within Example 1 and 2 within the Exemplary embodiments.

In several embodiments, a peptide presentation module utilizes an RNN, which provides advantages for analyses of variable length sequences. Various architectures of RNN can be utilized. In various embodiments, an RNN architecture is one (or a combination) of: fully recurrent, long short-term memory (LSTM), gated recurrent unit (GRU), bidirectional LSTM and hierarchical recurrent network. By utilizing an RNN, peptides of variable length can be assessed within the same module architecture.

In many embodiments, a peptide (or set of peptides) is queried within the neural network to determine whether the sequence is predicted to be presented by a particular HLA allele, as determined by the data used to train the model. In some embodiments, the output layer contains a single neuron providing a likelihood of presentation between 0 and 1.

In a number of embodiments, a peptide presentation module is integrated with one or more modules: a binding affinity module, a cleavability module, and/or a gene expression module. The results of these modules can be combined with the analysis of sequence data to provide an overall presentation score. In some embodiments, a computational framework is a peptide presentation module integrated with a binding affinity module. In some embodiments, a computational framework is a peptide presentation module integrated with a cleavability module. In some embodiments, a computational framework is a peptide presentation module integrated with a gene expression module. In some embodiments, a computational framework is a peptide presentation module integrated with a binding affinity module and a cleavability module. In some embodiments, a computational framework is a peptide presentation module integrated with a binding affinity module and a gene expression module. In some embodiments, a computational framework is a peptide presentation module integrated with a cleavability module and a gene expression module. In some embodiments, a computational framework is a peptide presentation module integrated with a binding affinity module, a cleavability module, and a gene expression module.

### Binding Affinity Module

In several embodiments, a binding affinity module computes the likelihood of a peptide to bind to MHC I or MHC II, which is dependent on the HLA allele expressed. Accordingly, in some embodiments, a binding affinity module utilizes the peptide's sequence and is queried to determine if the sequence has affinity to bind to a particular HLA allele (or set of HLA alleles). To determine binding, in accordance with some embodiments, the module utilizes a recurrent neural network (RNN) that has been trained utilizing in vitro HLA allele/peptide binding affinity data. Training data may be obtained from HLA allele/peptide binding affinity experiments. In some embodiments, HLA allele/peptide binding affinity data utilized for training is derived from a database, such as the Immune Epitope Database (IEDB). Accordingly, in some embodiments, numerous HLA/peptide pairs are assessed for their binding affinity as measured by half maximal inhibitory concentration (IC₅₀) to train the binding affinity module. Details on training are provided within Example 1 and 2 within the Exemplary embodiments.

In several embodiments, a binding affinity module utilizes an RNN, which provides advantages for analyses of variable length sequences. Various architectures of RNN can be utilized. In various embodiments, an RNN architecture is one (or a combination) of: fully recurrent, long short-term memory (LSTM), gated recurrent unit (GRU), bidirectional LSTM and hierarchical recurrent network. By utilizing an RNN, peptides of variable length can be assessed within the same module architecture.

In many embodiments, a peptide (or set of peptides) is queried within the neural network to determine whether the sequence is predicted to have affinity for a particular HLA allele, as determined by the data used to train the model. In some embodiments, the output layer contains a single neuron providing a likelihood of affinity between 0 and 1. Results of the model can be utilized in isolation or integrated with a peptide presentation module and optionally other modules to provide a MHC presentation score.

### Cleavability Module

In several embodiments, a cleavability module to determines the likelihood that a cleavage reaction would occur to produce a peptide for MHC presentation. In some embodiments, cleavability is determined by the amino acid sequences that flank the peptide being assessed. Accordingly, in some embodiments, up to 24 flanking amino acids are considered, upstream the peptide and/or downstream the peptide. In various embodiments, 4, 5, 6, 7, 8, 9, 10, 11, or 12 amino acids upstream the peptide and/or 4, 5, 6, 7, 8, 9, 10, 11, or 12 amino acids downstream the peptide are considered.

In many embodiments, a neural network is built to determine cleavability. In some embodiments, a cleavability module determines the flanking amino acids from an appropriate source (e.g., a proteome database with protein sequences), encodes these determined amino acids, a processes them with hidden layers to output a probability score between 0 and 1. To train the model, in accordance with some embodiments, the ligandome of an antigen presenting cell line (e.g., a dendritic or a lymphoma cell line) is utilized to identify the most common flanking amino acids in each flanking position.

In a number of embodiments, a peptide (or set of peptides) is queried within the neural network to determine whether the flanking sequence of the peptide contains enrichment of amino acids are cleaved. In some embodiments, the output layer contains two neurons providing a likelihood of false (F) or true (T) cleavability between 0 and 1. Results of the model can be utilized in isolation or integrated with a peptide presentation module and optionally other modules to provide a MHC presentation score.

### Gene Expression Module

In several embodiments, an expression module determines the relative expression level of a peptide. It is now known that expression levels correlate with MHC peptide presentation. When a gene is highly expressed, peptides of the gene product are more likely to be presented than genes expressed at lower rates. Accordingly, in some embodiments, the expression level of a gene from which the peptide is derived is determined, which is utilized to determine the likelihood of presentation.

It was further discovered that some peptides derived from lowly expressed genes are also highly presented. These peptides are often products of extracellular proteins and blood protein constituents. Accordingly, in some embodiments, gene expression of extracellular proteins and blood protein constituents are “corrected” to account for their bias of being highly presented despite their gene being lowly expressed. Any appropriate mechanism to account for this bias can be utilized. In some embodiments, expression of extracellular proteins and blood proteins constituents are artificially set to high expression levels. For example, in various embodiments, gene expression values of genes under one or more of the following GO terms are set to 50 transcripts per million (TPM): extracellular space (0005615), blood microparticle (0072562), secretory granule lumen (0034774), cytoplasmic vesicle lumen (0060205), and/or extracellular matrix (0031012).

Expression of genes can be determined by various methods. In some embodiments, gene expression is determined empirically. Accordingly, the gene expression of the peptide being assessed is determined from personalized RNA sequencing data. In some embodiments, gene expression is inferred. Accordingly, the gene expression of the peptide being assessed is determined from external RNA sequencing data. It was found that gene expression matched tissue, and in some cases, gene expression from unmatched tissue, can provide a robust indication of MHC presentation. When gene expression is inferred, in some embodiments, a gene expression “dictionary” having stored relative expression values of genes is utilized to determine relative expression of a peptide. In some embodiments, a logistic regression model is trained to differentiate highly expressed peptides from decoys.

In many embodiments, a peptide (or set of peptides) is queried within a logistic regression incorporating a gene expression dictionary to estimate the relative peptide expression values. In some embodiments, the output layer contains a single neuron providing an expression value (e.g., TPM). Results of the model can be utilized in isolation or integrated with a peptide presentation module and optionally other modules to provide a MHC presentation score.

### Module Integration

In several embodiments, a peptide presentation module and one or more modules are integrated within a computational framework to produce a MHC I or MHC II presentation score. As explained within the Exemplary embodiments, integrating a binding infinity module and/or a cleavability module and/or an expression module with a peptide presentation module can result in better MHC presentation prediction. To integrate modules, in some embodiments, input data, including peptide sequence, HLA allele, and/or peptide gene data, is shared amongst the modules. In addition, in some embodiments, an integrated framework includes an RNN layer to encode each ligand peptide sequence. In some embodiments, the output of each module is concatenated to merge their information to yield an ultimate output layer indicating likelihood of presentation.

Process 100 determines (105) a MHC I or MHC II presentation score for each peptide sequence queried within the computational framework. Accordingly, in various embodiments, presentation scores are determined from one or more computational modules utilized. In some embodiments, the output presentation score is between 0 and 1 indicating how likely a query peptide is to be presented by a specific HLA allele. To increase human interpretability and enable comparison across different peptide lengths, in some embodiments, the framework's output can be represented as a percentile score. In some embodiments, a percentile score is generated by comparing the raw output score to a score distribution generated from length-matched random human peptides. The higher the percentile, the more likely the peptide will be presented by a cell HLA-DR complex. Based on the presentation score, in accordance with some embodiments, peptides are compared and/or ranked.

Process 100 optionally performs downstream applications on one more peptides on the basis of their presentation score. In some embodiments, based on its presentation score, a peptide is synthesized for further analysis. In some embodiments, based on its presentation score, a peptide sequence is utilized to develop cancer vaccines. In some embodiments, based on its presentation score, a peptide sequence is utilized to develop vaccines against infectious pathogens. In some embodiments, based on its presentation score, a peptide sequence is used to develop T cell therapeutics. In some embodiments, based on its presentation score, a peptide sequence is utilized to develop a treatment to induce tolerance to autoantigen. In some embodiments, based on its presentation score, a peptide sequence is utilized to develop a treatment to induce tolerance to exogenous antigen that is involved in autoimmunity and/or allergies.

While specific examples of processes for determining a MHC presentation score are described above, one of ordinary skill in the art can appreciate that various steps of the process can be performed in different orders and that certain steps may be optional according to some embodiments of the invention. As such, it should be clear that the various steps of the process could be used as appropriate to the requirements of specific applications. Furthermore, any of a variety of processes for determining a MHC presentation score appropriate to the requirements of a given application can be utilized in accordance with various embodiments of the invention.

### Applications

Various embodiments are directed towards utilizing MHC I or MHC II peptide presentation scores in various applications. In some embodiments, MHC presentation scores can identify immunogenic peptides, which can be useful in vaccine development and/or T cell therapies. In some embodiments, MHC presentation scores can identify peptides that could cause autoimmunity and/or allergies and thus treatments can be designed to induce tolerance to those peptides.

### Peptide Synthesis

Peptides can be synthesized chemically by a number of methods. One common method is to use solid-phase peptide synthesis (SPPS). In many embodiments, a peptide is synthesized first as a linear peptide utilizing solid-phase peptide synthesis (SPPS). Any appropriate SPPS protocol can be utilized. The solid support can be any appropriate solid support, such as (for example) the Merrifield resin, the PAM resin, the Wang resin, or 2-chlorotrityl resin. Any appropriate protecting groups can be utilized, such as (for example) Fmoc or Boc.

Peptides can also be synthesized utilizing molecular tools and a host cell. Nucleic acid sequences corresponding with antigenic peptides can be synthesized. In some embodiments, nucleic acids synthesized in in vitro synthesizers (e.g., phosphoramidite synthesizer), bacterial recombination system, or other suitable methods. Furthermore, synthesized nucleic acids can be purified and lyophilized, or kept stored in a biological system (e.g., bacteria, yeast). For use in a biological system, synthetic nucleic acid molecules can be inserted into a plasmid vector, or similar. A plasmid vector can also be an expression vector, wherein a suitable promoter and a suitable 3′-polyA tail is combined with the transcript sequence.

Embodiments are also directed to expression vectors and expression systems that produce antigenic peptides or proteins. These expression systems can incorporate an expression vector to express transcripts and peptides in a suitable expression system. Typical expression systems include bacterial (e.g., E. coli), insect (e.g., SF9), yeast (e.g., S. cerevisiae), animal (e.g., CHO), or human (e.g., HEK 293) cell lines. RNA and/or peptides can be purified from these systems using standard biotechnology production procedures.

### Vaccine Development and Administration

A number of embodiments utilize methods to develop and administer vaccines against antigenic peptides. In various embodiments, infectious pathogen antigen and/or cancer neoantigen peptides are screened by their MHC presentation score then utilized to develop a vaccine against the peptide. Accordingly, various embodiments contemplate administering immunogenic compositions to individuals, proposed to be suitable for use as a vaccine, prepared using one or more antigenic peptides that comprise peptide sequences selected on the basis of their MHC presentation score. In some embodiments, antigenic peptides would further include flanking sequences that confer high cleavability. In some embodiments, antigenic peptides can be used in combination with other secreted virulence proteins, surface proteins or immunogenic fragments thereof. In certain aspects, antigenic material is extensively dialyzed to remove undesired small molecular weight molecules and/or lyophilized for more ready formulation into a desired vehicle.

The preparation of vaccines that contain polypeptide or peptide sequence(s) as active ingredients is generally well understood in the art, as exemplified by U.S. Pat. Nos. 4,608,251; 4,601,903; 4,599,231; 4,599,230; and 4,596,792; each of which is incorporated herein by reference. Typically, such vaccines are prepared as injectables either as liquid solutions or suspensions: solid forms suitable for solution in or suspension in liquid prior to injection may also be prepared. The preparation may also be emulsified. The active immunogenic ingredient is often mixed with excipients that are pharmaceutically acceptable and compatible with the active ingredient. Suitable excipients are, for example, water, saline, dextrose, glycerol, ethanol, or the like and combinations thereof. In addition, if desired, the vaccine may contain amounts of auxiliary substances such as wetting or emulsifying agents, pH buffering agents, or adjuvants that enhance the effectiveness of the vaccines. In specific embodiments, vaccines are formulated with a combination of substances, as described in U.S. Pat. Nos. 6,793,923 and 6,733,754, each of which is incorporated herein by reference.

Vaccines may be conventionally administered parenterally, by injection, for example, either subcutaneously or intramuscularly. Additional formulations which are suitable for other modes of administration include suppositories and, in some cases, oral formulations. For suppositories, traditional binders and carriers may include, for example, polyalkalene glycols or triglycerides: such suppositories may be formed from mixtures containing the active ingredient in the range of about 0.5% to about 10%, preferably about 1% to about 2%. Oral formulations include such normally employed excipients as, for example, pharmaceutical grades of mannitol, lactose, starch, magnesium stearate, sodium saccharine, cellulose, magnesium carbonate and the like. These compositions take the form of solutions, suspensions, tablets, pills, capsules, sustained release formulations or powders and contain about 10% to about 95% of active ingredient, preferably about 25% to about 70%.

Vaccine compositions would normally be administered as pharmaceutically acceptable compositions that include physiologically acceptable carriers, buffers or other excipients. As used herein, the term “pharmaceutically acceptable” refers to those compounds, materials, compositions, and/or dosage forms which are, within the scope of sound medical judgment, suitable for contact with the tissues of human beings and animals without excessive toxicity, irritation, allergic response, or other problem complications commensurate with a reasonable benefit/risk ratio. The term “pharmaceutically acceptable carrier,” means a pharmaceutically acceptable material, composition or vehicle, such as a liquid or solid filler, diluent, excipient, solvent or encapsulating material, involved in carrying or transporting a chemical agent.

An effective amount of therapeutic or prophylactic composition is determined based on the intended goal. The term “unit dose” or “dosage” refers to physically discrete units suitable for use in a subject, each unit containing a predetermined quantity of the composition calculated to produce the desired responses discussed above in association with its administration, i.e., the appropriate route and regimen. The quantity to be administered, both according to number of treatments and unit dose, depends on the protection desired.

Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition.

Typically, vaccines are administered in a manner compatible with the dosage formulation, and in such amount as will be therapeutically effective and immunogenic. The quantity to be administered depends on the subject to be treated, including the capacity of the individual's immune system to induce a T cell response and the degree of protection desired. Precise amounts of active ingredient required to be administered depend on the judgment of the practitioner. However, suitable dosage ranges are of the order of several hundred micrograms of active ingredient per vaccination. Suitable regimes for initial administration and booster shots are also variable, but are typified by an initial administration followed by subsequent inoculations or other administrations.

Upon formulation, solutions will be administered in a manner compatible with the dosage formulation and in such amount as is therapeutically or prophylactically effective. The formulations are easily administered in a variety of dosage forms, such as the type of injectable solutions described within.

The manner of application may be varied widely. Any of the conventional methods for administration of a vaccine are applicable. These are believed to include oral application within a solid physiologically acceptable base or in a physiologically acceptable dispersion, parenterally, by injection and the like. The dosage of the vaccine will depend on the route of administration and will vary according to the size and health of the subject.

In certain instances, it will be desirable to have multiple administrations of the vaccine, e.g., 2, 3, 4, 5, 6 or more administrations. The vaccinations can be at 1, 2, 3, 4, 5, 6, 7, 8, to 5, 6, 7, 8, 9, 10, 11, 12 twelve week intervals, including all ranges there between. Periodic boosters at intervals of 1-5 years will be desirable to maintain protective levels of the antibodies. The course of the immunization may be followed by assays for antibodies against the antigens, as described in U.S. Pat. Nos. 3,791,932; 4,174,384 and 3,949,064, each of which is incorporated herein by reference.

A given composition may vary in its immunogenicity. It is often necessary therefore to boost the host immune system, as may be achieved by coupling a peptide or polypeptide to a carrier. Exemplary and preferred carriers are keyhole limpet hemocyanin (KLH) and bovine serum albumin (BSA). Other albumins such as ovalbumin, mouse serum albumin, or rabbit serum albumin can also be used as carriers. Means for conjugating a polypeptide to a carrier protein are well known in the art and include glutaraldehyde, m-maleimidobenzoyl-N-hydroxysuccinimide ester, carbodiimide, and bis-diazotized benzidine.

The immunogenicity of polypeptide or peptide compositions can be enhanced by the use of non-specific stimulators of the immune response, known as adjuvants. Suitable adjuvants include all acceptable immunostimulatory compounds, such as cytokines, toxins, or synthetic compositions. A number of adjuvants can be used to enhance an antibody response against antigenic peptides. Adjuvants can (1) trap the antigen in the body to cause a slow release; (2) attract cells involved in the immune response to the site of administration; (3) induce proliferation or activation of immune system cells; or (4) improve the spread of the antigen throughout the subject's body.

Examples of adjuvants include, but are not limited to, complete Freund's adjuvant (a non-specific stimulator of the immune response containing killed Mycobacterium tuberculosis), incomplete Freund's adjuvants, aluminum hydroxide, oil-in-water emulsions, water-in-oil emulsions, mineral salts, polynucleotides, and natural substances. Others adjuvants or methods are exemplified in U.S. Pat. Nos. 6,814,971, 5,084,269, 6,656,462, each of which is incorporated herein by reference.

In some embodiments, vaccines are directed towards dendritic cell therapy, which provokes anti-tumor responses by causing dendritic cells to present tumor antigens to lymphocytes, which activates them, priming them to kill other cells that present the antigen. Dendritic cells are antigen presenting cells (APCs) in the mammalian immune system. In cancer treatment they aid cancer antigen targeting.

One method of inducing dendritic cells to present tumor antigens is by vaccination with MHC II presenting peptides. Peptides can be administered as described herein, especially in combination with adjuvants (highly immunogenic substances) to increase the immune and anti-tumor responses. Other adjuvants include proteins or other chemicals that attract and/or activate dendritic cells, such as granulocyte macrophage colony-stimulating factor (GM-CSF).

### Induction of Tolerance

A number of embodiments are directed towards identify MHC presenting peptides involved with autoimmune and/or allergic responses and then using those peptides to induce tolerance (i.e., desensitize response) to the peptides. It is known that a number of HLA alleles (especially MHC II HLA alleles) are involved in autoimmune and allergic responses. Autoantigens in type I diabetes, multiple sclerosis, rheumatoid arthritis, and systemic lupus erythematosus have been identified to correlate with various MHC II HLA alleles. Likewise, celiac disease, gluten sensitivity, dust mite allergies, pet allergies, and peanut allergies have been identified to correlate with various MHC II HLA alleles. Accordingly, peptides associated with these autoantigens and allergens can be screened to identify peptides responsible for these responses.

In some embodiments, individual are treated for an autoimmune disease or allergy by administering to an individual MHC II presented peptide antigens in way to sensitive the individual to the antigens. Intradermic, subdermal, and/or intramuscular administrations of peptides in ascending doses can be utilized to induce tolerance to certain autoimmune diseases and allergies.

### T Cell Therapy

Several embodiments are directed towards identifying MHC presenting peptides that can be utilized in a T cell therapy, which may be utilized in the treatments of various cancers. T cell receptors (TCR) recognize MHC I and MHC II complexes with HLA alleles and presented peptide. When an antigenic peptide is presented, the TCR induces an immune response against the antigenic peptide. Accordingly, presented peptides, as identified in embodiments described herein, can be utilized in T cell therapies.

In some embodiments, T cell can be screened to identify T cells with a T cell receptor (TCR) that recognizes a MHC with a particular presented peptide, such as peptide that would be presented as neoantigen on a cancer cell. Once T cells capable of recognizing peptides are identified, they can be isolated and propagated for a T cell therapy. In some embodiments, a patient's own T cells are removed from the patient and then screened and then propagated.

In some embodiments, a T cell can be engineered to express a TCR-like receptor to recognize a MHC with a particular presented peptide, such as peptide that would be presented as neoantigen on a cancer cell. Engineered T cells that recognize particular presented peptides in the MHC are typically referred to as TCR-like chimeric antigen receptor (CAR) T-cells, as they include a chimeric receptor similar to a TCR that is specifically designed to identify a particular presented peptide. Engineered T cells can be propagated for T cell therapies. In some embodiments, a patient's own T cells are removed from the patient and then engineered and then propagated.

In some embodiments, propagated T cells capable of recognizing MHC complex with a particular presented neoantigen are utilized to treat a patient having cancer. Accordingly, T cells are administered to the patient intravenously and/or intratumorally. The T cells would recognize the MHC complex with a particular presented neoantigen on the cancer cells, inducing the patient's immune system to respond to and attack the cancer.

### EXEMPLARY EMBODIMENTS

The embodiments of the invention will be better understood with the several examples provided within. Many exemplary results of processes that identify HLA antigen peptides are described. Validation results are also provided.

### Example 1: Predicting HLA Class II Antigen Presentation Through Integrated Deep Learning

Major histocompatibility complex class II (MHC-II) is a glycoprotein complex on the surface of professional antigen-presenting cells that displays short antigen peptides to CD4⁺ helper T cells. Human antigen-presenting cells, such as dendritic cells and B cells, rely in large part on HLA class II (HLA-II) for the presentation of antigens to CD4⁺ T cells. This human form of MHC-II can also be conditionally expressed by many other human cell types, including tumor cells. Antigen presentation by these HLA-II molecules on human cells involves three loci on chromosome 6 (DR, DQ and DP) which encode the corresponding heterodimeric proteins through combinations of alpha and beta chains.

Such HLA-II presentation of endogenous and exogenous antigenic peptides is essential for robust immune responses against diverse pathogens, and is also of major significance for autoimmunity and antitumor immunity. For example, recent mass spectrometry (MS)-based studies have shown that lymphoma and melanoma cells present somatically mutated cancer peptides (neoantigens) in the context of HLA-II. CD4⁺ T cell recognition of neoantigens is commonly observed across diverse human tumor types and in animal models, which underscores the potential clinical relevance of HLA-II-restricted neoantigens for cancer immunotherapy. Furthermore, neoantigens presented by HLA-II elicit potent antitumor responses in T cells from immunized patients. Reliably identifying presentation by HLA-II would allow prioritization of vaccine candidates and potentially identify likely responders to immune therapies.

Owing to the high cost and technical challenge of experimentally testing all possible peptide candidates, researchers have attempted to computationally identify HLA-II peptides with machine-learning algorithms. However, nearly all current HLA-II prediction methods rely on in vitro binding affinities of recombinant HLA-II molecules as surrogates, and therefore ignore other contributing factors including gene expression and protease cleavage preferences. When combined with the remarkably variable length of HLA-II peptides and their binding promiscuity, this deficiency makes HLA-II antigen presentation prediction task especially challenging. For example, the latest benchmarks report average receiver operating characteristic area under the curve (ROC-AUC or AUC) of ˜0.83 for current prevailing HLA-II prediction models, even when validated on in vitro binding data.

In this study, a deep neural network trained to accurately predict the likelihood of a peptide being presented by HLA-II complexes is described, which is referred to as MARIA throughout. Rather than relying on in vitro binding affinities alone, MARIA is trained on naturally presented HLA-II peptides (ligands) identified from human samples profiled by liquid chromatography-tandem mass spectrometry (LC-MS/MS). Despite some inherent limitations of MS methods, peptide ligand sequences identified by antigen presentation profiling currently provide the closest sample population to the true presented ligands. Such training data could enable new prediction models to consider multiple relevant features including expression and binding affinities. Here it is shown that MARIA allows robust and more accurate HLA-II prediction, and that its performance gains are achieved by combining these improved training data with a new supervised machine learning model using a multimodal recurrent neural network (RNN).

### Performance of Binding-Based HLA-II Peptide Prediction Methods

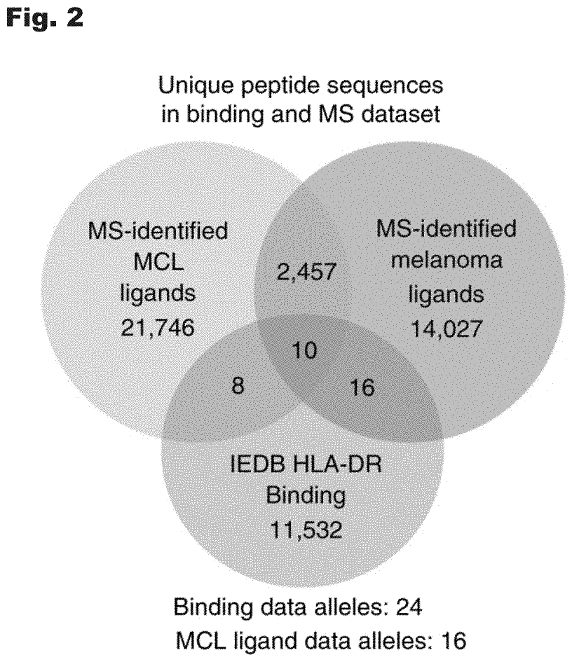

Immunoprecipitation of MHC molecules followed by peptide elution and LC-MS/MS analysis enables direct recovery of peptides presented by HLA-II in primary cells. In comparison to traditional in vitro binding affinity assays, MS-based profiling methods can rapidly yield large datasets of peptides actively presented by cells or tissues. The Immune Epitope Database (IEDB), the largest public depository of results of HLA binding assays, contains quantitative HLA-DR binding affinities for ˜12,000 non-redundant peptide sequences (as of December 2018) (see R. Vita Nucleic Acids Res. 43, D405-D412 (2015), the disclosure of which is incorporated herein by reference). By comparison, two studies employing HLA-DR immunoprecipitation and MS analysis identified >23,000 and >16,000 non-redundant peptide sequences, respectively (FIG. 2).

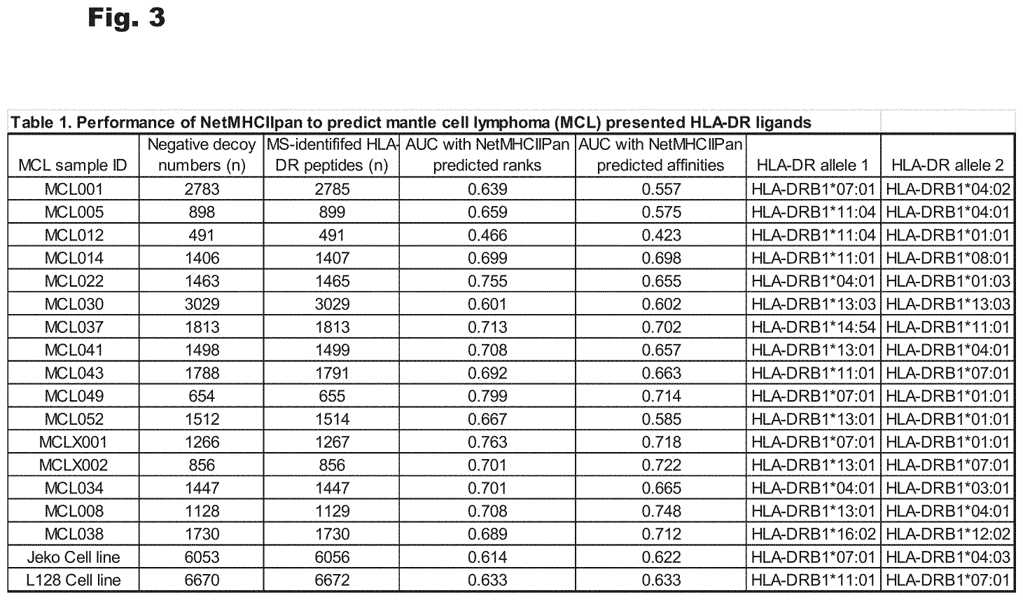

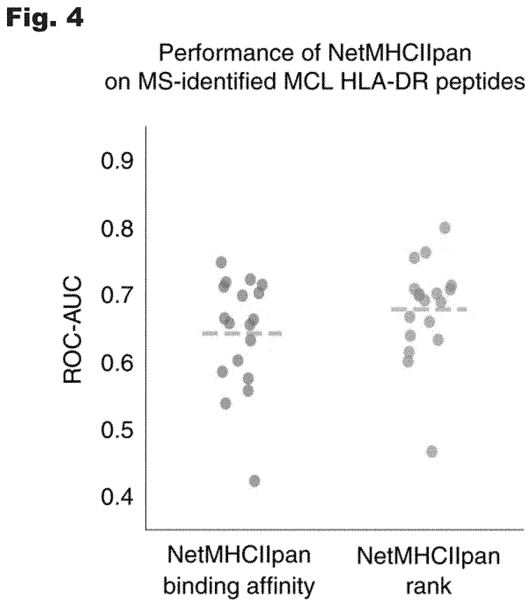

The performance of the HLA binding affinity prediction tool was tested. The tool was trained on in vitro binding data to identify HLA-DR ligands presented by human antigen-presenting cells. NetMHCIIpan (R. Marty, et al., Cell 175, 416-428 (2018), the disclosure of which is incorporated herein by reference), a widely used HLA-II binding prediction method, was applied to predict the binding affinity of HLA-DR ligands experimentally identified from 18 mantle cell lymphomas (MCLs) representing 16 HLA-DR alleles (Table 1 in FIG. 3). The AUC of NetMHCIIpan was assessed using MS-identified ligands as true positives and randomly selected length-matched human peptide sequences (decoys) as negative examples. For each HLA allele and peptide sequence pair, NetMHCIIpan generates a binding affinity and binding ranks. Separately using these two values, the average AUCs were 0.64 and 0.68 for NetMHCIIpan binding affinities and ranks, respectively (FIG. 4). Binding ranks showed better prediction performance for ligand presentation (P=0.003), but nevertheless had mediocre accuracy in predicting true HLA-II ligands.

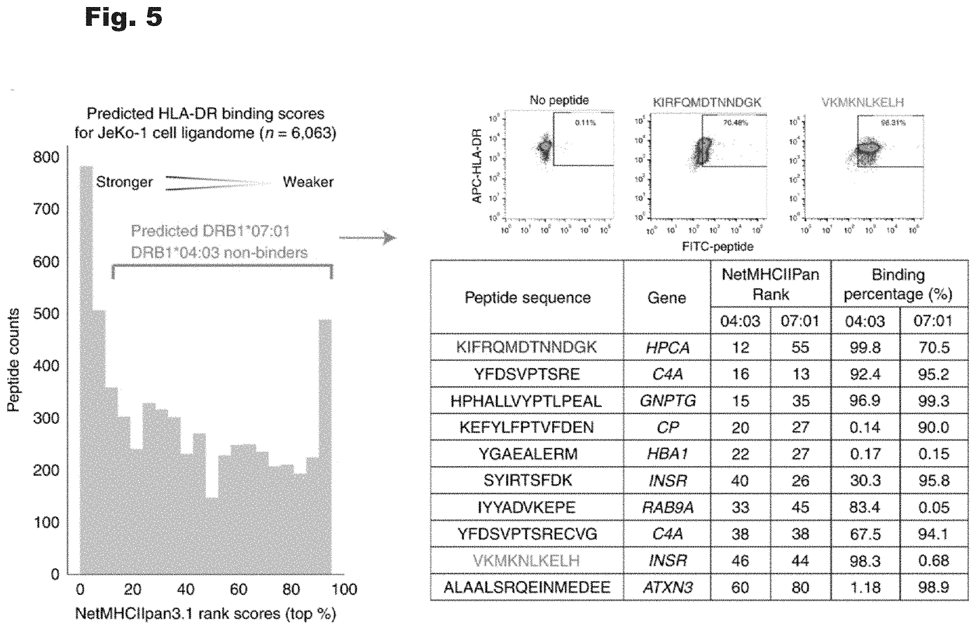

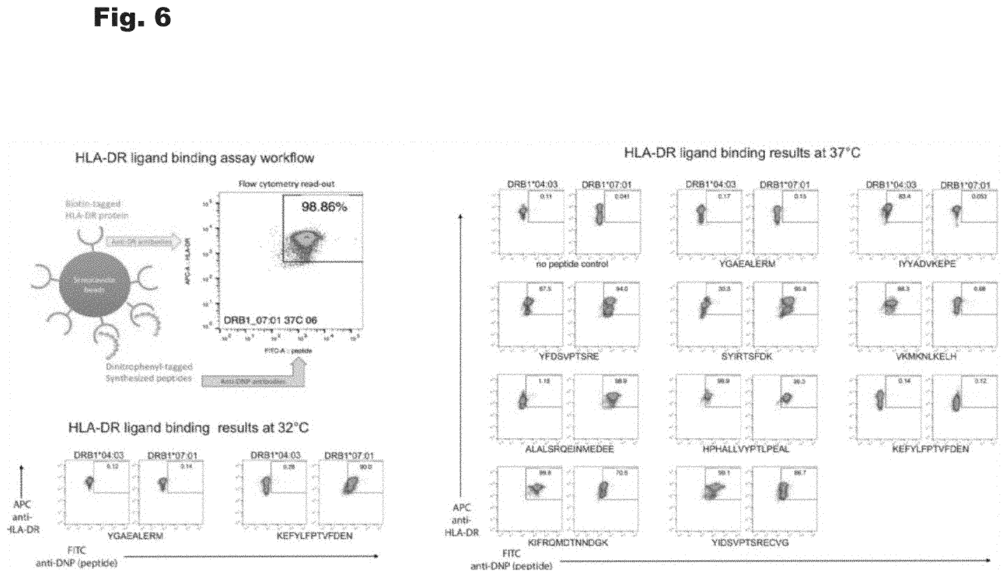

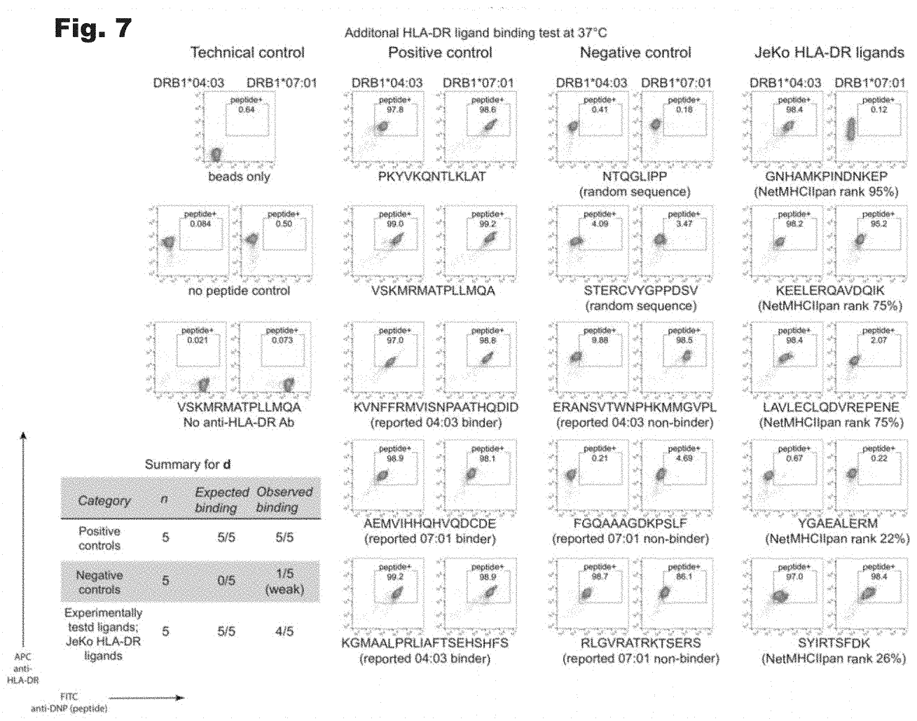

The performance of NetMHCIIpan was further tested on >6,000 HLA-DR ligands discovered through deep profiling of the MCL-derived JeKo-1 cell line. When using the recommended threshold of binding affinities (top 10% ranks), NetMHCIIpan labeled only ˜22% of the ligands as positive (FIG. 5). To confirm that the MS-identified peptides were indeed true ligands despite their weak NetMHCIIpan-predicted binding affinities, the binding of a subset of peptides with weak NetMHCIIpan scores along with positive and negative controls were experimentally validated (FIGS. 6 and 7). Remarkably, nine of ten synthesized peptides strongly bound to one or more cognate HLA-DR alleles in vitro, confirming the fidelity of the underlying MS data (FIGS. 5 and 7). Thus, NetMHCIIpan has limited accuracy for predicting antigen presentation when applied to MS-based datasets.

### Development of MARIA

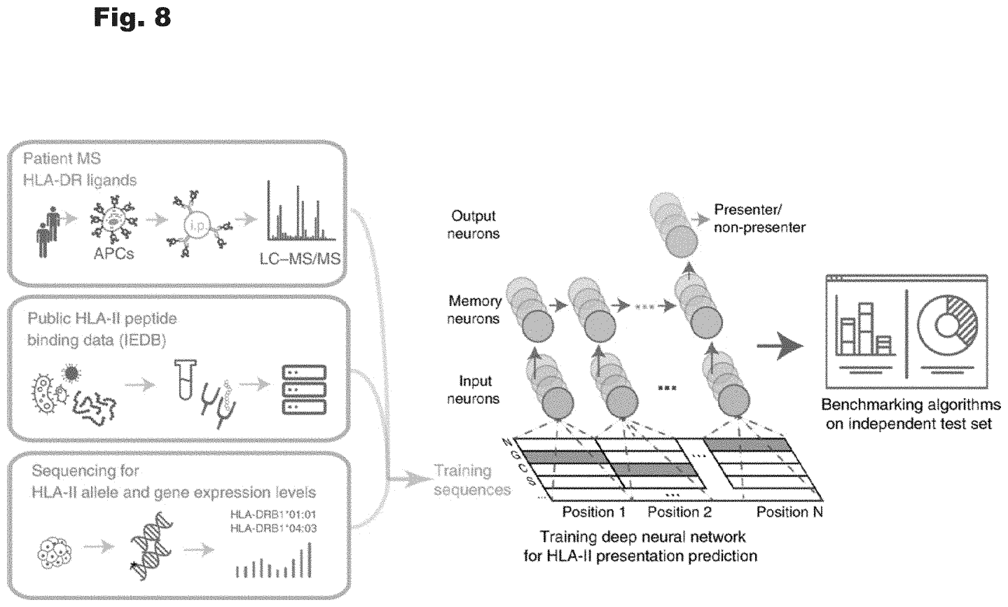

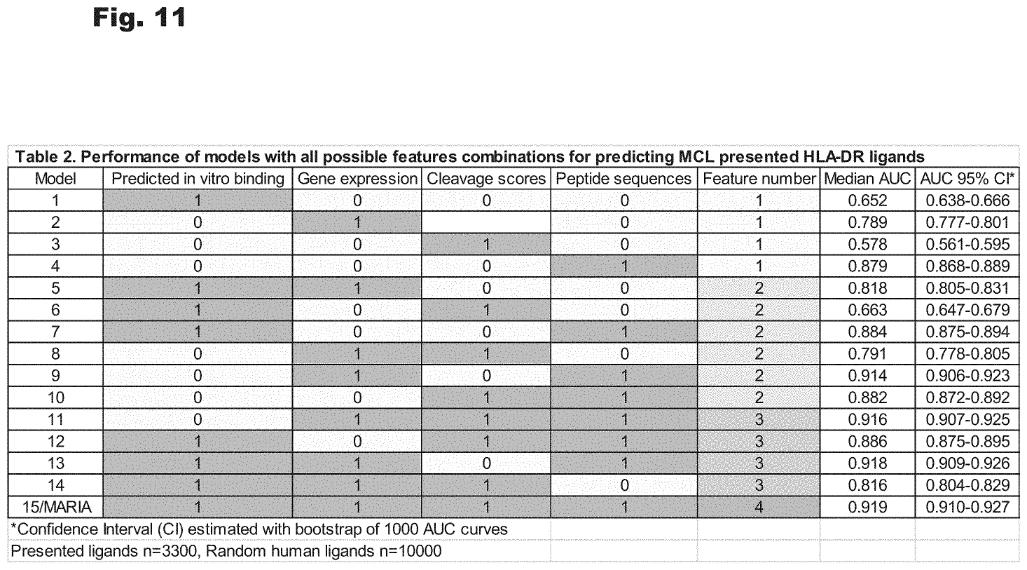

To improve upon previous HLA-II prediction methods MARIA was developed to predicting utilizing active HLA-II presentation in vivo, rather than from in vitro binding affinities. It was therefore examined whether HLA-II prediction could be refined by learning directly from MS-based antigen presentation profiling datasets, in addition to traditional HLA binding affinity data. Additionally, it was tested whether gene expression and protease cleavage signatures also have utility in predicting HLA-II peptide presentation. MARIA was trained using the HLA-II ligands identified by MS-based antigen presentation profiling, along with empirical in vitro HLA binding measurements, and gene expression levels (FIGS. 8, 9, 10A and 10B). Given the challenges associated with the high variability in the length of HLA-II peptide ligands (8-26 amino acids), a recurrent neural network (RNN) framework was used. RNN is a form of deep learning that excels at handling variable-length sequence data (FIG. 8). To prevent model overfitting owing to similarities in the training and validation sequences, any peptides in the cross-validation set that were a substring or highly similar to a training peptide were filtered out. The performance of the full model was evaluated, as well as other models trained on each possible combination of biological features (Table 2 in FIG. 11).

### Impact of Gene Expression Levels

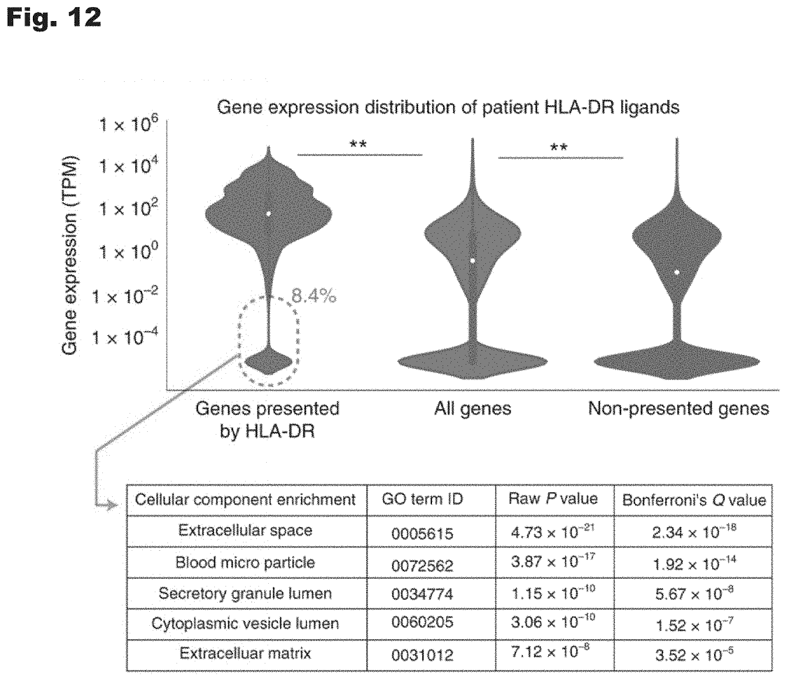

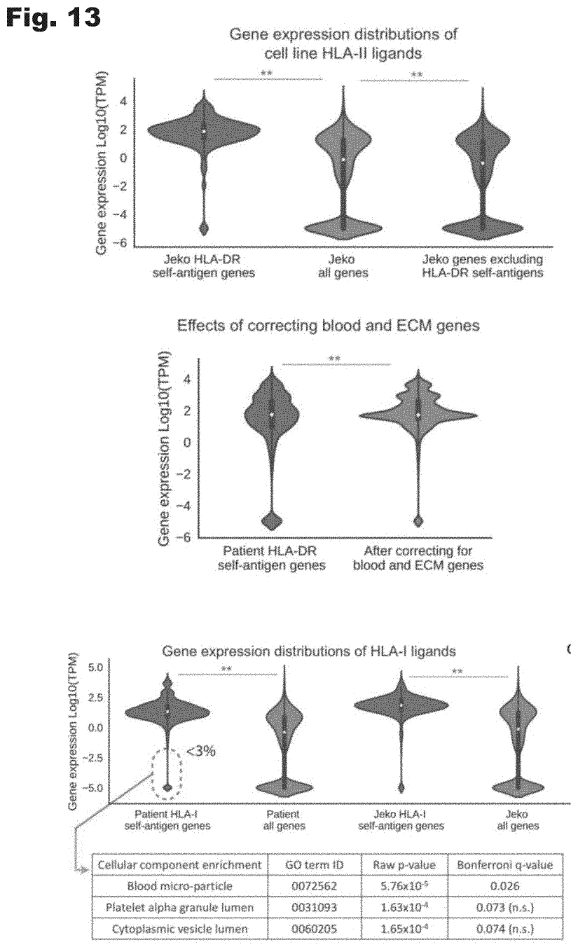

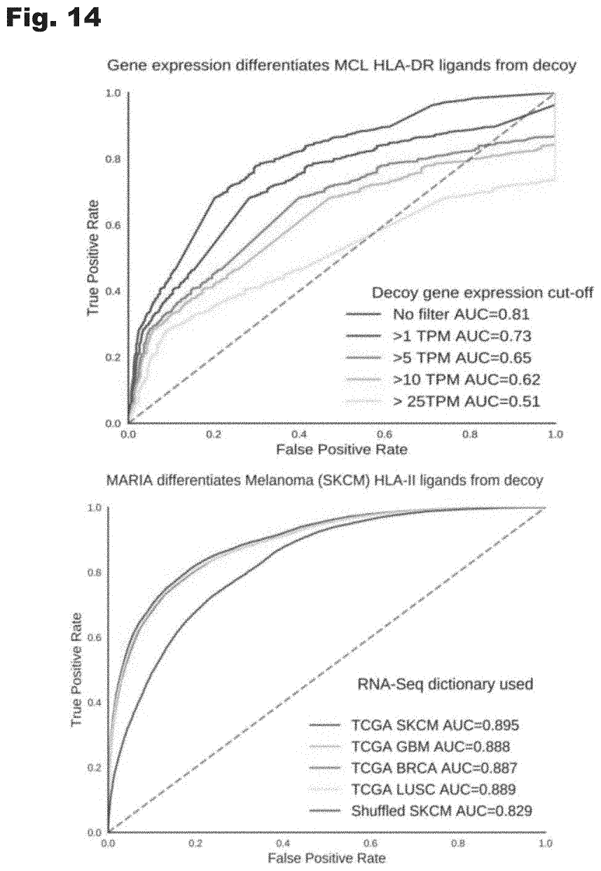

It was observed that gene expression levels of recovered HLA-DR ligands were significantly higher than both non-presented genes and random genes (FIGS. 12 and 13, 14 and 15). Nevertheless, 8.4% of peptide ligands were encoded by genes with extremely low RNA expression levels in tumor cells (<0.1 transcripts per million (TPM)). Consistent with the known role of HLA-II molecules in sampling and presenting extracellular antigens, the presented ligands from these outliers were highly enriched for extracellular proteins and blood microparticles (P<2×10⁻¹⁴, FDR-corrected hypergeometrical test; FIGS. 12 and 13). Gene expression levels was therefore included in the model and applied a correction to address presentation of extracellular proteins or blood particles (FIG. 13). Gene expression levels alone achieved an AUC of 0.81 when differentiating presented ligands from random human peptides (as detailed below). Not surprisingly, gene expression values had much weaker discriminatory power after removing lowly expressed genes in negative decoys (FIG. 14).

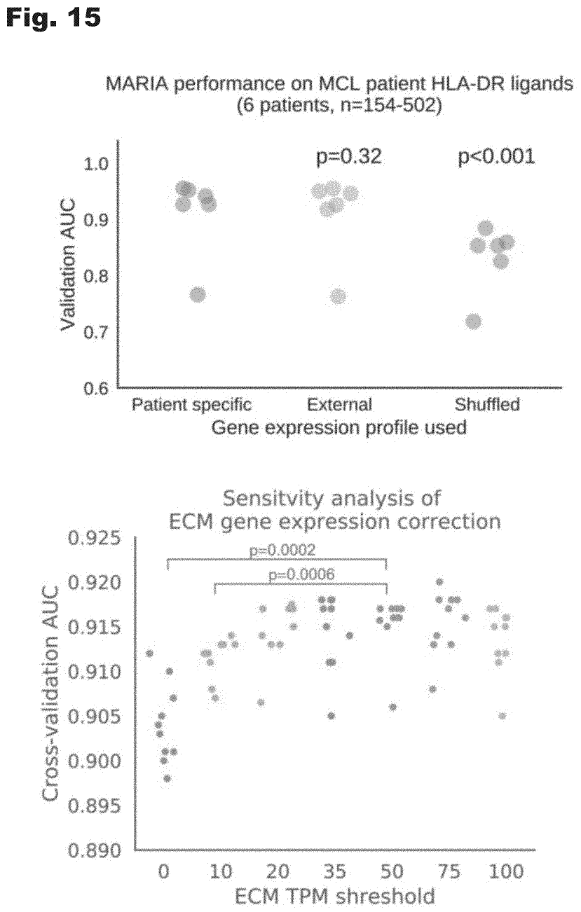

RNA sequencing (RNA-seq) gene expression profiles for six patients with MCL was analyzed. MARIA AUCs did not differ significantly between using patient-specific RNA-seq and an external RNA-seq profile (FIG. 15). Furthermore, only a modest degradation in prediction performance was observed when using tissue-mismatched gene expression values from a generalized reference database as compared to tissue-matched gene expression data (FIG. 14; change of AUC <1%, not significant). Supplementary Note 1 contains a detailed discussion for the predictive power of gene expression for HLA-II presentation.

### Impact of Cleavage Signatures

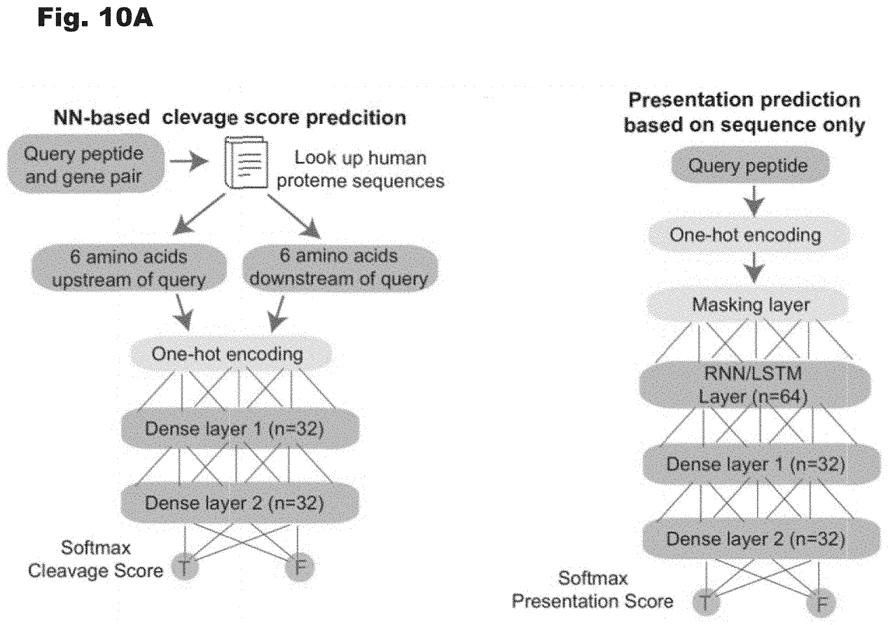

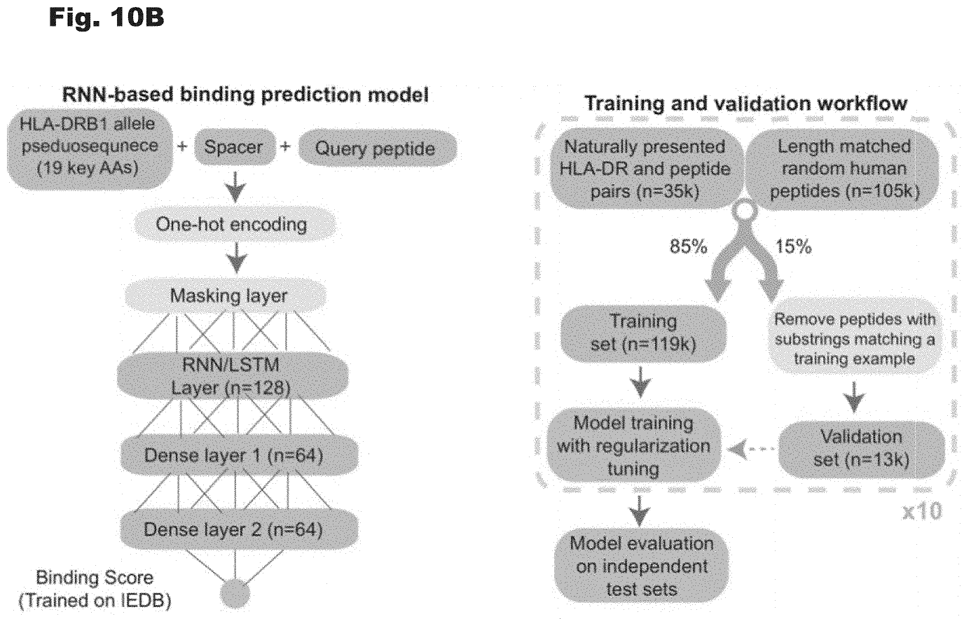

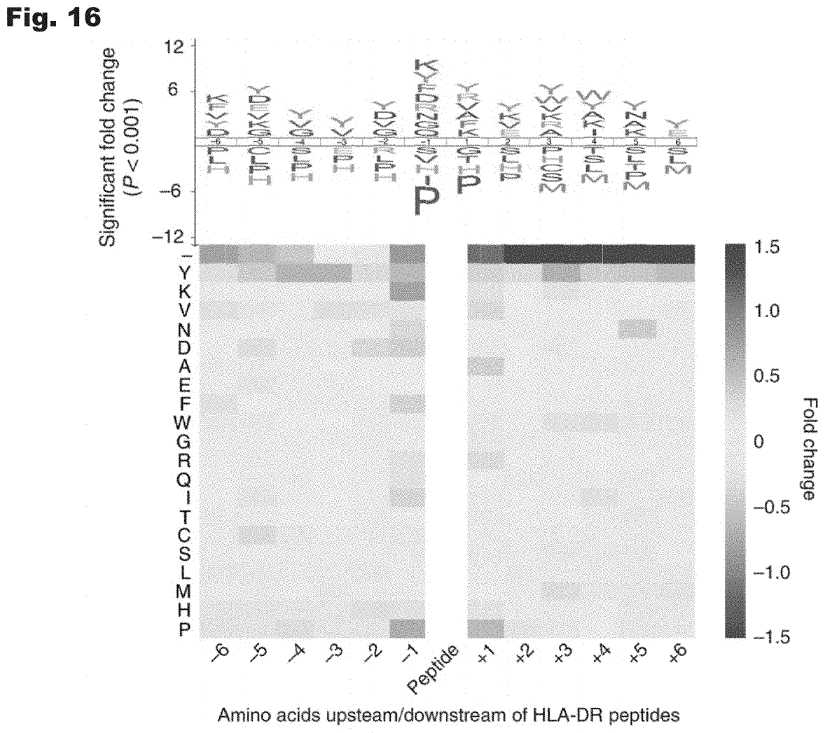

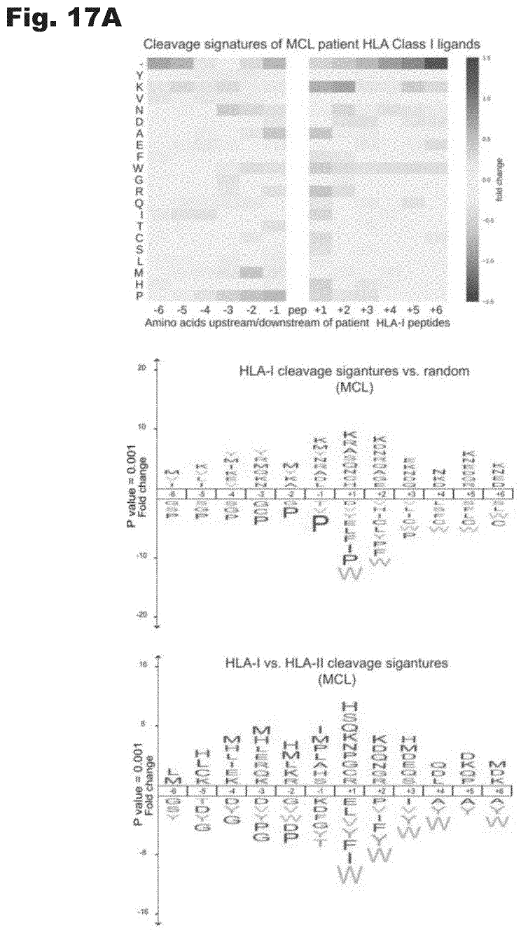

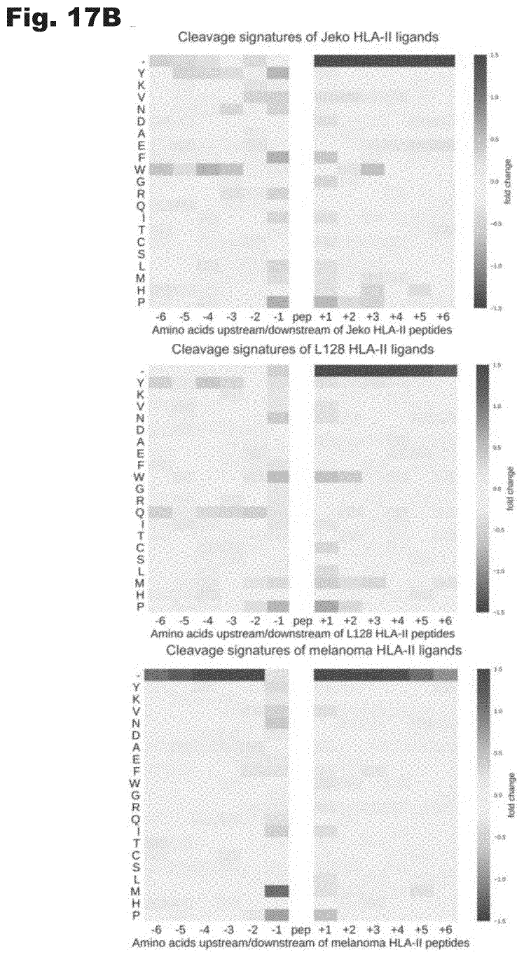

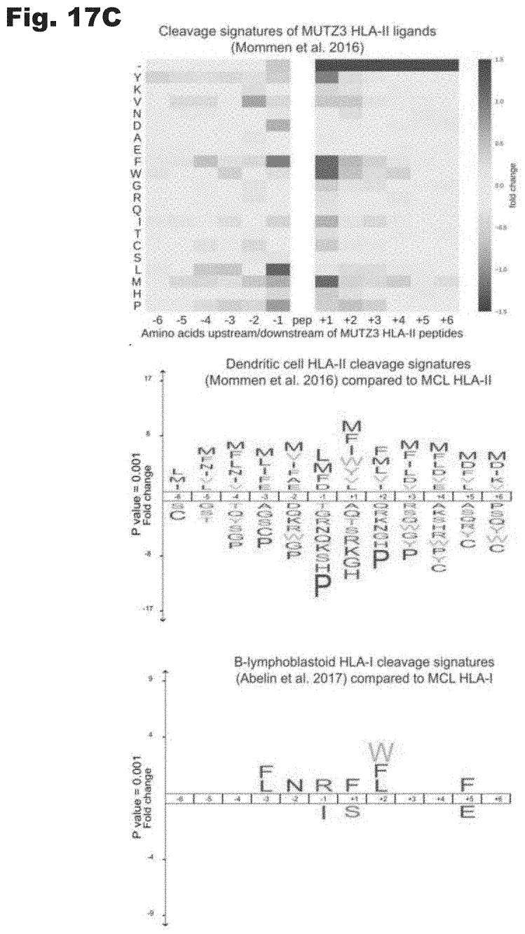

It was next assessed whether information from the flanking residues of a peptide could further improve predictive performance. Both significant enrichment and depletion of certain amino acids at specific residues immediately upstream of the ligand N terminus or downstream of its C terminus was observed (FIG. 16). For example, tyrosines were significantly enriched in sequences immediately flanking both termini of presented ligands (P<0.001), whereas histidines and prolines were generally absent from these regions (P<0.001). Among peptides presented by HLA-II, significant enrichment of those derived from the C termini of the mature proteins was observed (indicated as ‘-’ at +1 to +6 position; average fold change >150%; P<1×10⁻⁵). As these flanking sequences are not directly involved in HLA complex binding of peptide ligands, the observed enrichments likely reflect the cleavage preferences of proteases involved in processing proteins for presentation. Of note, these cleavage signatures were distinct for HLA-I and HLA-II ligands (FIG. 17), consistent with their distinct cleavage and processing pathways. Therefore, to capture the added predictive information from flanking residues, a dedicated neural network for assigning HLA-DR cleavage scores from a given peptide sequence was developed (FIGS. 10A, 17B, and 17C).

### MARIA Data Integration Framework

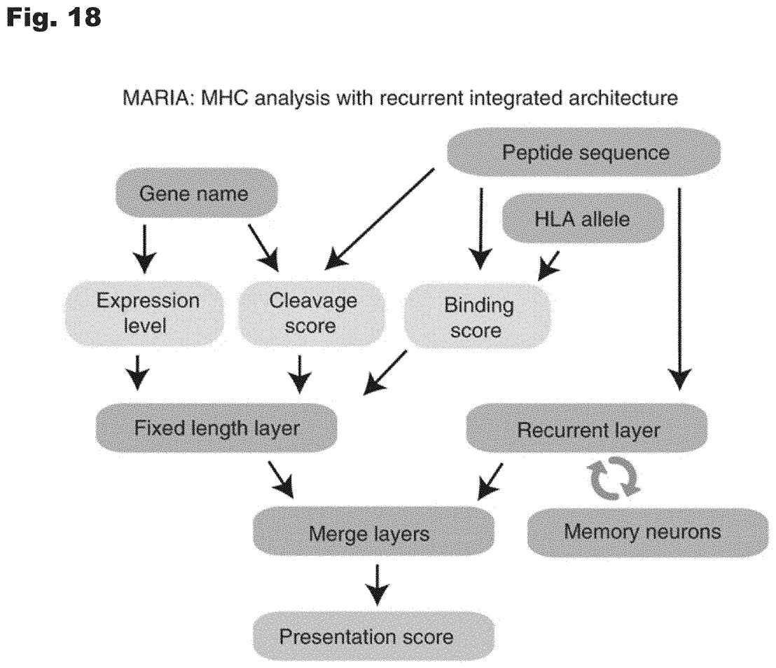

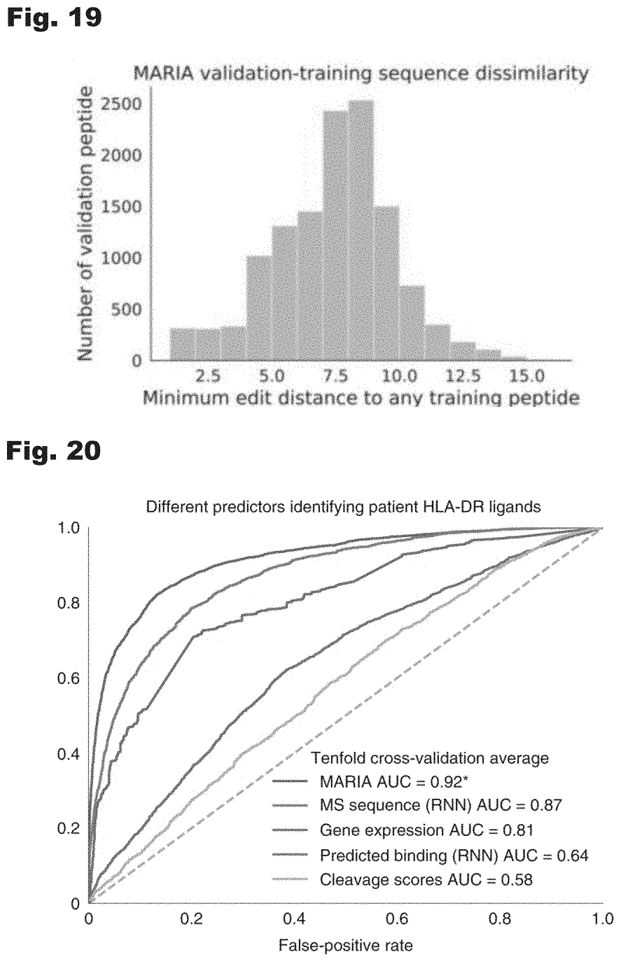

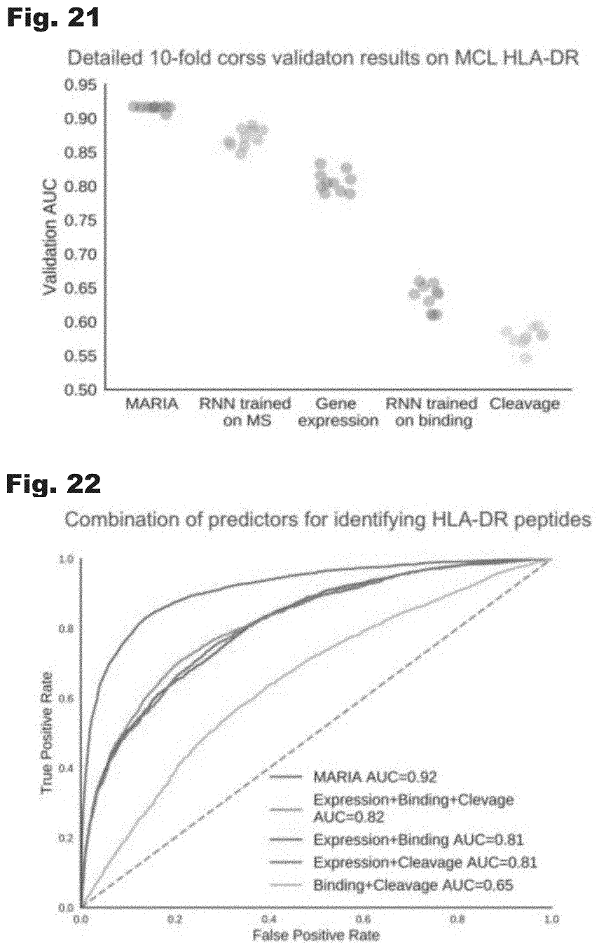

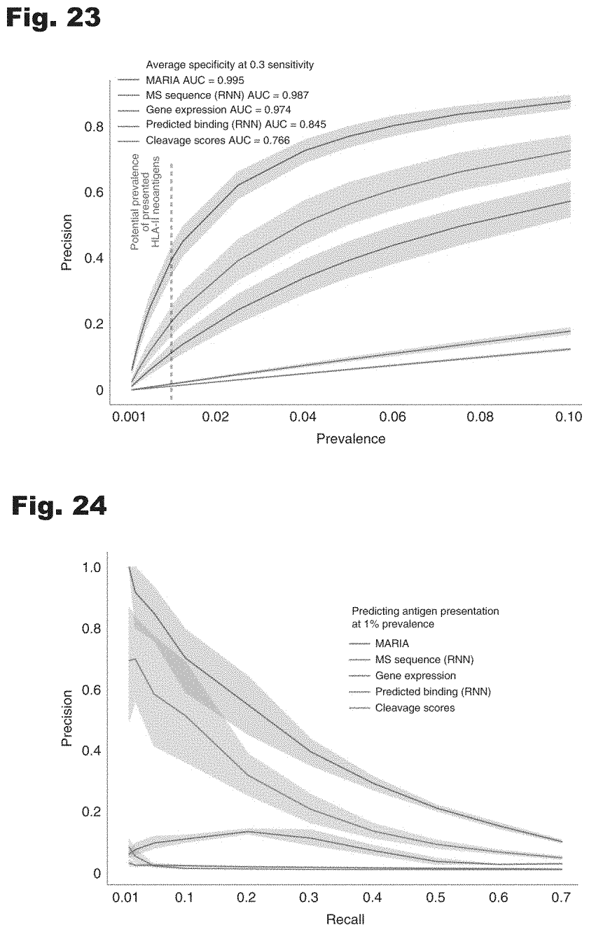

On the basis of the findings above, MARIA was developed with an integrative strategy to better predict HLA-II presentation. The model takes in three input values: the query peptide sequence, the patient or cell HLA-DR allele(s) and the corresponding gene name (FIG. 18). As an intermediate step, MARIA calculates HLA-DR binding scores and cleavage scores using two pretrained neural network models (FIGS. 10A and 10B). Gene expression values are estimated by either tissue-matched external RNA-seq or patient-specific RNA-seq results. MARIA then generates presentation scores for a potential antigen by integrating all available information including peptide sequences with a merge layer (FIG. 18). To process variable length peptide sequence inputs, MARIA includes a recurrent layer with long short-term memory (LSTM). MARIA and models with all possible feature combinations was tested using tenfold cross-validation (FIG. 19). When considering average AUCs, MARIA outperformed an RNN model trained on peptide sequences alone with an AUC value of 0.92 versus 0.87 (FIGS. 20 and 21; P<1×10⁻⁵). By contrast, a logistic regression model trained using binding scores, gene expression levels and cleavage scores achieved a lower AUC value of 0.82 (Supplementary FIG. 22). MARIA provided higher precision (positive predictive values) as compared to alternative models across a broad range of HLA-DR ligand prevalences (0.1-10%; FIG. 23). Assuming 1% prevalence of HLA-II antigens (Supplementary Note 2), MARIA achieved 99.5% specificity and 38.7% precision while identifying 30% of positive peptides (FIGS. 23 and 24).

### MARIA Benchmarking

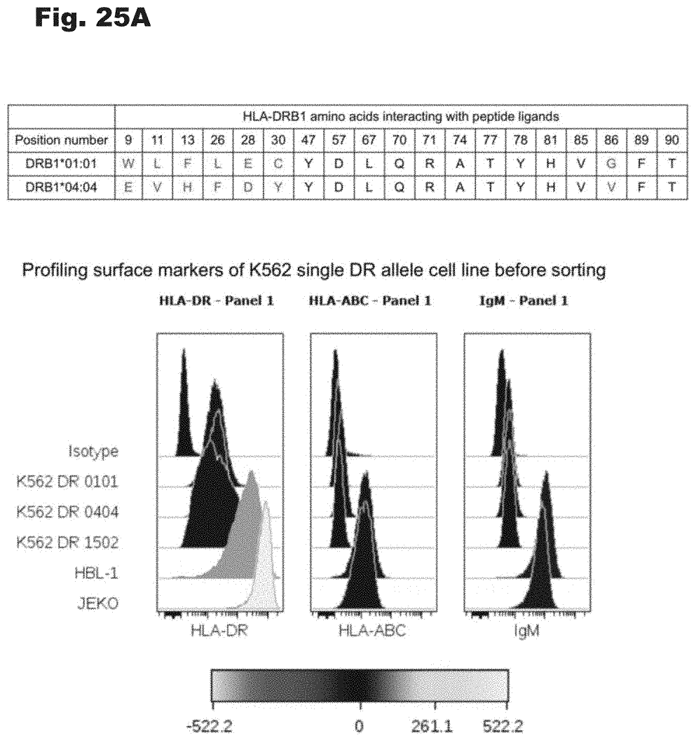

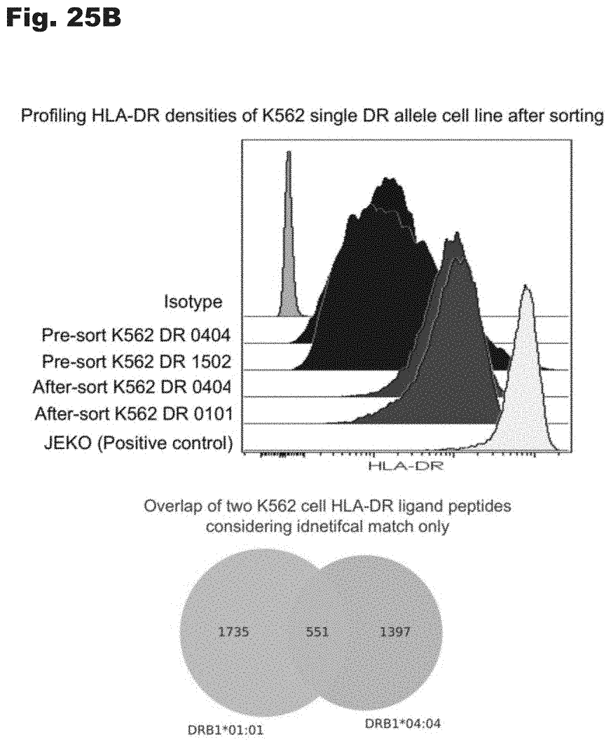

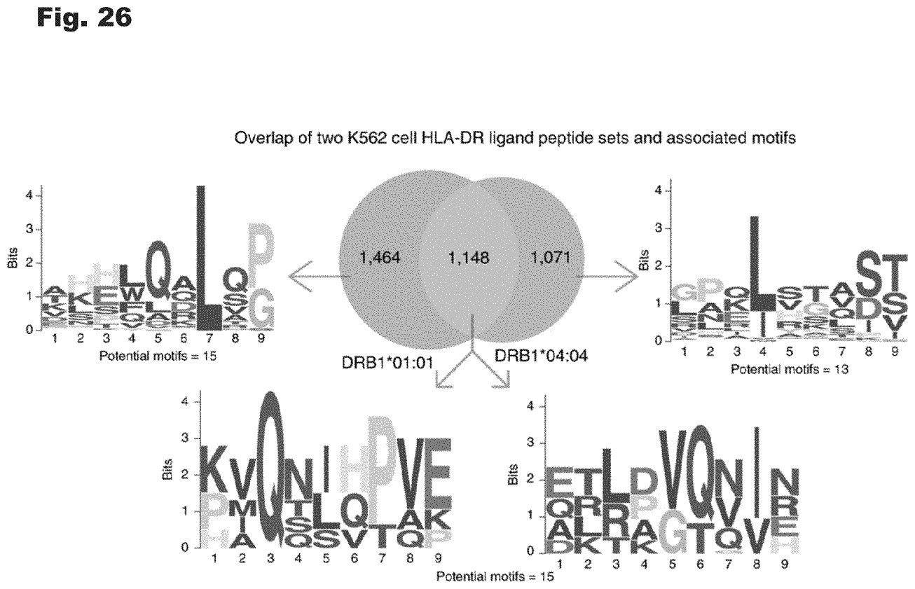

To systematically compare the performance of MARIA with alternative methods, antigen presentation profiling was applied to directly identify HLA-DR ligands from a human cell line (K562) expressing single HLA-DR alleles (DRB1*01:01, DRB1*04:04). Given the myeloid hematopoietic origin of this cell line (in contrast to the lymphoid tumors using for MARIA training), this allowed an assessment of both the effects of HLA-II allelic variation and the cell-of-origin on performance (FIGS. 25A and 25B). Approximately 3,600 non-redundant peptide ligands were identified from these two alleles. When allowing substring matching, ˜31% of ligands were shared (FIG. 26) and MEME identified 15 shared potential binding motifs. This is consistent with known promiscuity of HLA-II binding and presentation.

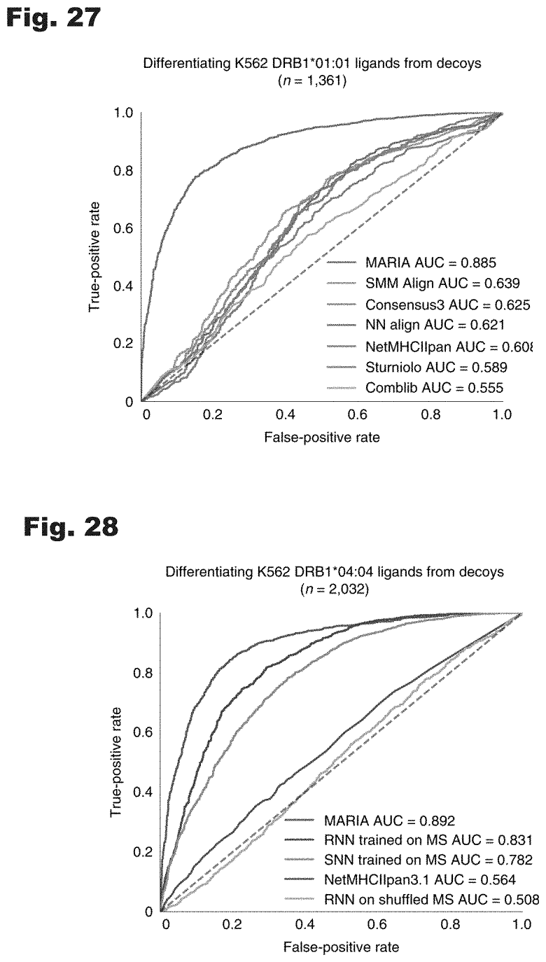

DRB1*01:01 was selected for initial testing in this system, as it has the most abundant training data for existing binding prediction methods, including NetMHCIIpan3.1 (M. Andreatta, et al., Immunogenetics 67, 641-650 (2015)), SMM Align (M. Nielsen, C. Lundegaard, and O. Lund, BMC Bioinformatics 8, 238 (2007)), NN Align (M. Nielsen and O. Lund BMC Bioinformatics 10, 296 (2009)), Sturniolo (T. Sturniolo, et al., Nat. Biotechnol. 17, 555-561 (1999)), CombLib (J. Sidney, et al., Immunome Res. 4, 2 (2008)), and IEDB Consensus3 (P. Wang, et al., BMC Bioinformatics 11, 568 (2010)). The performance of MARIA was benchmarked against these six methods when predicting the presentation of 1,331 DRB1*01:01 ligands empirically and distinguishing them from length-matched decoys (FIG. 27). When compared to the previous MHC-II prediction tools, MARIA outperformed the second-best method (SMM Align) by a significant margin (AUC 0.89 versus 0.64; P<1×10⁻⁵). Performance was also assessed when predicting presentation by HLA-II alleles not present in the training data. Specifically, the HLA-DR ligands were directly profiled from a second monoallelic K562 isogenic line engineered to express HLA-DRB1*04:04, an allele absent from the individuals considered for MARIA training. MARIA again outperformed other methods with an AUC 0.89 (FIG. 28).

The influence of neural network structure on prediction performance was also explored. Using the same MCL dataset of HLA-DR ligands used for training MARIA, a shallow neural network (SNN) similar to NetMHCIIpan with a single hidden layer was trained, as well a deep RNN model. These two models only considered peptide sequences, yet both outperformed NetMHCIIpan on external validation data from K562 (FIG. 28). This is consistent with the hypothesis that directly learning from MS-identified HLA ligands substantially boosts prediction power. Importantly, when trained on the same data, deep neural networks outperformed single-layer architectures (FIG. 28′).

### Extension of MARIA to the HLA-DQ Locus

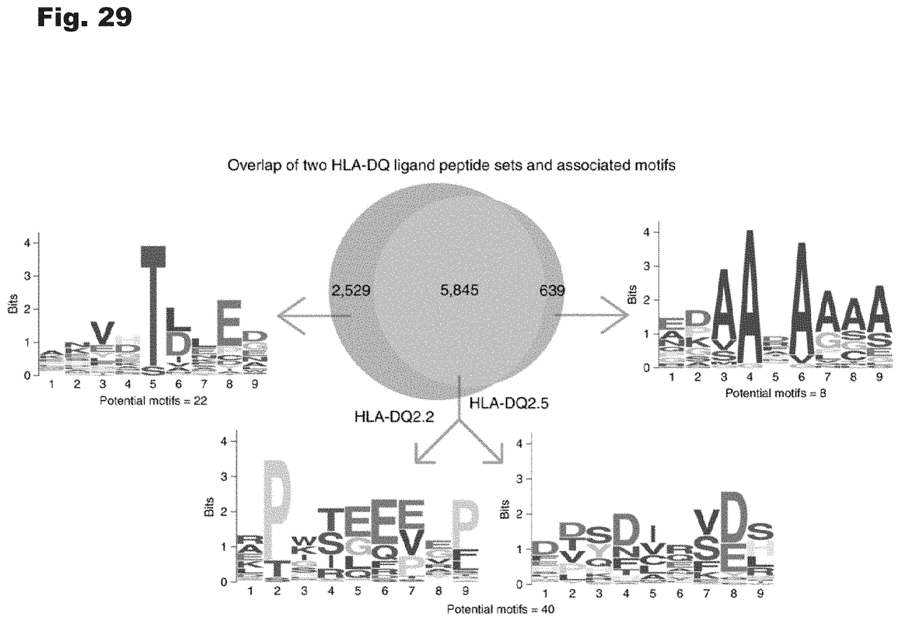

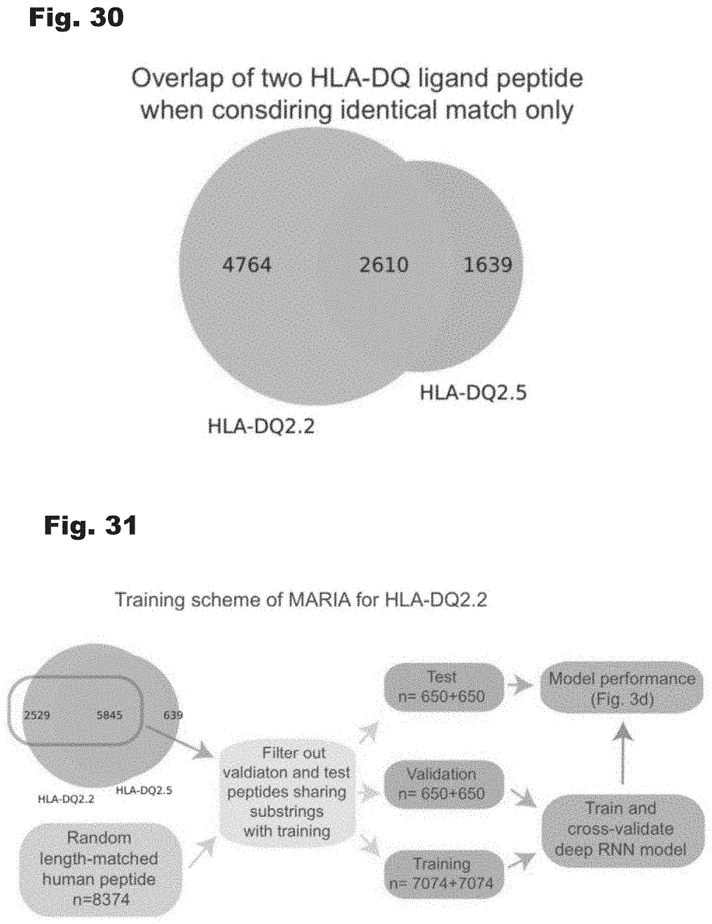

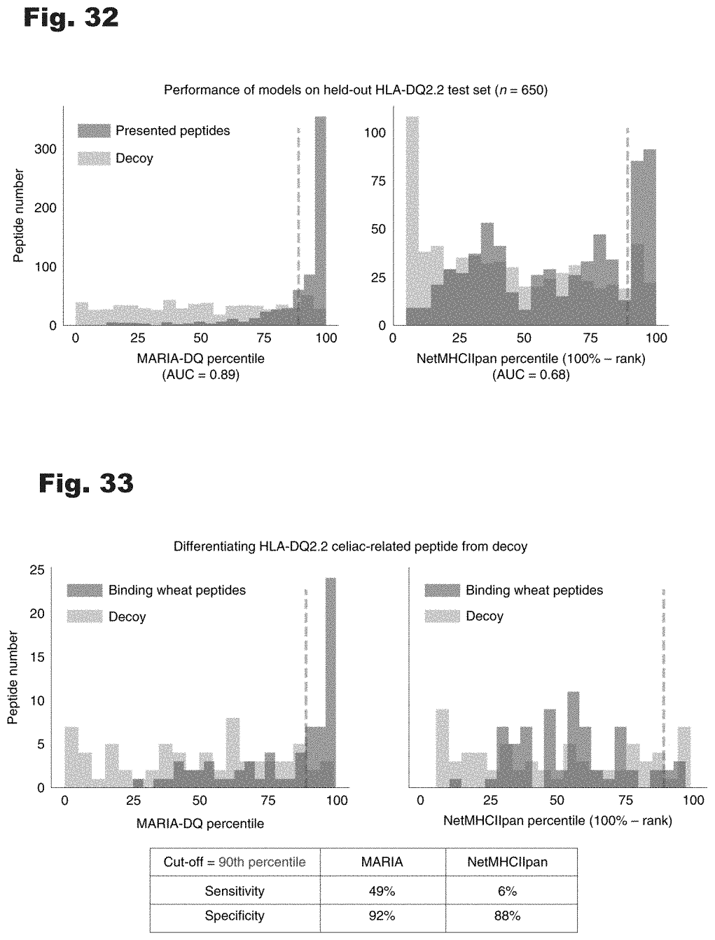

HLA-DQ2.2 (DQA1*02:01 and DQB1*02:02) and HLA-DQ2.5 (DQA1*0501 and DQB1*0201) are known to present wheat gluten peptides and to predispose patients to celiac disease. To test the MARIA prediction framework in the context of the HLA-DQ locus, MARIA was trained on 11,482 HLA-DQ2.2 human peptide ligands identified from previously profiled cell lines (FIGS. 29 and 30). Similar to the HLA-DR alleles profiled above, HLA-DQ2.2 and HLA-DQ2.5 had a large number of shared peptide ligands (65%) and associated sequence motifs (40). After cross-validation (FIG. 31), MARIA was tested on an independent set of 650 held-out human DQ ligands (positives) and 650 length-matched decoys (negatives) and observed an AUC of 0.89 (FIG. 32). To allow comparison between prediction methods on HLA-DQ, both raw MARIA and NetMHCIIpan scores were normalized as percentiles, where higher scores reflect better predicted binding. Within this comparison on an identical test set of HLA-DQ ligands, NetMHCIIpan achieved an AUC of 0.68. Therefore, MARIA provides advantages over existing methods across HLA-II loci.

In addition to the presentation of human peptides described above, the presentation of diverse gluten peptides by DQ2.2 has also previously been profiled by MS (see S. Dorum, et al., J. Immunol. 193, 4497-4506 (2014), the disclosure of which is incorporated herein by reference. Sixty-nine of the wheat peptides presented and 69 decoys were scored with both NetMHCIIpan and MARIA, which was exclusively trained on human peptides. NetMHCIIpan identified 6% of positive gluten peptides with 88% specificity at the recommended cut-off (90th percentile). By comparison, MARIA identified 49% of positive gluten peptides with 92% specificity (FIG. 33) with the same cut-off. MARIA also assigned significantly higher presentation scores to deamidated gluten peptides (FIG. 34), a result which is consistent with increased immunogenicity of gluten peptides upon deamination.

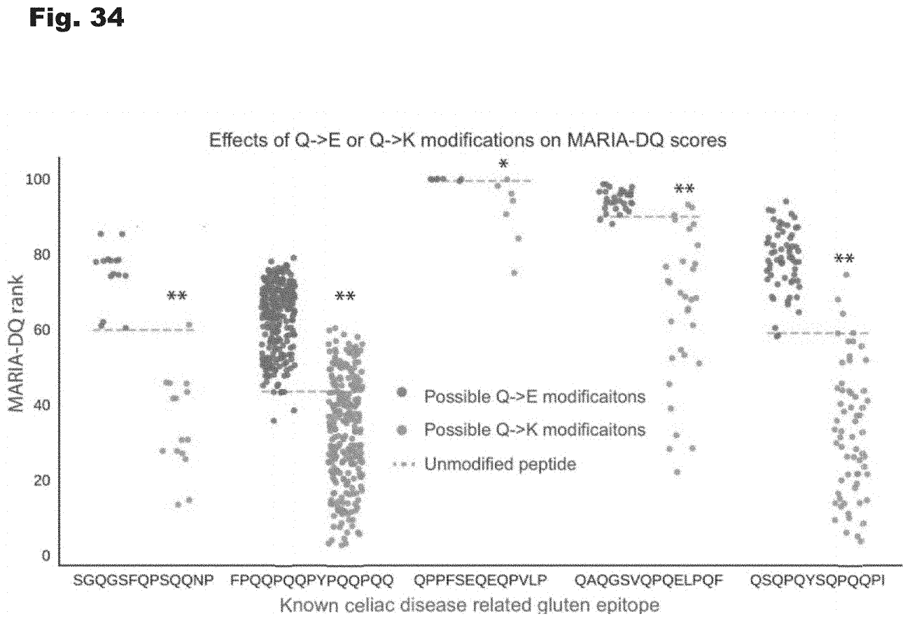

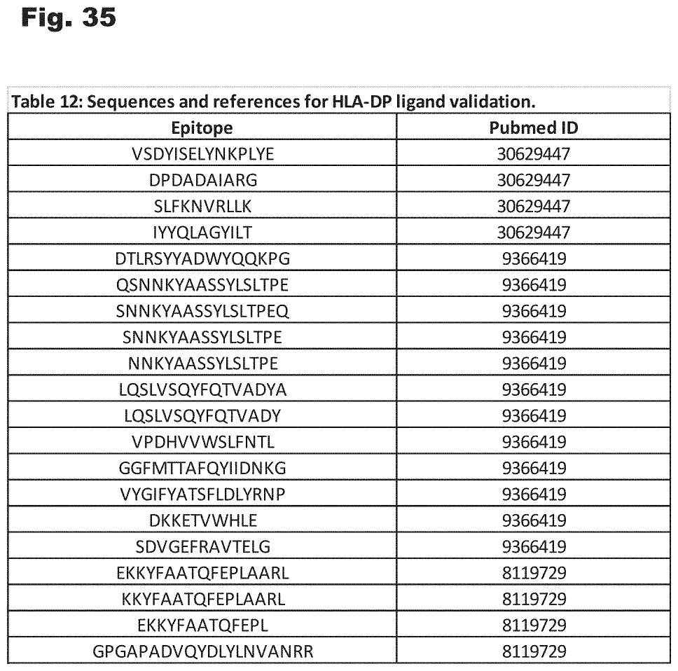

A small number of reported natural HLA-DP ligands were also identified (n=20; Table 12 in FIG. 35) and a new dataset of pan-HLA-II ligands. A pan-HLA-II model with the same framework of the HLA-DQ model was trained and the model demonstrated the utility of MARIA to differentiate ligands from random human peptides (AUC=0.82; FIG. 36). Overall, these results demonstrate that MARIA trained on human peptides can predict presentation of exogenous antigenic peptides by distinct HLA-II alleles.

### MARIA Identifies Diverse Cancer Neoantigens

The ability of MARIA to identify immunogenic neoantigens in cancer was assessed. Ideal antitumor neoantigen candidates should be both presentable by HLA complexes and capable of inducing proinflammatory responses by interacting with T cell receptors. Most current cancer vaccine platforms prioritize candidate neoantigens for vaccine production by selecting only highly expressed candidates with high predicted binding affinity for self-HLA alleles. Yet, many vaccine peptides do not elicit T cell responses upon vaccination, despite rigorous candidate selection. It was therefore tested whether MARIA could better select neoantigens that were most likely to induce a T cell response upon vaccination.

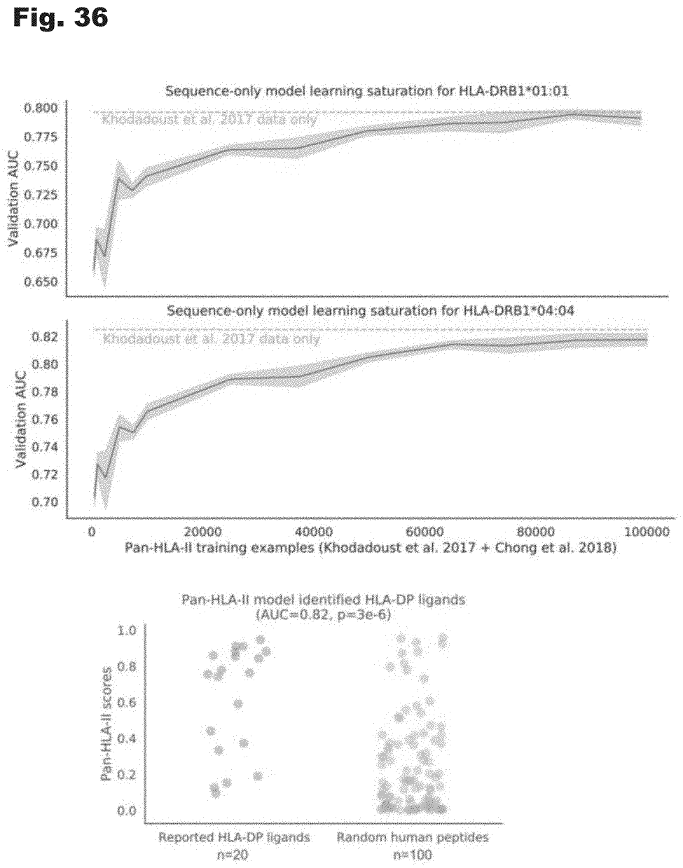

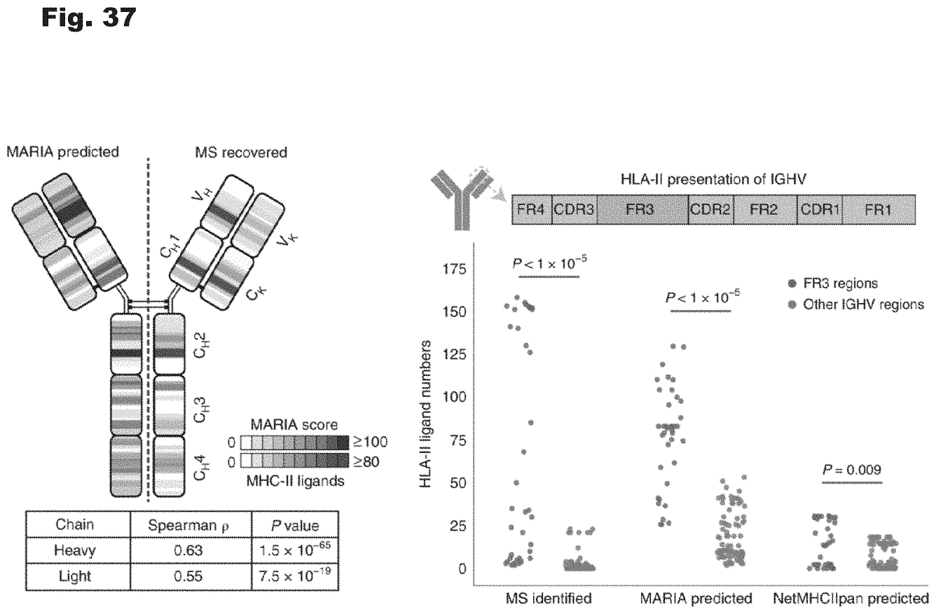

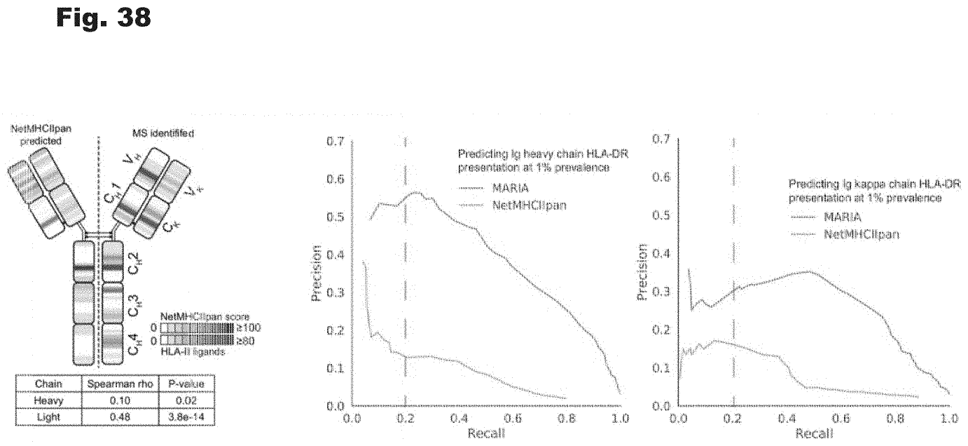

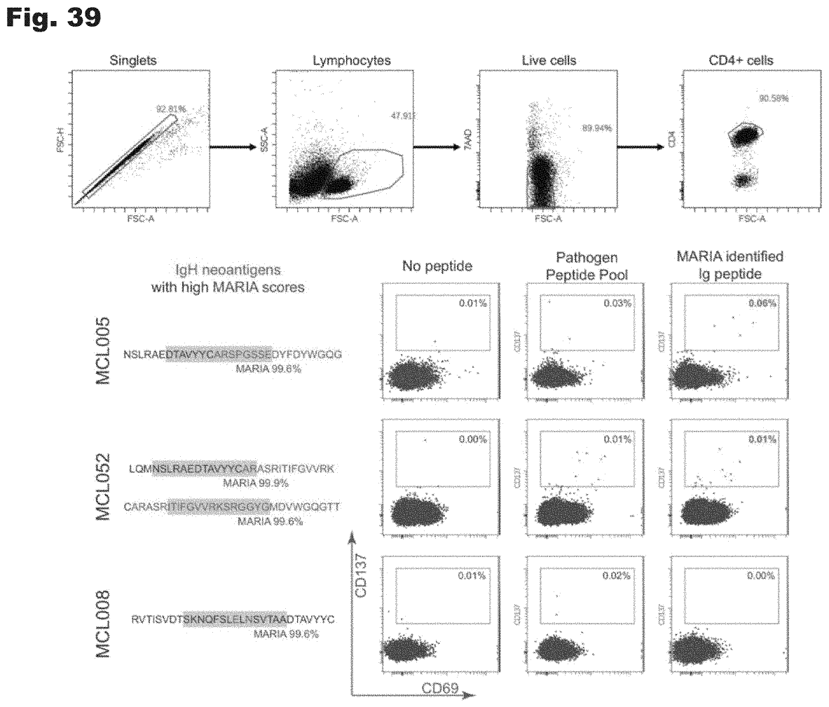

Using antigen presentation profiling, hotspots within specific immunoglobulin (Ig) regions are presented by HLA-DR and associated with antitumor CD4⁺ T cell responses to lymphoma neoantigens. It was therefore tested whether MARIA could accurately identify potential Ig antigens as potential lymphoma-specific targets for immunotherapy. For this test, all Ig-derived peptides were intentionally excluded for the training. This Ig-naive version of MARIA was applied to predict presentation of Ig sequences in the tumors. The resulting MARIA-predicted presentation scores were significantly correlated with MS-identified HLA-DR ligand frequencies across the full-length heavy and light chains (FIG. 37; Spearman's p of 0.65 and 0.55). By comparison, NetMHCIIpan-predicted hotspots had weaker correlation to observed presentation of peptides (Spearman's p of 0.1 and 0.48; FIG. 38). MARIA also outperformed NetMHCIIpan in precision and recall analysis (FIG. 38). Importantly, MARIA identified framework region 3 (FR3) as a presentation hotspot for the heavy chain variable region (FIG. 37; P<1×10⁻⁵). Patient peripheral blood leukocytes were stimulated with the corresponding patient-specific Ig neoantigens identified by MARIA, and measured induction of T cell surface CD137, a validated marker for T cell activation. Stimulation with the Ig neoantigens showed ex vivo CD4⁺ T cell activation in two of three patients (Supplementary FIG. 39).

Immunoglobulin heavy chain variable regions represent challenging test examples as most HLA ligand prediction algorithms including MARIA were trained on wild-type peptides. To further address the utility of MARIA for predicting presentation of mutated peptides, MARIA predictions of HLA-II intrinsic ligand (CLIP) were assessed with and without specific point mutations. For diverse CLIP variants, MARIA scores consistently correlated with stabilizing versus destabilizing structural changes, while NetMHCIIpan did not (Supplementary Note 3 and FIG. 40).

Personalized protein-coding somatic mutations are attractive cancer vaccine candidates in melanoma owing to the high mutation burden of patients with melanoma. MARIA was assessed whether it could prioritize vaccine candidates for melanoma. 10,513 melanoma self-antigens identified by MS were analyzed, generated from two bulk melanoma tumors with distinct HLA-DR alleles (Mel15, DRB1*03:01 and DRB1*07:01; Mel16, DRB1*13:01 and DRB1*08:03). Each melanoma-presented ligand or decoy was scored using both NetMHCIIpan and MARIA (trained on lymphoma data). Even without patient-specific gene expression data, MARIA outperformed NetMHCIIpan when differentiating melanoma HLA-II ligands from decoys (FIG. 41; AUC of 0.89 versus 0.64; P<1×10⁻⁵).

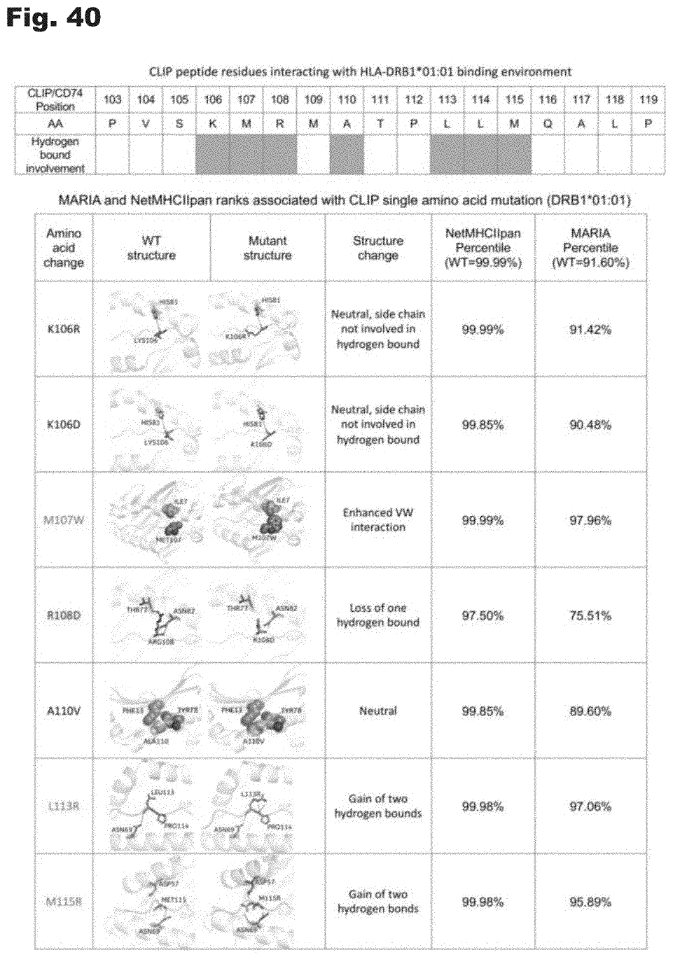

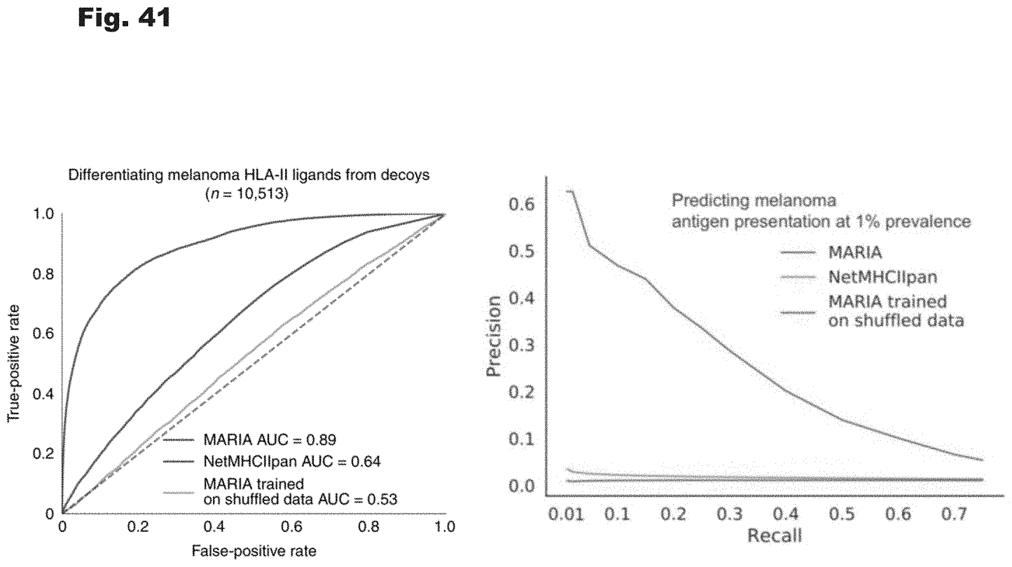

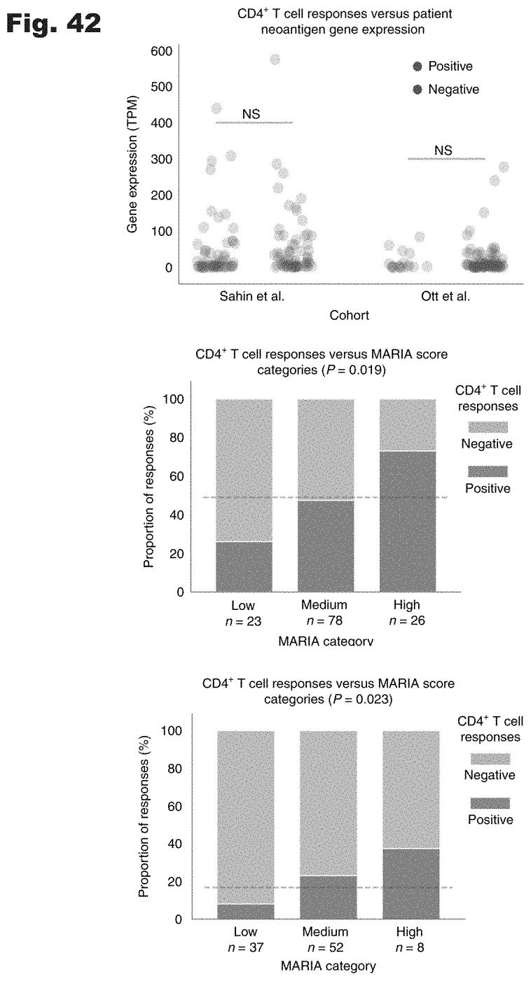

After confirming the performance of MARIA in non-hematopoietic tissue, such as melanoma, MARIA was used to analyze two sets of personalized melanoma vaccine neoantigens with corresponding immune response data (ex vivo CD4⁺ T ell enzyme-linked immunospot (ELISPOT) test; n=121 and 96). Gene expression levels of neoantigens alone for T cell reactive and non-reactive neoantigen candidates were largely indistinguishable (FIG. 42; P>0.4). MARIA assigned each peptide a percentile score by comparing the raw score to scores of 20,000 random human peptides as described. In each of these independent cohorts, the majority of selected neoantigens for vaccination (81% and 62.5%) were scored in the 95th percentile or above of MARIA scores (FIG. 42), consistent with the authors' attempts to select the best HLA binders. Neoantigens with lower than 95th percentile MARIA scores (FIG. 42) were less likely to successfully induce a T cell response upon vaccination. Specifically, only 26% and 8.3% of such neoantigens resulted in successful ex vivo CD4⁺ T cell responses upon vaccination.

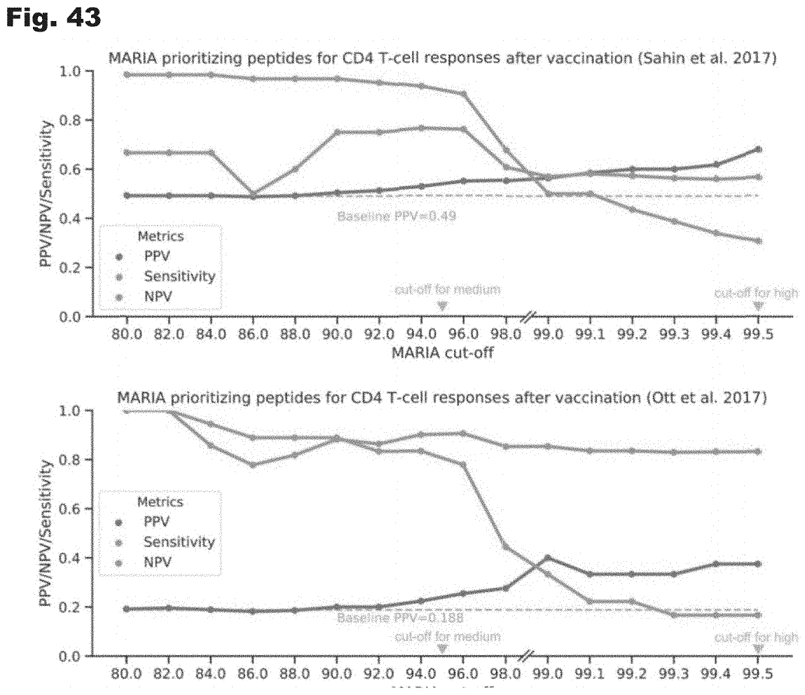

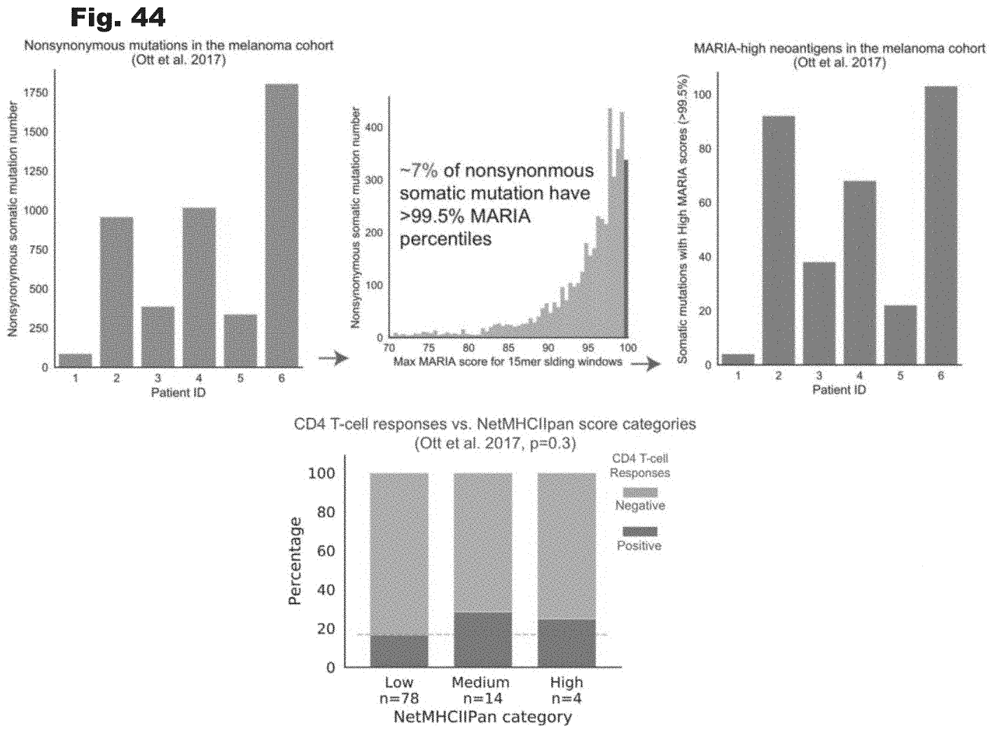

Conversely, peptides with the highest MARIA scores (>99.5% MARIA percentiles) were more likely to elicit a T cell response upon vaccination (FIGS. 42; 73% and 38%; P=0.019 and P=0.023). This stringent MARIA cut-off achieved a high positive predictive value (PPV) in both trials, and MARIA showed higher than baseline PPVs across a range of cut-offs (FIG. 43). Of note, ˜7% of all melanoma somatic mutations scored higher than 99.5% MARIA percentiles (FIG. 44), suggesting the availability of many more vaccine candidates for effective immunization than were tested. With HLA-DR alleles available in the same trial, each candidate was also scored with NetMHCIIpan. NetMHCIIpan scores were weakly correlated with T cell responses but did not reach statistical significance (FIG. 44).

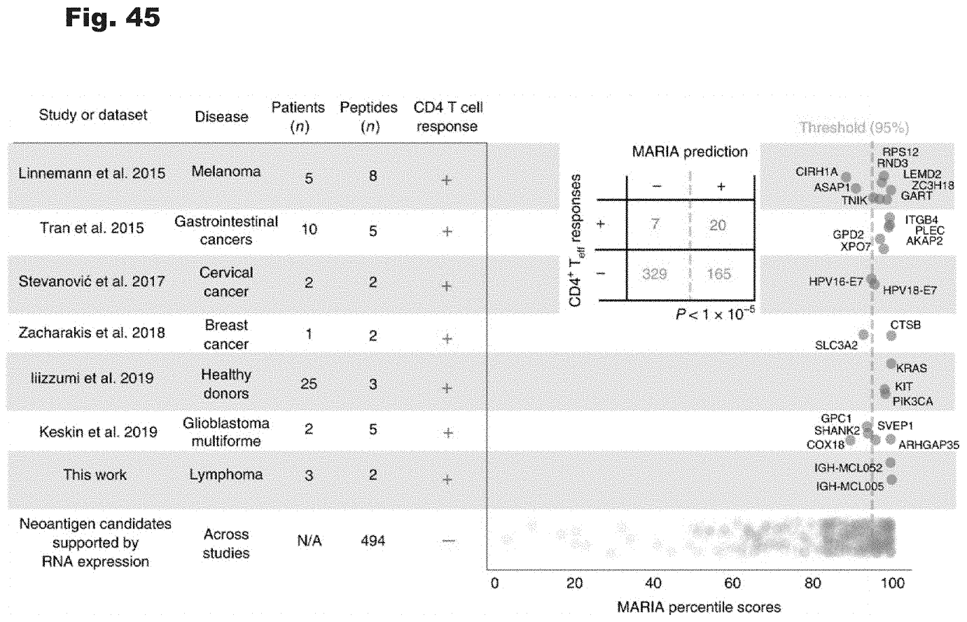

To further validate MARIA performance for predicting immune responses, seven additional cancer-related CD4⁺ T cell response studies were assessed. Each of these studies individually identified a small number of cancer-associated CD4⁺ T cell epitopes across diverse cancer types. Using a 95% MARIA percentile cut-off (FIG. 42), 74% of CD4 epitopes were identified with 67% specificity (FIG. 45). Therefore, while MARIA was not trained on T cell response data, MARIA scores show promise for prioritizing HLA-II neoantigens most likely to induce corresponding CD4⁺ T cell responses.

### MARIA Results Summary

HLA-I and HLA-II both play central roles in antigen recognition and adaptive immune responses. HLA-II gene expression analysis in the Cancer Genomic Atlas (TCGA) cohorts suggests abundance of antigen-presenting cells or tumor HLA-II presentation in various cancer types (FIG. 46). Historically, HLA-I ligand prediction algorithms have superior performance as compared to HLA-II. Recent studies in HLA-I have shown that prediction accuracy can be improved by learning directly from naturally identified ligands and considering non-sequence features. However, the variable length of HLA-II peptide ligands as well as the heterogeneity of other useful features have made translating the same framework for HLA-II challenging within conventional neural networks. MARIA represents a tool to tackle these two challenges by using multimodal RNNs, which are capable of integrating heterogeneous features and variable length sequences. The results suggest that using deep learning methods are superior to shallow neural networks (SNNs) for HLA-II prediction (FIG. 28). This is likely due to the ability of RNNs to consider multiple binding motifs, as SNNs typically rely on a single nine-amino-acid binding core.

The SysteMHC Atlas was recently constructed to consolidate HLA-I and HLA-II ligand sequences from a diverse set of studies. MARIA was designed to be capable of integrating additional training from emerging sources such as the SysteMHC Atlas and expect its performance to improve as such ligand datasets continue to grow. For example, recurrent patterns in HLA-II cleavage signatures was observed, including the enrichment of ligand flanking sequences for tyrosines and their depletion for prolines. However, cleavage signatures from different cell types showed subtle variation in motif sequences (FIGS. 17B and 17C). Accordingly, additional HLA-II ligand data in the SysteMHC Atlas can provide a window to systematically investigate cleavage signatures in each cell type, thus allowing better MARIA predictions for distinct tissues. Separately, as MARIA was not trained on presented non-human peptides, emerging microbial datasets can be used to further refine MARIA for predictions relevant to infectious disease.

The results also demonstrate how MARIA might allow researchers to better identify immunogens relevant to autoimmunity and to antitumor immunity. Given the inherent challenges limiting the accuracy of previous methods for characterizing tumor-derived HLA-II ligands, MARIA should allow researchers to explore less well-studied HLA-II neoantigens. Specifically, MARIA is useful for directly identifying and prioritizing cancer vaccine candidates from patient sequencing data.

### In Vitro Binding Testing for Validation of HLA-DR Binding

Candidate peptides were synthesized with N-terminal 2,4-dinitrophenyl (DNP) tags joined by a 6-am inohexanoic acid linker (Sigma). Biotinylated HLA-DR recombinant proteins (HLA-DRB1*04:03 and HLA-DRB1*07:01) molecules were provided by the NIH tetramer core. Intrinsic CLIP peptide was cleaved from the HLA-DR molecules with human rhinovirus 3C protease. DNP-tagged peptides were supplied in molar excess to encourage efficient exchange of binders and incubated overnight at 32° C. or 37° C. (pH 4.5). Exchange reactions were then neutralized with 1 M Tris, pH 8.0 and biotinylated HLA-DR molecules were bound to streptavidin microspheres (Polyscience). Microspheres were washed and stained with allophycocyanin (APC)-labeled anti-HLA-DR (clone L243; BD Biosciences, 340549) and anti-DNP (clone 2-9(4); Abcam, ab6306) followed by rat anti-mouse IgE FITC secondary antibody (clone R35-72; BD Biosciences, 553415). Microspheres that were positive for HLA-DR and DNP-tagged peptide were detected by flow cytometry. Peptides were considered to be binders if both HLA-DR and DNP signals were detectable above an HLA-DR unexchanged control. FIG. 7 shows full benchmarking with reported binders and non-binders.

### Development of K562 Cells Expressing Single HLA-DRB1 Alleles

Cell lines expressing single HLA-DR alleles were prepared from K562 cells, which do not express surface class I or II HLA, by lentiviral transduction. Sequences for the DR α-chain and the relevant β-chain alleles (DRB1*01:01 and DRB1*04:04) separated by a 2A peptide sequence were encoded in the N103 lentiviral vector backbone (kindly provided by J. Crabtree, Stanford University) and used to produce lentiviruses in HEK293 cells. To enhance expression levels of HLA-DR in the K562 cell lines, the top 1% of cell populations were selected and expanded on the basis of surface HLA-DR signal with fluorescence-activated cell sorting (clone L243; BD Biosciences 347367). Expression of HLA-DR was confirmed by flow cytometry before and after sorting (FIGS. 25A and 25B). K562 cells were also monitored for surface HLA-I alleles to ensure no endogenous HLA expression was present (anti-HLA-I antibody; clone G46-2.6; BD Biosciences, 555555). Cells were maintained in DMEM medium (Sigma) supplemented with 2.0 μg m1⁻¹ puromycin (Sigma).

### Identification of K562 HLA-DR Ligands

HLA-DR immunopeptidomes were extracted from the K562 HLA-DRB1*01:01 and K562 HLA-DRB1*04:04 cell lines. HLA-DR molecules were isolated and the associated peptides were extracted. See Supplementary Note 4 for detailed HLA-DR immunopeptidome purification and MS analysis.

### HLA-II Ligand Sequence Data Sources

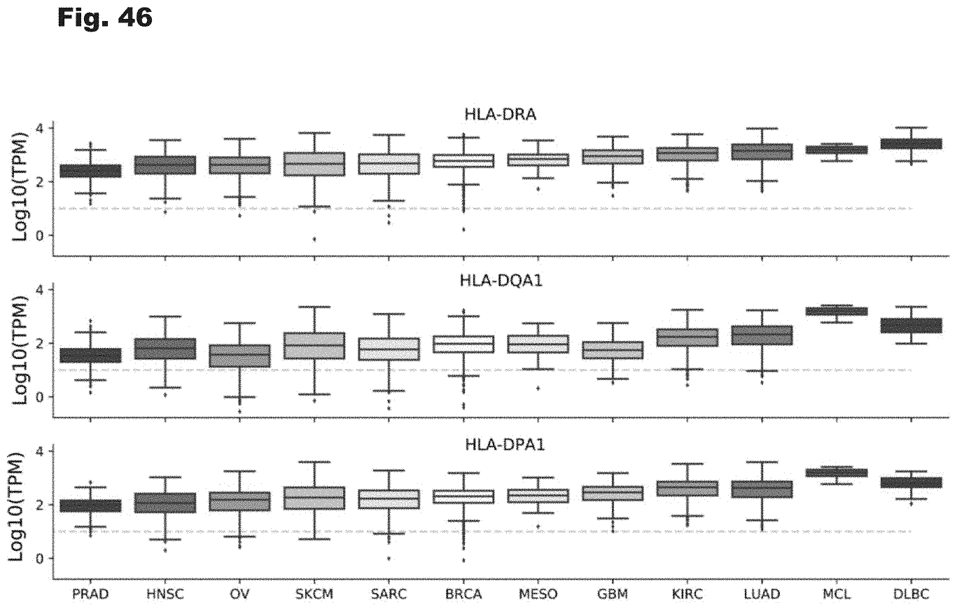

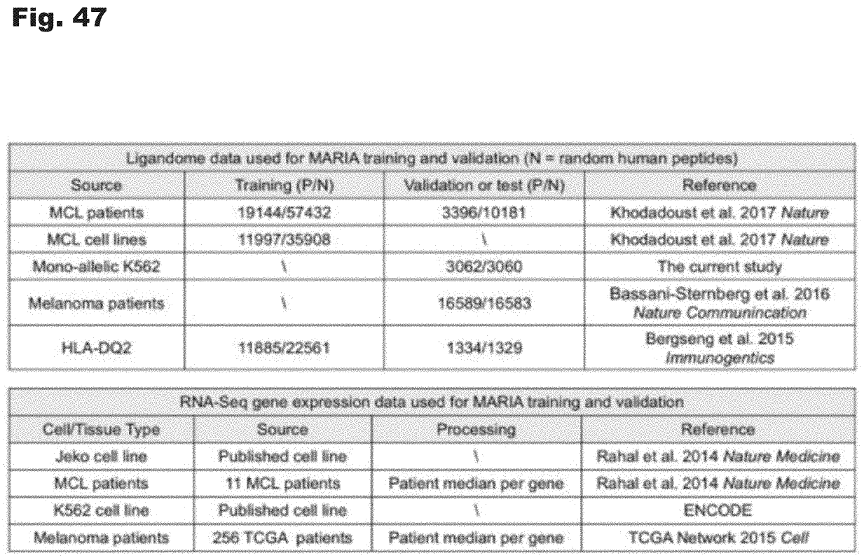

Detailed ligand sequence data sources are listed in FIG. 47. MCL HLA-DR ligandomes were obtained from previous studies. Dendritic cell HLA-DR ligandomes were obtained from a MUTZ3 cell line study. HLA-DQ2.2 ligandomes were obtained from a monoallelic B cell line study conducted with the anti-DQ antibody SPV-L3. Melanoma HLA-II ligand sequences were obtained from a previous study on primary tissues from patients with melanoma. HLA-DQ2.2-presented wheat peptides were downloaded from the IEDB database. Pan-HLA-II ligands were obtained from a study of B cell lines and ovarian carcinoma using HB-145 anti-HLA-II antibody. Monoallelic HLA-I ligand sequences were obtained from a B cell line study with W6/32 pan-HLA-I antibody. HLA-DP ligands were downloaded from the IEDB database.

### Determination of HLA Alleles

HLA alleles of patients with MCL were identified with PHLAT from patient tumor exome sequencing data. HLA alleles of melanoma patients were identified with HLAVBSeq from patient exome sequencing data. When patient alleles were not available, HLA-DRB1*07:01 and HLA-DRB1*01:01 were used as they are the most common alleles in general populations (www.allelefrequencies.net/).

### Immunogenicity Testing for Immunoglobulin Neoantigens

All specimens were obtained with informed consent in accordance with the Declaration of Helsinki and this study was approved by Stanford University's Administrative Panels on Human Subjects in Medical Research. Samples were collected from patients as part of a clinical trial of autologous tumor vaccination (NCT00490529). Patient leukocytes were collected by leukapheresis approximately 2 weeks after a series of autologous tumor vaccinations. Cells were cultured in a 1:1 mix of AIM-V medium and RPMI1640 (Thermo Fisher) with 10% pooled human AB sera (Gemini Bio) and 50 μM β-mercaptoethanol. Neoantigen peptides were synthesized (ElimBio) and added to a final concentration of 10 μg m1⁻¹. In one patient (MCL052), cells were treated concurrently with two predicted neoantigen peptides. As a positive control, cells were stimulated with a mixture of pathogen-associated peptides, CEFT pool (JPT Peptide Technologies). Cells were incubated for 30 h before flow cytometry analysis. CD137 (clone 4B4-1; BD Biosciences, 561702) and CD69 (clone L78; BD Biosciences 341652) expression was assessed on live CD4⁺ (clone RPA-T4; BD Biosciences, 562659) T cells using a FACS Aria sorter (BD Biosciences). See FIG. 39 for gating strategies.

### Gene Expression Data Sources

Only minor differences in gene expression profiles were observed when using personalized versus inferred gene expression levels with modest impacts on MARIA prediction results (FIG. 22 and Supplementary Note 1). Therefore, when personalized gene expression profiles were not available, expression profiles were estimated from the corresponding tumor type, using, for example, the median of TCGA RNA-seq results from the closest tissue type. Gene expression profile of patients with MCL and JeKo-1 cell line (MCL origin) were obtained from RNA-seq results; MCL patient gene expression profiles were estimated as the median value across ten patients with MCL. Given the high correlation when comparing MCL transcriptomes from different tumors, gene expression profiles of the L128 cell line (MCL origin) were estimated from JeKo-1 cell line RNA-seq values. The gene expression profile of K562 cell lines was obtained from the ENCODE database. Expression values were normalized into TPM to enable direct comparison of values across studies.

Gene expression profiles of melanoma in the cancer vaccine studies were provided when analyzing how original neoantigen gene expressions correlated with responses. A fixed TPM of 50 was used as input for MARIA to evaluate how MARIA scores with post-vaccine responses as cancer vaccines made each neoantigen peptide readily available to antigen-presenting cells. A fixed TPM of 50 was also used when evaluating how HLA-DRB1*01:01 binds to CLIP. Gene expression values were not needed when MARIA analyzed HLA-DQ2.2 or HLA-DP peptides, as only peptide sequences were used during training.

### Gene Expression Analysis of HLA-II Peptides

The gene expression value (TPM) of each HLA-DR-peptide-producing gene was estimated with RNA-seq of samples from patients with MCL or MCL cell lines. A gene was allowed to be counted multiple times if multiple peptides were identified from the same gene. Each gene expression value was converted into logarithmic space with log₁₀(X+10⁻⁶) and represented as a violin plot (FIGS. 12 to 15). Peptide sequences not in the dictionary or genes with unknown RNA-seq gene expression values were excluded from this analysis (<8%).

Using a similar approach, the gene expression profiles of all genes were analyzed in patients with MCL and MCL cell lines as a background distribution. Each gene with a known RNA-seq gene expression value was converted into logarithmic space, and each gene was counted once. The median was calculated and a Mann-Whitney U test was conducted on gene expressions between MCL HLA-DR-presented genes and the background distribution.

To investigate sources of MCL HLA-DR peptides with extremely low gene expression values, Gene Ontology (GO) term enrichment analysis was conducted on HLA-DR peptide genes with TPM <0.1 (FIG. 12). To correct for the presence of extracellular matrix protein and blood protein inside of professional antigen-presenting cells (for example, MCL) when these genes were not endogenously expressed, gene expression values of genes under the following GO terms were set to 50 TPM: extracellular matrix (0031012), blood microparticle (0072562) and secretory granule lumen (0034774).

To test the ability of gene expression values to differentiate HLA-II ligands from decoys, a logistic regression model was built with gene expression values as a single feature to differentiate HLA-DR peptides from a random human peptide decoy. Decoy gene lists were generated from a Uniport reviewed human protein list and were enriched for expressed genes. Specifically, human peptide decoys contain roughly 40% genes with TPM >10, 10% genes with unknown expression levels and 50% genes with TPM <10. Predictive performance of gene expression values under different assumptions is shown in FIG. 14. No regularization was applied because it is a single-feature model.

### Neural Network Implementation

See Supplementary Note 5 for an overview of neural networks considered and their implementation. Neural networks in this study were implemented with Keras 2.0.3 (keras.io/) using the Tensorflow backend and Python 2.7. For training, an NVIDIA Tesla K80 GPU with CUDA 7.5.18 was utilized on the Stanford XStream GUP cluster to speed up the gradient descent.

### Amino Acid Representation

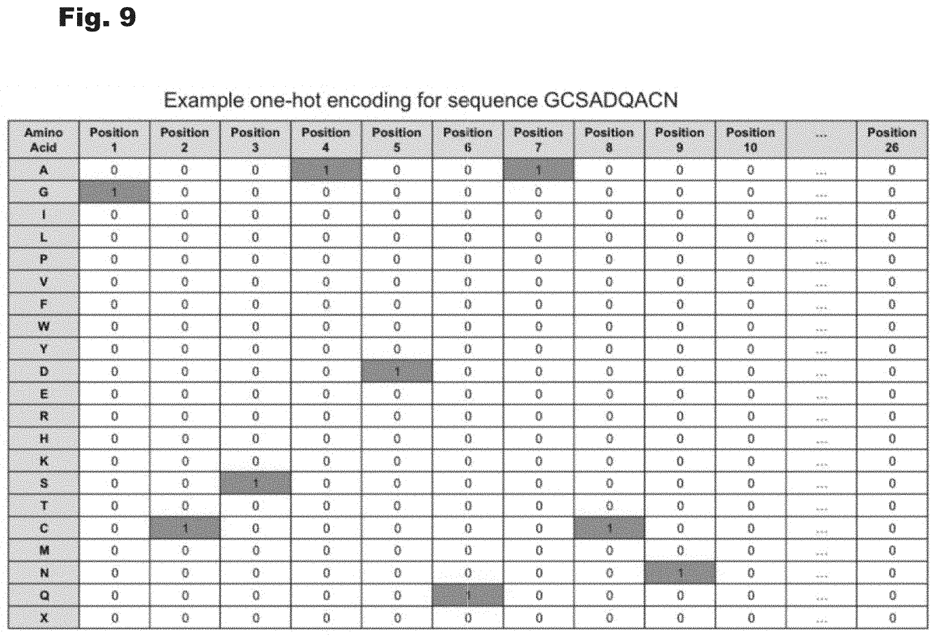

Each amino acid in a peptide sequence is represented by a 21-number one-hot vector (20 common amino acids+X) A one-hot vector is a vector of zeros with a single one that indicates the amino acid species in an arbitrary but consistent mapping (for example, position one=alanine; FIG. 9). Thus each peptide sequence is represented by a (21, L) two-dimensional vector, where L is the length of the peptide. BLOSUM50 and ProtVec amino acid encodings were attempted but had little influence on the model performance.

### MARIA Model

The majority of existing HLA-II peptide prediction algorithms use data on the binding of recombinant HLA-II protein to peptides as a surrogate to rank potential HLA-II peptides presentation or HLA-II neoantigens. MARIA aims to directly predict the probability of a given peptide being presented by a HLA-II complex in a cell or patient context. Rather than in vitro measurements of the binding of HLA protein to peptides, HLA-II ligands identified by MS/MS peptide sequencing data from antigen-presenting cells were used as the ground truth.

The MARIA model takes peptide sequences (8-26 amino acids long), patient or cell HLA-DR alleles and corresponding gene names to predict peptide presentation by the HLA-DR complex (FIG. 18). MARIA estimates HLA peptide-binding affinities and cleavage scores with two separate neural network models, which are described in the next sections. MARIA also estimates the gene expression levels of each input gene with a gene expression profile dictionary that is based on external tissue-matched RNA-seq results (gene name and TPM). The MARIA model takes in gene expression values (TPM) as log₁₀(TPM+0.001) to prevent neurons that connect to gene expression input from dominating the optimizing gradient. Users can also upload their own gene expression profile dictionaries for each query. The influence of using patient-matched or external gene expression profile dictionaries are explored in FIGS. 14 and 15.

Besides estimating binding affinities, cleavage scores and gene expression values, MARIA encodes each ligand peptide sequence using an RNN layer known as LSTM. All of these values are integrated with two fully connected dense layers to estimate the probability of a peptide-gene pair being presented in a allele- and gene-expression-specific manner (FIG. 18). See Supplementary Note 6 for models that predict presentation with peptide sequences only.

LSTM networks with 32, 64 and 128 neurons were assessed and from one to four layers deep with a 9:1 training: validation scheme (FIG. 10B). An LSTM network with 64 neurons and one layer of depth gave the best performance. Dense (fixed-length) layers in MARIA use the rectified linear unit activation function and 32 neurons. Dropout of 40% is applied to each layer for regularization. L1 and L2 regularization were attempted but not included in the final model owing to the lack of influence on the model performance. The LSTM and dense layers were concatenated to merge their information, followed by two additional dense layers (neuron number=32). The output layer contains two neurons representing non-presenting (F or 0) and presenting (T or 1) classes.

MARIA was trained with the Tensorflow GPU backend to enable parallel calculation of gradient. For general user applications, the CPU backend is sufficient. Ten thousand peptides take ˜80 s with a 2.8 GHz Intel Core Xeon CPU or 11.3 s with one NVIDIA Tesla K80 GPU.

### Binding Affinity Prediction

MARIA assumes the main influence on HLA-DR allele is from the change in HLA-DR-peptide binding affinity. A pan-allele regression model was trained with the publicly available IEDB data to estimate binding affinity given a peptide-allele pair. Training data was curated with 33,909 peptide-allele pairs for HLA-DR. Each HLA-DR allele is converted into a 19-amino-acid pseudopeptide sequence reflecting 19 amino acid residues in HLA-DRB1 interacting with ligand peptides in the binding groove. Each peptide-allele pair has a corresponding binding affinity measured in half maximal inhibitory concentration (IC₅₀, in nM). For computational efficiency, IC₅₀ was converted into log space with the formula (1−log_{50,000}(IC₅₀)). The model includes an LSTM layer followed by two dense layers and a single output neuron (FIG. 14). Mean squared error is used as the loss function.

To evaluate the performance of the LSTM-based binding affinity predictor with NetMHCIIpan3.1, MARIA was evaluated on the same held-out set of in vitro binding. Similar ROC-AUC scores and Pearson's correlation coefficients (predicted versus measured) were revealed.

### Cleavage Score Estimation

To understand amino acid preference for HLA-II peptide cleavage, amino acid frequency around HLA-II peptide cleavage sites was compared with a background distribution. Cleavage sites included six amino acids upstream of the HLA-II peptide N terminus (−6 to −1) and six amino acids downstream of the HLA-II peptide C terminus (+1 to +6) assuming a N terminus to C terminus direction. An equal number of gene-matched and length-matched peptides was randomly generated on the basis of the HLA-II peptides, and the amino acid frequency from the same cleavage sites was used as the background distribution. Enrichment and depletion level were calculated as HLA-II peptide amino acid i frequency at j position divided by background distribution amino acid i frequency at j position (FIGS. 16 and 17A-17C). The analysis was done for MHC-I as well (FIG. 17A).

A neural network was built to quantitatively estimate cleavage scores given the flanking regions of a query gene-peptide pair (FIG. 10A). The model determines six amino acids upstream of the query peptide N terminus and six amino acids downstream of the query peptide C terminus with a human proteome dictionary, encodes these 12 amino acid sequences, processes them with hidden layers and outputs a probability score between 0 and 1 (score of the positive output neuron T). The neural network encodes amino acids using one-hot encoding as described before and contains two fully connected hidden layers of 32 neurons. To avoid overfitting when applying this model to lymphoma data, the cleavage model was trained on an independent dendritic cell line (MUTZ3) ligandome (FIG. 17B).

### Normalization of MARIA Output Scores to Percentiles

Raw output of MARIA is a score between 0 and 1 indicating how likely a query peptide is to be presented by a specific HLA-II. To increase human interpretability and enable comparison across different peptide lengths, MARIA's output can be represented as a percentile score. A percentile score is generated by comparing the raw output score to a score distribution generated from length-matched random human peptides. The higher the percentile, the more likely the peptide will be presented by a cell HLA-DR complex.

Specifically, for each peptide length of 8 to 26, 20,000 random natural peptides were sampled from the human proteome. MARIA was run on each set of random peptides and used the output as empirical distributions for normalizing query peptides with a certain length. For example, MARIA output of a 15-amino-acid query peptide will be compared against the scores of 20,000 random 15-amino acid oligomers to obtain a percentile score. NetMHCIIpan used a similar approach to generate ranks, and NetMHCIIpan percentiles=100%−NetMHCIIpan ranks.

### MARIA Model Evaluation on Held-Out HLA-II Ligand Sets

To fully evaluate the performance of MARIA and related methods, a set of independent HLA-DR ligandomes was obtained from various cell types and MS instruments (FIG. 26). Any peptides shorter than 8 amino acids or longer than 26 amino acids were excluded (<1%) owing to the setup of the RNN. Because of input limitations of existing methods, peptides with certain length were excluded. When the evaluation involved IEDB-carried methods, peptides shorter than 15 amino acids were excluded. When the evaluation involved NetMHCIIpan3.1, peptides shorter than 9 amino acids were excluded. For negative examples, length-matched random human peptides with 1:3 (training) or 1:1 (validation) positive:negative ratios were used (FIG. 47). During cross-validation, any peptides in the validation set that were substrings (A is part of B or B is part of A) of any training peptides were excluded (FIGS. 19 and 23).

### Benchmarking of Binding-Based Methods on MS-Identified HLA-DR Ligands

To evaluate how NetMHCIIpan performed on MS-identified MCL HLA-DR ligands, NetMHCIIpan3.1 was run on a set of 18 MCL samples with a minimum of 200 peptides identified (Table 1 in FIG. 3). For a patient or cell with heterozygous HLA-DRB1 alleles, the better binding score (lower ranking or higher percentile) was used as the predicted score. ROC-AUC scores were calculated to evaluate predictive performance of binding models on naturally presented peptides.

MARIA was benchmarked with six commonly used HLA-II prediction algorithms on non-MCL data: NetMHCIIpan3.1, SMM Align, NN Align, Sturniolo/TEPITOPE, Comblib and IEDB Consensus. All six algorithms were trained on in vitro recombinant protein binding data but differ in allele coverage and machine learning algorithms. Held-out data included K562 myeloid cells and primary melanoma patient samples. Because IEDB Concensus (Concensus3) is only compatible with 50 HLA-DR alleles and peptide sequences longer than 14, MARIA was compared with NetMHCIIpan in most cases.

NetMHCIIpan and NN align both use a dense neural network to scan through a given query peptide with a 9-amino-acid oligomer sliding window. The best 9-amino-acid oligomer score is reported. NetMHCIIpan incorporates important amino acid sequences on HLA-II alleles as a part of input features to train a universal algorithm for all HLA-II alleles. NN Align learns binding patterns of each HLA-II allele separately. SMM Align and Comblib both use a stabilized matrix method. Sturniolo/TEPITOPE used a combination of expert rules and assembled matrices. IEDB Consensus (Concensus3) is an ensemble method that is based on scores from NetMHCIIpan, NN Align, SMM Align and Sturniolo. Depending on the HLA-II allele, IEDB Consensus combines scores from one to three methods to report a ranking score.

### Predicting HLA-DQ Peptide Presentation

A separate model was trained for HLA-DQ2.2-presented peptide that was based on two available HLA-DQ ligandomes for HLA-DQ2.2 and HLA-DQ2.5. The HLA-DQ model neural network architecture is identical to the sequence-only LSTM model for HLA-DR (FIG. 10A), but the model was trained on different datasets (Supplementary FIGS. 30 and 31). The training HLA-DQ2.2 ligandome contains all HLA-DQ ligandome sequences from three DQ2.2 cell lines (PLH 9047, MOU 9050 and PITOUT 9051). HLA-DQ2.5 ligandome sequences (CD114, STEINLIN 9087 and PF04015 9088) were included if the peptide sequences were substrings of any known HLA-DQ2.2 ligandome peptides (FIG. 30). 7.5% of DQ2.2 ligandome was held-out as the validation set to determine model regularization parameters and the training stop point, and another 7.5% to determine the ability of the model to predict human HLA-DQ2.2 peptides (FIG. 31).

The ability of the DQ model was assessed to differentiate DQ2.2-binding wheat gluten peptide (n=69) from length-matched random human peptide (n=138). NetMHCIIpan3.1 was run on the same test set with the allele input of HLA-DQA10201 and HLA-DQB10202. The performance of these two methods was compared by measuring their sensitivity when the specificity was set to 90% (cut-off at the 90th percentile for both MARIA DQ model and NetMHCIIpan3.1; percentile=100%−rank).

### Analysis of Immunoglobulin HLA-DR Presentation Heat Maps

The HLA presentation of Ig from patients with MCL was estimated on the basis of how each amino acid position was covered by experimentally identified or predicted peptide ligands. HLA-presented Ig peptides from 18 MCL samples were obtained from a previous MS-based study. All MS-identified peptides were aligned against a standard Ig template sequence with the Biopython pairwise alignment function. The MS-identified ligand number at each amino acid position is equal to the number of peptide ligands covering the position.

To generate predicted HLA-DR ligands, Ig sequences were broken down from 18 MCL samples into consecutive 15-amino-acid sliding windows. Each sliding window peptide was scored by NetMHCIIpan or MARIA (a version not trained on Ig sequences) with matched patient alleles. High-scoring peptides were aligned against the same Ig template sequence as described above. Scores (S) for peptide sequences were based on their predicted presentation percentiles (p) and the numbers of ligands identified from the corresponding patient samples (q):

$$S ( p , q ) = \{ \begin{matrix}0 , p < 90 th percentile \\ wq ( p - 90 ) , p \geq 90 th percentile\end{matrix}$$

where w is a constant to scale the predicted heat map and MS-identified heat map into a similar scale for plotting purposes. The predicted ligand number at each amino acid position is the sum of scores across 18 MCL samples.
Analyzing Cancer Neoantigen Candidates with MARIA

To score each somatic mutation in two cancer vaccine trials with MARIA, each somatic mutation was represented by a sequence that includes 14 amino acids upstream and downstream of the mutation site, such that the typical sequence length is 29 amino acids (14+1+14). 15-amino-acid sliding windows along these sequences were combined each window with the HLA alleles and gene expression values of each patient to produce a MARIA score. In therapeutic cancer vaccine studies, 50 TPM were used to reflect the high concentration of mutated peptides introduced by vaccines, otherwise median TCGA tissue-matched RNA-seq gene expression was used. The highest score of all sliding windows was used as the score for a given somatic mutation. Neoantigen examples with known unsupported alleles (for example, HLA-DQB1*06:01) were excluded from the analysis. The same analysis was performed with the IEDB CD4 immunogenicity tool for comparison.

### Predictive Performance Metric Calculation

N annotates the number of a group of peptide ligands. Sensitivity (also known as recall) was calculated as:

$$\frac{N ( correctly predicted positive ligands )}{N ( all positive ligands )}$$

Specificity was calculated as:

$$\frac{N ( correctly predicted negative or decoy ligands )}{N ( all negative or decoy ligands )}$$

Positive predictive value (also known as precision) was calculated as:

$$\frac{N ( correctly predicted positive ligands )}{N ( all ligands predicted to be positive )}$$

$$Or$$

$$\frac{Prevalence \times Recall}{Prevalence \times Recall + ( 1 - Prevalence ) \times ( 1 - Specificity )}$$

Negative predictive value was calculated as:

$$\frac{N ( correctly predicted negative or decoy ligands )}{N ( all ligands predicted be negative or decoy )}$$

ROC-AUC scores were calculated on the basis of the area under sensitivity and 1−specificity curves and implemented with Python scikit-learn. To generate precision and recall curves, pairs of recalls and specificities were first calculated across a range of MARIA cut-offs (70th to 99.9th percentile). Precision for each recall was then calculated using an assumption of positive peptide prevalence (1%; Supplementary Note 2).

### Statistical Analysis

Sequence logo plots and amino acid frequency enrichment were generated with IceLogo. Plotting in this study was done in matplotlib and seaborn. Two-tailed paired t tests were used in FIG. 4 for comparing AUC scores with two different methods but on the same set of patient data. Statistical significances difference was determined between two AUC curves (for example, FIG. 20) using the fast DeLong test. Unless otherwise stated, statistically significant differences between distributions were determined by Mann-Whitney U tests. GO term enrichment analysis was conducted with ToppGene. Except GO term enrichment, any statistical P values below 10⁻⁵ were denoted as P<10⁻⁵ or P<1×10⁻⁵.

Supplementary Note 1: Importance of Gene Expression for HLA-II Presentation and Robustness of MARIA with Gene Expression References

In this study, the importance of gene expression or protein abundance for HLA-II presentations was demonstrated. Strikingly, gene expression alone obtained AUC of 0.81 using gene expression as a predictor in the validation set (FIG. 20). However, this strong performance score should be interpreted in the following context.

Genes with high expression or unknown expression were enriched for the decoy genes/peptides in this validation set. When the human genome was sampled with a uniform distribution, AUC of gene expression went up to 0.84 since more non-expressed genes were selected. Conversely, the predictive performance of gene expression values dropped when more highly expressed genes were selected as a negative set (FIG. 14). When the negative set only included genes with TPM>25, gene expression completely lost predictive value.

To understanding whether MARIA requires accurate or even personalized gene expression profiles to predict patients antigen presentations, the following two experiments were performed. For six MCL patients with personalized RNA-Seq data available, the predictive performance of MARIA was evaluated with external RNA-Seq values in comparison to patient matched values. The results yielded a little drop in MARIA performance when using external values (FIG. 15, p=0.32). In the second experiment, the performance of MARIA was compared with tissue mismatched external RNA-Seq values to tissue matched values (SKCM) for identifying HLA-II ligands in whole melanoma tissues. Surprisingly, there was little drop in prediction performance when tissue mismatched gene expression values was used (FIG. 14, 0.895 vs. 0.887-0.889). In both experiments, shuffling gene expression values reduced MARIA performance by fewer than AUC of 0.1, demonstrating the robustness of the model (FIGS. 14 and 15).

For cancer vaccine applications, personalized RNA-Seq profiling for each patient is theoretically desired. However, correlations between CD4 T cells responses and gene expression values of targeted patient neoantigens were not observed (FIG. 42). This is potentially due to that cancer vaccines (either mRNA or peptide vaccines) make antigens largely available to patient antigen presenting cells which is similar to neoantigens overexpressing in tumors. To reflect this mechanism, a fixed TPM value of 50 was used for all vaccine candidates when running MARIA (FIGS. 42 and 45).

### Supplementary Note 2: Estimating HLA-II Ligand Prevalence and Limitations of T-Cell Assays

A 1% prevalence was assumed for presented HLA-II ligands based on the following two CD4 T-cell epitope studies. In a high throughput screen for melanoma neoantigens the researchers identified 4 immunogenic mutated peptides from 458 candidates. In a melanoma vaccine trial, the researchers identified 18 immunogenic mutated peptides from 97 candidates. Both studies were systematic in their approach to screening, and neither of these studies filtered candidates through use of predicted HLA-II binding scores.

HLA-II ligand presentation is essential for CD4 T-cell responses. However, peripheral tolerance, regulatory T-cells, tumor immune editing and low sensitivity of assays can all lead to negative T-cell responses for presented antigens. Further, presented HLA-II neoantigens sometimes stimulate regulatory T-cells rather than conventional CD4 T-cells, which led to negative read-out for common interferon gamma markers. Thus these two studies (0.8% and 19%) suggest a lower bound of the presented ligands for HLA-II. To be on the conservative side, 1% was chosen. This number is higher than directly observed ligand numbers from some MS HLA-ligand studies likely due to limited sensitivities of the current MS technology. A more comprehensive understanding of the fraction of presented HLA-II antigens that are able to elicit strong CD4 T-cell responses will likely require prospective screening of a very large number of antigens in many subjects in future clinical trials.

### Supplementary Note 3: Performance of MARIA and NetMHCIIpan for Differentiating Point Mutations on CLIP

Docking method: Docking of CLIP variants was accomplished using the FlexPepDock, which estimated how peptides interact with a protein complex given structure files (e.g. PDB files) of protein (e.g. HLA-DR1) and peptides (e.g. CLIP). Using PyMOL mutagenesis function to introduce single amino acid mutation in the wild type structure of HLA-DR1 with bound CLIP14 (PDB ID: 3PDO), the conformation with the least strain was selected as the FlexPepDock input file. Structure changes of each mutant complexes were determined based on top three output poses from FlexPepDock.

Docking results: It was tested whether MARIA scores could predict expected changes in binding affinity for single amino acid mutations in peptide ligands as relevant to neoantigens and minor antigens. This is important to test because such mutant peptides were rare in most of validation studies. NetMHCIIpan and MARIA were evaluated when predicting binding/presentation of CLIP (fragment of CD74, PVSKMRMATPLLMQALP) of HLA-DRB1*01:01 complex and its mutated counterparts (FIG. 40). CLIP was chosen because it is the intrinsic ligand necessary for almost all HLA-DR complex formation and its co-crystal structure with HLA-DRB1*01:01 is available (PDB ID: 3PDO).

Using in silico docking experiments and previous structural studies, 7 mutations that enhance, impede, or have little effect on binding were considered. To allow comparison between methods, both raw MARIA and NetMHCIIpan scores were normalized into percentiles by comparing raw scores to random human peptides, where higher percentiles reflect better predicted binding. NetMHCIIpan yielded binding percentiles exceeding 99.85% for all CLIP mutants with the exception of a single CLIP mutant showing a modestly decreased NetMHCIIpan score (R108D, 97.50%). In comparison, MARIA scores for these same peptide mutants were more consistent with the expected structural changes (75.51-97.96%, FIG. 40). For example, M107W, a well-known stabilizing CLIP mutation, increased MARIA percentiles by ˜6% (from 91.60% to 97.96%). This is likely due to enhanced van der Waals interactions created by this mutation, as supported by the crystal structure and independent in vitro biochemical assays. Therefore, MARIA scores capture the impact of single amino acid changes to HLA-II peptide ligands and reflect the corresponding variation in binding affinity.

Supplementary Note 4: Identifying HLA-DR with Mass Spectrometry

HLA-DR immunopeptidome purification: HLA class-DR molecules were isolated and the associated peptides extracted. In brief, cells were lysed for 20 min on ice in 20 mM Tris-HCl (pH 8), 150 mM NaCl, 1% CHAPS, 0.2 mM PMSF, 1× Halt Protease and Phosphatase Inhibitor Cocktail (Thermo Fisher Scientific) supplemented with complete protease inhibitor cocktail (Roche). The lysate was centrifuged (2×30 min, 13,200 rpm at 4° C.) and the resulting supernatant was precleared for 30 min using recombinant Protein A Sepharose fast-flow beads (GE Healthcare). Precleared lysate was incubated with the HLA-DR specific antibody L24317 coupled to rProtein A Sepharose fast-flow beads for 5 h at 4° C. Following the immune-capture of HLA-II molecules, beads were washed with TBS (pH 7.4) and peptides were eluted from the purified HLA molecules using 10% acetic acid. The eluate was then passed through a 10 kDa MWCO size filter and stored at −80° C. until LC-MS/MS analysis.

Mass spectrometry analysis of HLA-DR-associated peptides: Isolated HLA peptides were reconstituted in 12 μl of 0.1% formic acid and analyzed on a Fusion Lumos mass spectrometer (Thermo Fisher Scientific). Peptides were separated by capillary reverse phase chromatography on a 24 cm reversed phase column (100 μm inner diameter, packed in-house with ReproSil-Pur C18-AQ 3.0 m resin). The Fusion Lumos was equipped with a Dionex Ultimate 3000 LC-system and used a two-step linear gradient with 4-25% buffer B (0.1% (v/v) formic acid in acetonitrile) for 80 min followed by 25-45% buffer B for 10 min. Data were acquired in top speed data dependent mode with a duty cycle time of 3 s. Full MS scans were acquired in the Orbitrap mass analyzer with a resolution of 120 000 (FWHM) and m/z scan range of 340-1540. Precursor ions with mass range of 700-2760 and charge state 2-6 and intensity threshold above 50,000 were selected for fragmentation using higher-energy collisional dissociation (HCD) with quadrupole isolation, isolation window of 1.6 m/z and normalized collision energy of 30%. HCD fragments were analyzed in the Orbitrap mass analyzer with a resolution of 15,000 (FWHM). Fragmented ions were dynamically excluded from further selection for a period of 30 seconds. Each sample was measured twice, once with above described HCD method and a second analysis using a method which toggled HCD and electron transfer dissociation (ETD) fragmentation modes for each isolated precursor using the following parameters for ETD: charge state 2 was excluded, calibrated charge dependent ETD parameters were enabled and 25% of supplemental collision energy was used. The AGC target was set to 400000 and 50000 for full FTMS scans and FTMS2 scans. The maximum injection time was set to 50 ms and 200 ms for full FTMS scans and FTMS2 scans.

Computational identification of immunopeptidomes from mass spectra: All tandem mass (MS/MS) spectra were processed using Proteome Discoverer (v 2.2.0.388) and queried against a “target-decoy” sequence database19 consisting of the human UniProt proteome (June 2016) with added common contaminants (e.g. keratins and staphylococcus protein A) using the SEQUEST search engine. SEQUEST search parameters were set as follows: spectrum matching was set to one for b and y ions for HCD and c and z for EThcD. Parent mass error tolerance was set to 10 ppm and fragment mass error tolerance to 0.02 Da. Enzyme specificity was set to none, peptide length was set to 7-25 amino acids, and oxidation of methionines and deamidation (N,Q), cysteinylation, and phosphorylation (S, T, Y) were considered as variable modification. High-confidence peptide identifications were selected at a 1% false discovery rate (FDR) using the Percolator algorithm with a validation based on the q-value. During machine learning, modified amino acid residues were encoded as the original amino acids.

### Supplementary Note 5: Overview of Neural Networks and Implementation

Neural networks or artificial neural networks mimic natural neural networks by constructing multiple layers of gating neuron units to enable a signal processing model with high complexity and flexibility. Neural networks have achieved many successes across a diverse set of classification problems and are fundamental building blocks of deep learning. For a binary classification model, the output layer contains two neurons representing the probability of the input being positive (T) or negative (F) (FIG. 10A). categorical cross entropy was used as the loss function and softmax as the output neuron activation function. Categorical cross entropy is defined as:

Σᵢ₌₁ⁿp(xᵢ)log(q(xᵢ))

where n is the total sample size, p(xᵢ) is the true label of the input xᵢ (0 or 1) and q(xᵢ) is the predicted presentation score for input xᵢ (0-1). Presentation score of an input sequence was defined by the value of positive output neuron (T). The model was trained to minimize the loss function. Softmax functions enable the positive and negative output neuron values to sum up to one.

For a regression model (e.g. predicting binding affinities), the output layer contains a single neuron without additional activation function (FIG. 10B). Mean squared error was used as the loss function for regression model, which measures the difference between experimentally measured values and model predicted values.

A recurrent neural network is a special type of neural network layer designed to process variable length-sequence data (FIG. 5). The recurrent layer in this work is specifically implemented as a Long Short Term Memory (LSTM) neural network as described by Hochreiter and Schmidhuber (S. Hochreiter and J. Schmidhuber, Neural computation 9, 1735-1780 (1997), the disclosure of which is incorporated herein by reference). LSTM has been shown to avoid gradient vanishing and perform better than a normal recurrent neural network. For LSTM, hyperbolic tangent was used as activation functions and hard sigmoid as recurrent activation functions. Binary cross categorical entropy function was used as the loss function and RMSprop as the gradient decent optimizer. Detailed neural network architectures are discussed herein and illustrated in the FIG. 18 and FIGS. 10A and 10B.

Supplementary Note 6: Predicting HLA-II Ligands with Sequence Information Only

With deep RNN/LSTM: A deep neural network model was built solely based on peptide sequences recovered from HLA-DR MS. The model takes in variable length of peptide amino acid sequences (8-26 AA long) and encodes them with one-hot encoding as described (FIG. 10A). A masking layer helps the model ignore all zero vectors (padding) at the end of input matrix when the query peptide is shorter than 26 AAs. The model includes an LSTM layer followed by two dense layers and outputs a probability score (0-1, score of the positive output neuron T). Training and validation data of this model are identical to the full MARIA model dataset. With the same neural network architecture (FIG. 10A), a pan-HLA-II prediction model was trained with peptide sequence information only using a pan-HLA-II ligand dataset.

With shallow neural network (hidden layer=1): The prediction performance of LSTM was compared with a shallow network consisting only of fully connected hidden layers such as NetMHCIIpan. A version of NetMHCII was trained on the same training and validation data as the sequence only LSTM model. Briefly, NetMHCII feeds the following information into a conventional dense neural network: peptide length, peptide N-terminal amino acid, peptide C-terminal amino acid, 9 amino acid sequence (9mer) of predicted binding core, sequence length left to the binding core (left peptide flanking region), sequence length right to the binding core (left peptide flanking region). These information are fed into one hidden layer of 40 neurons 27, and the output is two neurons with binary cross categorical entropy as the cost function. To determine the 9mer binding core, all possible 9mer sliding windows for a query peptide (minimum 9AA long) are generated and fed into the neural network independently. The 9mer window with the highest predicted binding score was chosen as the binding core for the query peptide. No HLA-DR allele information was included in training to be consistent with the LSTM sequence-only model.

### Example 2: Deep Learning Enables Accurate Prioritization of CD8+ Epitopes

### Important Features for MHC-I Antigen Presentation

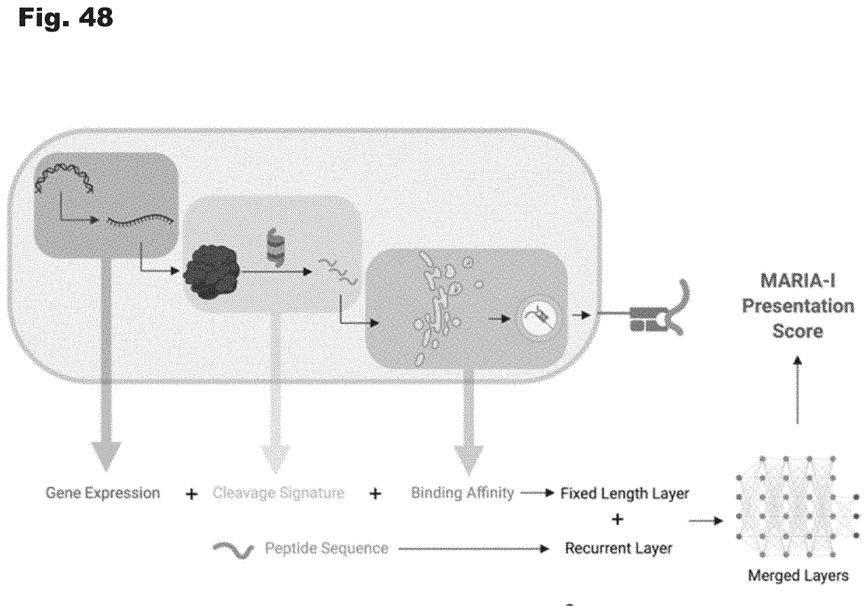

The goal is to build a model that takes into account the biological mechanisms that result in antigen processing and presentation. At the beginning of the antigen presentation pathway, genes are variably expressed and their mRNA products are translated into proteins. To be presented by MHC-I, these proteins must be cleaved by the cytosolic proteasome, carried by the Transporter associated with Antigen Processing (TAP) into the endoplasmic reticulum, and loaded onto the MHC molecule that is transported to the cell membrane for display. In recognition of the selective narrowing of the number of candidate antigens, features were created to reflect the biological patterns within the class I presentation pathway (FIG. 48).

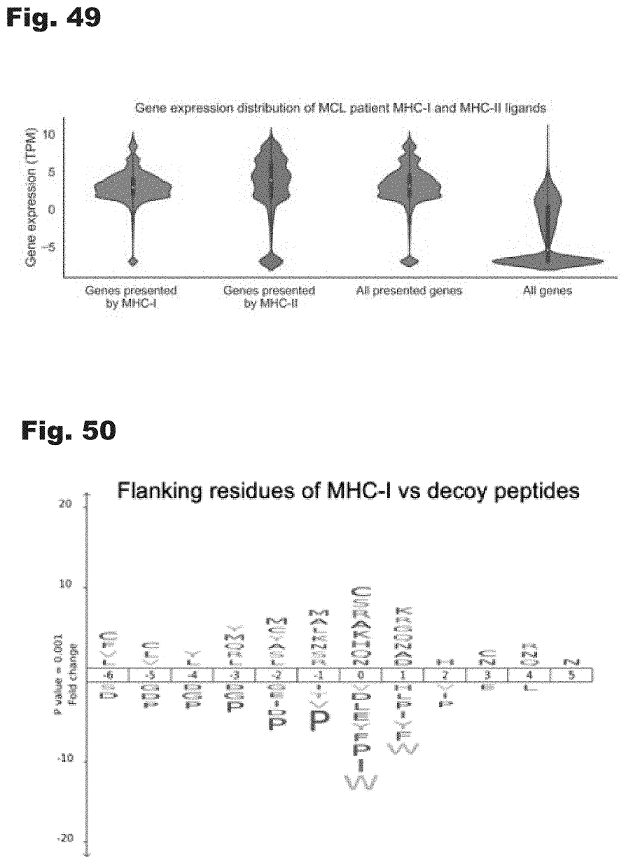

Gene expression influences the availability of protein available for presentation and is significantly correlated with MHC-I antigen presentation. MHC-I presented peptides had lower gene expression compared to MHC-II presented peptides and peptides presented by both MHC-I and MHC-II had higher expression levels compared to non-presented peptides (FIG. 49). Protein cleavage, as mediated by the cytosolic proteasome influences the presented peptidome. A cleavage signature was defined as the six residues upstream and downstream of the N- and C-terminus, respectively. Quantifying cleavage relationships in prior methods like NetChop show promise for better understanding the class I pathway and its effects on peptide specificity. MS-identified peptides from MCL samples were compared to random peptides and uncovered that proline and tryptophan are depleted in upstream and downstream regions, respectively. A diverse set of amino acids, including cysteine, methionine, and tyrosine are enriched in flanking regions (FIG. 50). Prior to the recent advent of large mass-spectrometry peptide datasets, in-vitro binding affinity assays were the favored feature to predict antigen presentation (netMHCpan3.0). MHC-peptide binding is necessary for antigen loading and presentation on the cell surface, representing a continuous filter that narrows the range of displayed peptides. To directly identify peptides presented by MHC-I, MHC-peptide complexes were immunoprecipitated using a pan-MHC-I antibody and eluted peptides were sequenced using MS.

MARIA-I (MHC Analysis with Recurrent Integrated Architecture for Class I) Design

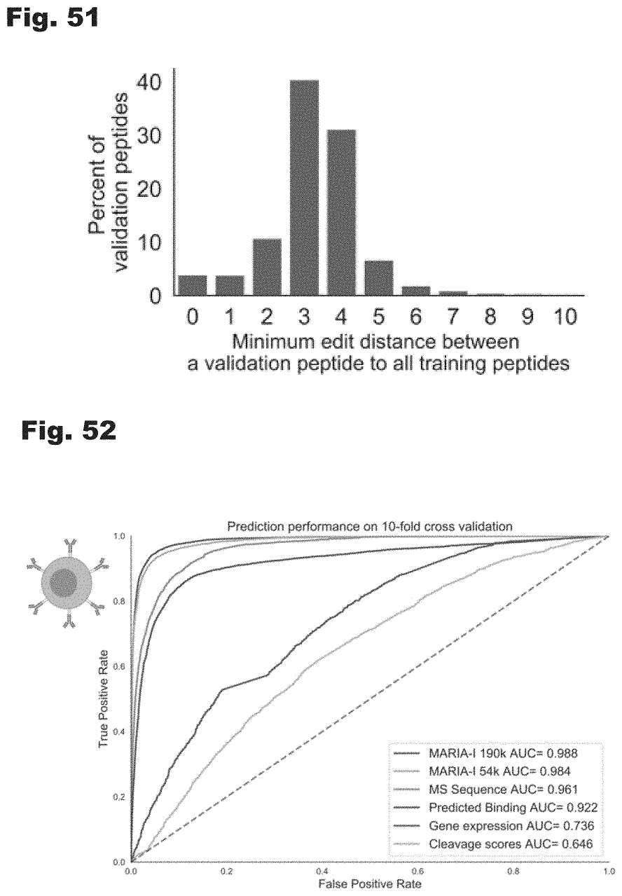

MARIA-I (MHC Analysis with Recurrent Integrated Architecture for class I) combines the features above into a single is a model to predict MHC-I antigen presentation (FIG. 48). MARIA-I incorporates a recurrent neural network (RNN) with long short-term memory (LSTM) to manage varying peptide lengths without needing separate neural networks for each k-mer. Due to this architecture, MARIA-I learns from training data of varying lengths simultaneously and can generate presentation scores for peptides ranging from 8 to 17 amino acids in length. The length distribution of MHC-I peptides is centered on 9-mers, with important contributions 8, 10, and 11-mers, and ranging to 15-mer. MCL MHC-I peptides ranged from 8 to 15 amino acids, with 9-mers representing 64% of all peptides. To prevent overestimation of true performance during internal validation, the training and validation peptides were ensured to be sufficiently unique, with 80% of validation peptides differing from all training peptides by at least 3 amino acids (FIG. 51).

To utilize MARIA-I, the user inputs a query peptide sequence, its corresponding gene name, and the relevant MHC-A, B, and C alleles (6 total). MARIA-I obtains a gene expression value from either a TCGA tissue-matched expression dictionary or user-provided expression values as transcripts per million (TPM). A cleavage sub-model was trained on flanking residues of naturally presented peptides, which uses the gene and peptide to create a cleavage score. The binding affinity sub-model, trained on in-vitro binding data, uses the six alleles and peptide sequence to calculate six binding scores. The query peptide is also encoded in a separate MS-sequence sub-model that is trained to differentiate between naturally presented and decoy peptides based only on amino acid sequences. MARIA-I then merges the gene expression, cleavage, and binding affinity scores with the encoded peptide sequence to generate a presentation score. This presentation score is transformed into a percentile based on a background distribution of 10,000 random human peptides for each MHC allele.

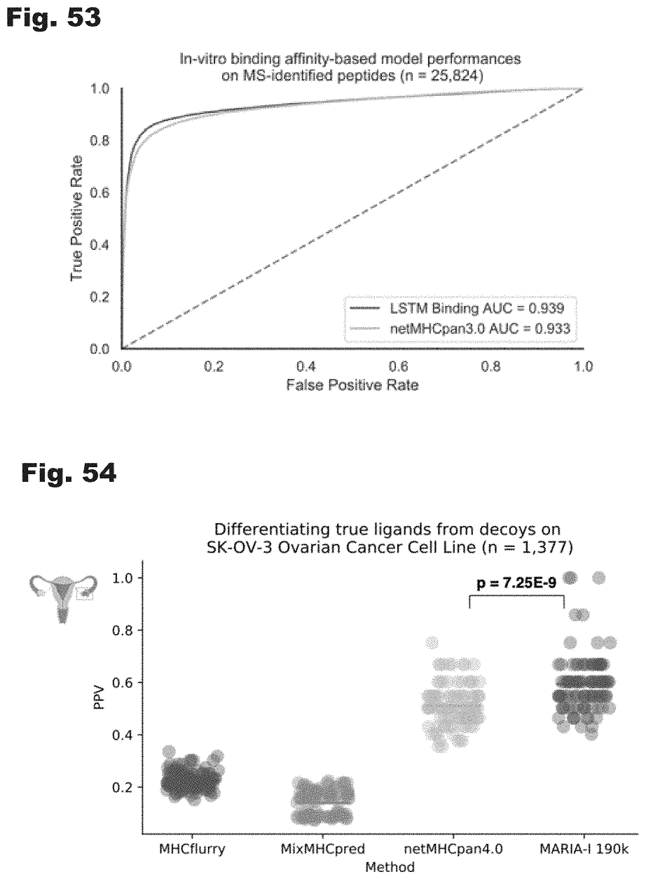

The relative contribution of each feature helps explain its relative importance in predicting antigen presentation. Gene expression had a modest contribution to presentation prediction (FIG. 52, AUC=0.736), contrasted to its more significant predictive power in MHC-II peptide presentation (see Example 1). Cleavage signature performance for MHC-I prediction was comparable to MHC-II, obtaining an AUC of 0.646. Binding affinity had strong predictive value in the MARIA-I model (AUC=0.922), which is comparable to performance by binding affinity-based predictors like netMHCpan3.0 (FIG. 53). However, this method had low precision as peptides with good in-vitro binding properties may not be processed or bind readily to MHC-I in-vivo. Peptide sequence, trained directly on ˜50,000 MS-identified ligands identified from 17 mantle cell lymphomas (MCL), demonstrated the strongest independent sub-model performance on 10-fold cross validation (AUC=0.961). Integration of these four sub-models into MARIA-I outperformed all individual features (AUC=0.984). Addition of ˜140,000 MS-identified peptides from B lymphoblastoid cell lines and melanoma tissue samples to these training data improved internal validation performance, resulting in the final MARIA-I 190 k model (AUC=0.988).

### Benchmarking on Independent Test Data

MARIA-I was assessed and compared with other methods to predict antigen presentation as identified by MS. Other methods for comparison included netMHCpan4.0, MixMHCpred, and MHCflurry, each of which are trained on similar MS antigen data. Three diverse external validation data were utilized for the assessment: 1 ovarian cancer cell line, 12 meningioma samples, and 17 tumors including three chronic lymphocytic leukemia, one ovarian, six glioblastoma, and seven melanoma samples. These three datasets include 209,876 presented MHC-I peptides from a diverse set of MHC-I alleles and 534,431 decoys generated for testing. To truly reflect the rarity of MHC-I neoantigens, a prevalence of 1% was used to calculate positive predictive values (PPV).

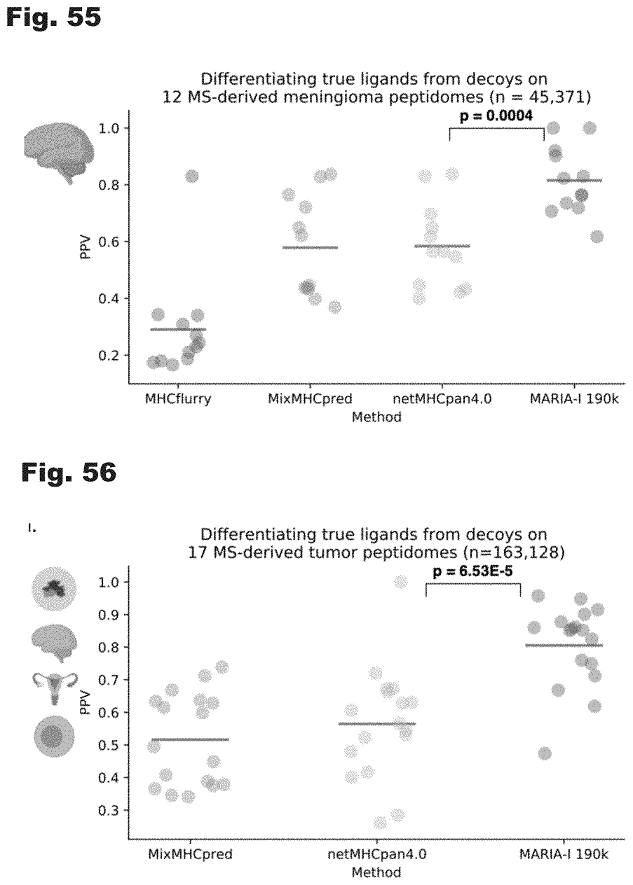

For the ovarian cancer sample, MARIA-I and three alternative methods were applied utilizing 1,377 positive and 4,131 decoy ligands from the MS-identified SK-OV-3 cell line ligands. MARIA-I had a significantly higher PPV compared to netMHCpan4.0, the next best method among other existing methods (PPV 0.594 vs. 0.512, p-value=7.25×10\hat{ }-9, FIG. 54). These models were also assessed on an external MS ligand dataset of 12 meningiomas and noted that MARIA-I outperformed netMHCpan4.0, MixMHCpred, and MHCflurry (PPV=0.82 vs. 0.58, 0.58, and 0.29, respectively, p-value=0.0004 (FIG. 55). To profile MARIA-I's generalizability to multiple tissue types, the models were further assessed on MS-identified peptides from 17 tumors. MARIA-I performance was higher than netMHCpan4.0 and MixMHCpred (PPV=0.81 vs 0.56 and 0.51, respectively, p-value=6.53×10\hat{ }-5 (FIG. 56).

### MHC-1 and MHC-11 Antigen Presentation Algorithm Extended to Murine Alleles

Existing murine tumor models have proved vital insights of tumorigenesis and cancer immunology. Recent studies have indicated needs for MHC antigen prediction tools that can prioritize neoantigens in mouse models. Using MS-identified, publicly available peptides presented on MHC-I haplotype b and MHC-II haplotype I-Ab, Mouse MARIA was developed, an adaptation of the human MARIA models to predict murine antigen presentation. Mouse MARIA models are trained on MS-identified and decoy peptide sequences (11,919 MHC-I ligands and 3,709 MHC-II ligands).

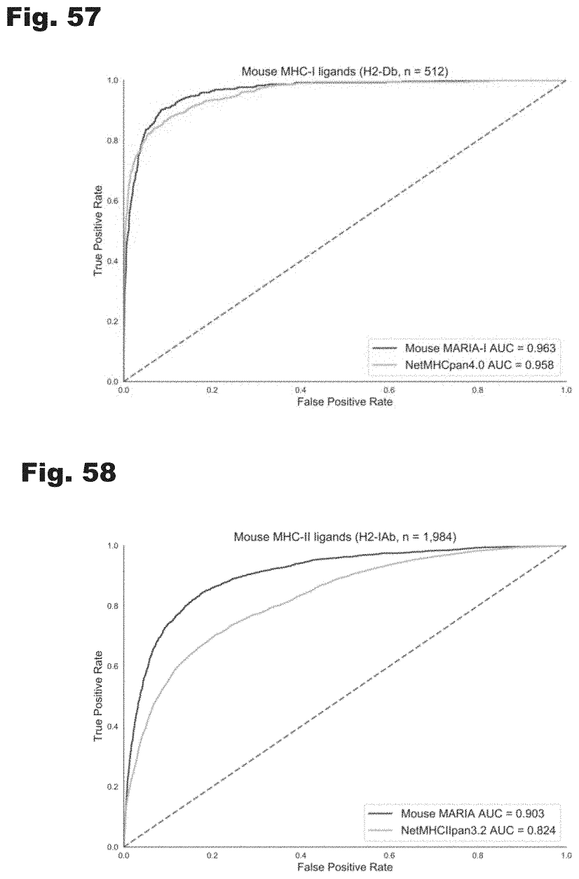

Performance of the MARIA MHC-I haplotype b (genotype) on an external validation set of 512 MS-identified ligands and 5,769 decoys (IEDB) is comparable to netMHCpan4.0 (FIG. 57, AUC=0.963 vs. 0.958, p-value=0.159). MARIA H2-IAb, when evaluated on 1,984 MHC-II ligands and 8,376 decoys, significantly outperforms netMHCIIpan3.2 on MHC-II antigen presentation (FIG. 58, AUC 0.903 vs. 0.824, p-value=2.53E-60).

MARIA-I Scores are Associated with CD8+ T Cell Responses

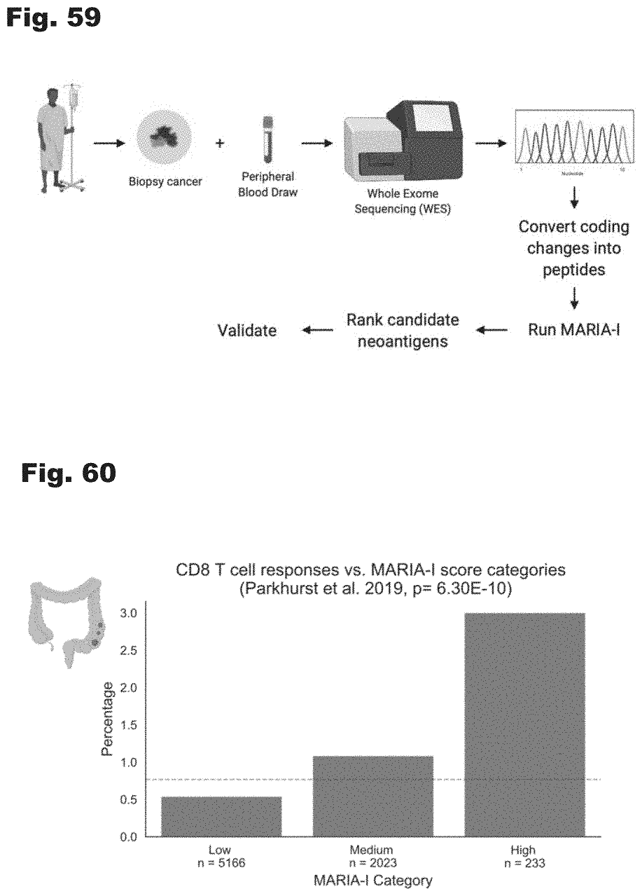

Given the promising results on antigen presentation, MARIA-I was applied to prioritize cancer neoantigens. These peptides are generated by cancer mutations and most likely absent from the normal human genome. They represent a class of promising antigens that may drive anti-tumor T cell responses in immunotherapies. First, a pipeline was created to process publicly available whole exome sequencing (WES) into candidate peptides that can be scored by MARIA-I and other methods (FIG. 59).

MARIA-I was evaluated on a study that tested antigen immunogenicity using in vitro T cell assays on TILs from gastrointestinal cancers (colorectal, bile duct, pancreas, stomach) (n=7,422,). Each candidate antigen derived from exome sequencing was binned into one of three percentile categories (Low: <95, Medium: 95-99.5, High: >99.5). In the absence of immune stimulatory agents, only a small fraction of synthesized neoantigen peptides induced immune responses in their patient TILs (0.74%), but MARIA-I scores helped enrich robust CD8+ T-cell epitopes. Peptides that MARIA-I ranked as high, with scores >99.5 percentile, had a 3% response rate, as compared to 0.5% and 1.1% in the low and medium category, respectively (FIG. 60, P=6.30×10\hat{ }-10).

Cancer vaccines can leverage T-cell mediated cellular immunity through recognition of cancer neoantigens. Clinical trials have shown their promise in anti-tumor immune responses and improved sequencing methods and lowered the barriers to identifying cancer-specific mutations. Identifying strong neoantigens from a pool hundreds of candidates, by their MHC presentation or T cell recognition profiles, is vital for therapeutic success. Previous vaccines designs have prioritized candidate peptides using binding affinity prediction tools like NetMHCpan, which have yielded limited success in generating T cell responses.

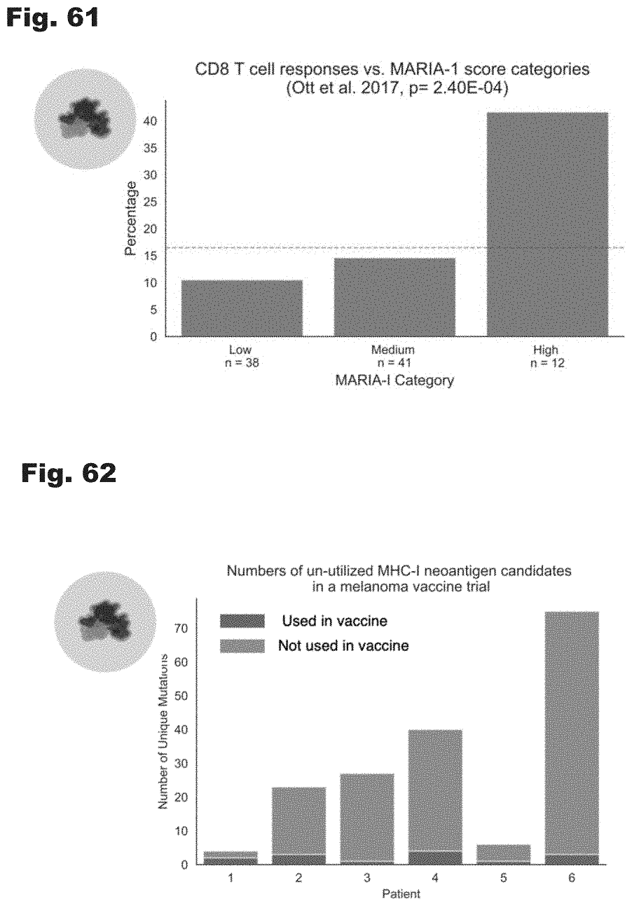

Using the same three percentile cut-offs as above, MARIA-I's ability to select immunogenic neoantigens was evaluated on personalized melanoma vaccine antigens with matched CD8+ T cell response data (ex vivo IFN-gamma release, n=91). 40% of neoantigen peptides in the high category were able to induce a post-vaccination CD8+ T-cell responses measured by ex vivo IFN-γ release assays. In comparison, MARIA-I-ranked antigens in the low and medium bins were less likely to elicit T cell responses (10% and 12%, respectively; FIG. 61, P=2.4×10\hat{ }-4).

To explore whether more promising neoantigens existed within their patient mutation profile, all nonsynonymous SNVs were analyzed as potential neoantigens with MARIA-I from the six patients vaccinated with immunizing peptides. (Fraction of mutations fall into MARIA-I high) On average, 83.8% of mutations labelled as “highly presentable” (>99.5 percentile) by MARIA-I were unused in their vaccine design. Each patient has X-Y promising neoantigen candidates yet utilized by the original study. 16.2% of mutations are labelled as “highly presentable” (>99.5 percentile) by MARIA-I, on average, were used to vaccinate patients with melanoma, suggesting the possibility of missed mutations that may contribute to a strongly presented peptide (FIG. 62).

In summary, MARIA-I scores are significantly correlated with robust CD8+ T-cell responses despite the model being trained on antigen presentation data.

### MARIA-I Results Summary

The prior example in predicting MHC-II antigen presentation showed that combining features derived from the MHC peptide processing pathway, including gene expression, cleavage signature, and binding affinity, with MHC-bound peptides sequenced through MS yielded significant improvements in performance over existing class II predictors (see Example 1). MARIA-I replicated this result in MHC-I antigen presentation. With respect to gene expression, as was the case in MHC-II prediction, tissue-specific levels provide a small improvement over unmatched gene expression levels but allow the model to maintain a high prediction performance despite variations in measurement of this feature.

It is also noted that the majority of published MHC ligand data are from heterozygous samples (not mono-allelic cell lines), in which the 1:1 relationship of peptide to MHC is unknown. This is one of the bottlenecks of training MHC presentation prediction algorithms. NetMHCpan4.0 trains on unambiguous MHC allele data, restricting training data to those peptides that have an identified restricting MHC. MixMHCpred2.0 deconvoluted allele specificity by assigning allele-specific motifs using shared ligands between samples with shared MHC alleles. MARIA-I allows learning from heterozygous samples (e.g. primary tumor samples) to better understand the landscape of physiological MHC presentation beyond mono-allelic cell lines. The results indicate that MARIA-I outperforms existing methods, including netMHCpan4.0, MixMHCpred, and MHCflurry, in diverse tissue types. As MS techniques continue to advance, continued dissemination of peptide presentation data will serve as a cornucopia to improve MHC-I antigen prediction.

Applying MARIA-1 to T-cell epitope prediction resulted in improved presentation performance associated with antigenicity. MARIA-I scores of neoantigens were significantly correlated with their likelihood of eliciting CD8+ T cell responses in ex-vivo assays. However, a key hurdle to building a machine learning algorithm for T-cell epitope prediction is the lack of appropriate negative sets. Appropriate negative training examples are peptides with good presentation properties that do not elicit a T-cell response, which avoids building a model heavily relying on presentation as the main feature.

### MHC-I Ligand Sequence Data

Training data: MCL MHC-A, B, and C ligandomes were retrieved from tumors of 17 patients and two cell lines (JEKO, L128). Publicly available peptide data (n=18,288, 104,966, 47,023), from a diverse set of samples including cell lines derived from B-cell leukemia, basal like breast cancer, and colon carcinoma as well as melanoma tumor tissue, were also included in training.

Independent validation data: SK-OV-3 ovarian cancer cell line ligands (n=1,377). 12 peptidomes identified from meningioma samples were obtained from a previous study (n=45,371,). A recent study profiled 11 tumors with known MHC alleles and exposed 6 of those to additional IFN-gamma treatment, for a total of 17 tumor peptidomes evaluated in this work (n=163,128).

In both training and validation data, peptide:HLA-I complexes were purified using anti-b2M W6/32 antibodies.

### MHC-Typing

Patients' MHC alleles were identified by analyzing tumor exome sequencing of MCL samples through the PHLAT bioinformatics pipeline. Given the wide range of HLA-I alleles and their selective presentation patterns, analyses were restricted to peptide data with known HLA alleles.

### Encoding of Peptide Sequences

Amino acids were coded using “one-hot encoding.” Each residue was represented by a vector of length 21, allowing 20 amino acids and 1 separator. Each vector is a list of zeros and a one in a specific position indicating the identity of the amino acid. This method provides internally consistent map of amino acids and allows peptide sequences to be represented by a matrix of size X,21, with X indicating the number of amino acids in the sequence.

### Gene Expression Data

Gene expression profiles for MCL patients and JeKo-1 cell line were obtained from RNA-sequencing results. The gene expression dictionary for MCL was created by using the median gene expression for each gene across the patients with MCL. Expression values were normalized to TPM for ease of comparison and simplifying user input for MARIA-I.

To ensure the model is robust against gene expression noise and does not rely on patient-specific gene expression profiles, tissue specific was use rather than patient specific RNASeq values (e.g. median TPM across a cohort of MCL patients). For estimated gene expression levels of tumor tissues, the median of TCGA RNA-sequencing results from the closest tissue type were used. For example, melanoma peptides were assigned median TCGA gene expression levels for skin cutaneous melanoma (SKCM). However, for cell lines, available expression data was downloaded from the ENCODE database.

The analyses show minor differences in presentation prediction performance when using tissue-matched gene expression profiles compared to unmatched gene expression levels.

Finally, for the analysis of candidate peptides in vaccine studies, a fixed TPM of 25 was used to ensure consistently high presentation, reflecting the readily available peptides for cells' antigen presentation machinery in a therapeutic context.

### Neural Network Architecture

MARIA-I takes as input a peptide sequence (8-17mer), six patient MHC-A,B,C alleles, and Hugo Symbol gene names. Peptide sequence and MHC alleles are inputs to the binding affinity neural network, and peptide sequence and gene name are inputs to the cleavage score network. Gene names are used to obtain gene expression estimates based on an external TPM dictionary, either specified by the user or defaulting to the MCL dictionary. Peptide sequence is separately one-hot encoded into a 17×21 matrix, with padding and masking to ensure a consistent number of rows despite varying peptide length. The peptide sequence matrix is fed into a special RNN layer called LSTM, which can manage longer associations in sequential data. Normalized outputs from the binding affinity model, cleavage model, and gene expression dictionary are concatenated into a fixed 8×1 vector (6 binding affinities, 1 cleavage score, and 1 gene expression value). The LSTM output is concatenated with the fixed vector of scores of binding, cleavage, and gene expression and inputted into two fully connected dense layers separated by dropout layers. The output of these layers is connected to a softmax activation layer that outputs the likelihood of peptide presentation in the context of predicted binding affinity, cleavage scores, and gene expression level.

The same neural numbers (64 for LSTM, 32 for dense) and drop-out (0.35) were used as in Example 1. Very minor improvements in model performance were observed when doubling the neural number. Categorical cross-entropy is used as the loss function.

### Binding Affinity Prediction

The binding affinity LSTM was trained on publicly available data of 185 k peptides (tools.immuneepitope.org/main/datasets/) curated by the NetMHCpan3.0 research team. IC50 scores were normalized to log scale using the following formula: 1-log 50,000*IC50. Each MHC allele was condensed into a 34-amino-acid sequence, termed a pseudosequence, representing key residues that mediate the MHC-peptide interaction. This interaction is one-hot encoded into a 52×21 matrix, with 52 as the maximum length of the combined pseudosequence (34 amino acids) and peptide (maximum of 17 amino acids). This matrix is fed into an LSTM layer followed by 2 dense layers (neuron number=32) and an output neuron. Mean squared error is used as the loss function and the output is the predicted binding affinity for a given allele-peptide pair.

### Cleavage Score Prediction

This feature provides information about cleavage patterns around the peptide and its relationship to presentation. This pattern was defined as 6 amino acids upstream and downstream of the peptide. To create negative labeled data, length- and gene-matched random peptides were derived from the positive peptide. The cleavage data was one-hot encoded into a 12,21 matrix, with 12 corresponding to the combined number of upstream and downstream amino acids. This matrix was used as input into the cleavage score neural network, which processed the input and used a sigmoid activation function to output the probability that a peptide with the given flanking regions would be presented.

### Percentile Normalization

The MARIA-I softmax activation output provides an expected likelihood of presentation on a 0 to 1 scale. These values are not human-interpretable as they provide little context for the overall distribution of peptide presentation scores. Thus, in addition to the raw score, a percentile score was created to rank the presentation likelihood of a given sample amongst a background distribution of peptides. 10,000 random human peptides were generated for each kmer from 8 to 17, spanning the breadth of variation in peptide length in the MS training data, and calculated MARIA-I scores for each set of 10,000 with a random selection of 6 MHC alleles.

### Generating Random Peptides

To create a random peptide, a gene is selected from a list of 26,013 genes with a probability distribution corresponding to the expression level across genes. Next, a random amino acid position is picked as the first amino acid to generate a peptide with the appropriate length. To prevent the generating random peptides that appear like identified positive peptides, the new peptide is not identical to any known MHC-I presented peptides.

### Benchmarking MARIA-I and Other MS-Based Prediction Methods on Held-Out Ligand Data

To judge the generalizability of MARIA-I's performance, datasets of independent ligands were obtained, processed under a variety of MS methods in different research groups, and tested against existing methods for predicting class I antigen presentation. Peptides below 8 amino acids and above 17 amino acids were excluded from all analyses due to the model's constraints, eliminating a small minority of peptides. Random length-matched human peptides were generated for negative data.

When evaluating independent peptides using MARIA-I and NetMHCpan4.0, MixMHCpred2.0, and MHCflurry, all peptides below 8 amino acids and above 14 amino acids were excluded due to model constraints.

For NetMHCpan4.0, six predictions were generated for each peptide, one for each MHC allele, and the allele with the lowest rank (highest likelihood of presentation) was chosen as the predictor's output for the likelihood of peptide presentation.

MixMHCpred2.0 automatically runs all allele predictions for each peptide. For each sample's peptides, all peptides and all six MHC alleles were input. In the output file, for each peptide, the “%Rank_bestAllele” was selected as MixMHCpred2.0's likelihood of presentation.

Similarly, MHCflurry runs all allele predictions for each peptide. All peptides and MHC alleles were input and, for each peptide, the “mhcflurry_prediction_percentile” column value was selected as the MHCflurry's likelihood of presentation. MHCflurry was excluded from the analysis of the held-out 17 tumor peptidomes due to lack of support for several MHC alleles.

Analysis of T Cell Responses with MARIA-I Scores

Each somatic mutation tested in the two T cell response studies was represented as a 17mer sequence, sampling 8 amino acids upstream and downstream of the mutated residue. Within that sequence, all 9-amino-acid sliding windows were generated from each 17mer, and produced corresponding MARIA-I scores. 25 TPM was used to reflect the high concentration present in a pulsed peptide assay. Each mutation was assigned the highest MARIA-I score calculated from its sub-sampled peptides (a 9mer window). Each peptide was then assigned a presentation category, low, medium, or high based on its maximum MARIA-I percentile score: less than 95th percentile, 95th-99.5th percentile, or above 99.5th percentile.

Receiver Operating Characteristic-Area Under the Curve (ROC-AUC) of a sensitivity vs. 1-specificity graph were implemented with Python scikit-learn.

Positive predictive value (PPV), or precision, was calculated as:

(Prevalence*Recall)/[(Prevalence*Recall+(1−Prevalence)*(1−Specificity)]

Most plots were generated using python packages, matplotlib and seaborn. Motif analysis plots, represented as amino acid frequencies, were generated using IceLogo.

Unless otherwise noted, p-values for differences in statistical distributions were calculated using Student's t-test. For FIGS. 60 and 61, chi-squared test was used to evaluate the expected vs. observed numbers of peptides with positive CD8+ T cell activation. A bootstrapping method, sampling 500 positive and 1,500 decoys peptides each iteration for 100 iterations, was used to generate p-values in analyzing performance of the different methods on the SK-OV-3 cell line.

Sequence Listing Table

| SEQ. | | |
|---|---|---|
| ID | | Associated |
| No. | Sequence | FIGS. |
| 1 | KIFRQMDTNNDGK | 5, 6 |
| 2 | YFDSVPTSRE | 5, 6 |
| 3 | HPHALLVYPTLPEAL | 5, 6 |
| 4 | KEFYLFPTVFDEN | 5, 6 |
| 5 | YGEAELERM | 5, 6, 7 |
| 6 | SYIRTSFDK | 5, 6, 7 |
| 7 | IYYADVKEPE | 5, 6 |
| 8 | YFDSVPTSRECVG | 5 |
| 9 | VKMKNLEKELH | 5, 6 |
| 10 | ALAALSRQEINMEDEE | 5 |
| 11 | YIDSVPTSRECVG | 6 |
| 12 | ALALSRQEINMEDEE | 6 |
| 13 | PKYVKQNTLKLAT | 7 |
| 14 | NTQGLIPP | 7 |
| 15 | GNHAMKPINDNKEP | 7 |
| 16 | VSKMRMATPLLMQA | 7 |
| 17 | STERCVYGPPDSV | 7 |
| 18 | KEELERQAVDQIK | 7 |
| 19 | KVNFFRMVISNPAATHQDID | 7 |
| 20 | ERANSVTWNPHKMMGVPL | 7 |
| 21 | LAVLECLQDVREPENE | 7 |
| 22 | AEMVIHHQHVQDCDE | 7 |
| 23 | FGQAAAGDKPSLF | 7 |
| 24 | KGMAALPRLIAFTSEHSHFS | 7 |
| 25 | RLGRATRKTSERS | 7 |
| 26 | VSDYISELYNKPLYE | 35 |
| 27 | DPDADAIARG | 35 |
| 28 | SLFKNVRLLK | 35 |
| 29 | IYYQLAGYILT | 35 |
| 30 | DTYRSYYADWYQQKPG | 35 |
| 31 | QSNNYAASSYSLTPE | 35 |
| 32 | SNNYAASSYSLTPEQ | 35 |
| 33 | SNNYAASSYSLTPE | 35 |
| 34 | NNYAASSYSLTPE | 35 |
| 35 | LQLVSQFQTVADYA | 35 |
| 36 | LQLVSQFQTVADY | 35 |
| 37 | VPDHVVWSLFNTL | 35 |
| 38 | GGFMTTAFQYIIDNKG | 35 |
| 39 | VYGIFYATSFLDLYRNP | 35 |
| 40 | DKKETVWHLE | 35 |
| 41 | SDVEGFRAVTELG | 35 |
| 42 | EKKYFAATQFEPLAARL | 35 |
| 43 | KKYFAATQFEPLAARL | 35 |
| 44 | EKKYFAATQFEPL | 35 |
| 45 | GPGAPADVQYDLYNVANRR | 35 |
| 46 | NSLRAEDTAVYYGARSPGSSEDYFDYWGQG | 39 |
| 47 | LQMNSLRAEDTAVYYCARASRITIFGVVRK | 39 |
| 48 | CARASRITIFGVVRKSRGGYGMDVWGQGTT | 39 |
| 49 | RVTISVDTSKNQFSLELNSVTAADTAVYYC | 39 |

### DOCTRINE OF EQUIVALENTS

While the above description contains many specific embodiments, these should not be construed as limitations on the scope of the invention, but rather as an example of one embodiment thereof. Accordingly, the scope of the invention should be determined not by the embodiments illustrated, but by the appended claims and their equivalents.

What is claimed is:

1. A method to determine the likelihood that a peptide is presented on a human leukocyte antigen (HLA) receptor of a major histocompatibility complex (MHC), the method comprising:
obtaining one or more peptide sequences for query, wherein each queried peptide has a length between 8 and 26 amino acids;
obtaining a trained peptide presentation module incorporating a recurrent neural network architecture, wherein the peptide presentation module is capable of determining presentation of peptides having varying length to at least one HLA allele;
querying the one or more peptide sequences utilizing the trained peptide presentation module to assess the likelihood to be presented on the at least one HLA allele;
based on the peptide sequence and the at least one HLA allele assessed, determining a MHC presentation score for each peptide of the one or more peptide sequences.

2. The method as in claim 1, wherein the peptide presentation module is trained utilizing in vivo data derived from human individuals or cell lines that have had their MHC peptide ligand sequences identified by antigen presentation profiling via mass spectrometry.

3. The method as in claim 1, wherein the peptide presentation module's recurrent neural network has one of the following architectures: fully recurrent, long short-term memory, gated recurrent unit, bidirectional LSTM or hierarchical recurrent network.

4. The method as in claim 1, wherein at least a first peptide sequence and a second peptide sequence are obtained, wherein each of the peptide length of the first peptide is different from the length of the second peptide.

5. The method as in claim 1, further comprising:
obtaining a trained binding affinity module incorporating a recurrent neural network architecture, wherein the binding affinity module is capable of determining binding affinity of peptides having varying length to a particular HLA allele, and wherein the trained binding affinity module is integrated with the trained peptide presentation module;
querying the one or more peptide sequences utilizing the trained binding affinity module to determine a binding affinity score between each peptide of the one or more peptide sequences and the at least one HLA allele assessed;
based on the peptide sequence, the at least one HLA allele assessed, and the binding affinity score, determining a MHC presentation score for each peptide of the one or more peptide sequences.

6. The method as in claim 5, wherein the binding affinity module is trained utilizing in vitro data derived from the Immune Epitope Database.

7. The method as in claim 5, wherein the binding affinity module's recurrent neural network has one of the following architectures: fully recurrent, long short-term memory, gated recurrent unit, bidirectional LSTM or hierarchical recurrent network.

8. The method as in claim 1, further comprising:
determining the flanking amino acid sequences upstream and downstream for each peptide of the one or more peptide sequences;
obtaining a trained cleavability module incorporating a neural network architecture, wherein the trained cleavability module is capable of determining the cleavability of peptides based on their flanking amino acids, and wherein the trained cleavability module is integrated with the trained peptide presentation module;
querying the one or more peptide sequences utilizing the trained cleavability module to determine a cleavability score for each peptide of the one or more peptide sequences;
based on the peptide sequence, the at least one HLA allele assessed, and the cleavability score, determining a MHC presentation score for each peptide of the one or more peptide sequences.

9. The method as in claim 8, wherein the flanking amino acids are determined from a proteome database.

10. The method as in claim 8, wherein the cleavability module is trained utilizing a ligandome of an antigen presenting cell line.

11. The method as in claim 1 further comprising:
obtaining the gene information for each peptide of the one or more peptide sequences;
obtaining a gene expression module incorporating a neural network architecture, wherein the gene expression module is capable of determining the relative gene expression of peptides based on their gene information, and wherein the gene expression module is integrated with the trained peptide presentation module;
querying the one or more peptide sequences utilizing the trained gene expression module to determine the relative expression level for each peptide of the one or more peptide sequences;
based on the peptide sequence, the at least one HLA allele assessed, and the relative gene expression, determining a MHC presentation score for each peptide of the one or more peptide sequences.

12. The method as in claim 11, wherein the gene expression module determines relative gene expression empirically from personalized RNA sequencing data.

13. The method as in claim 11, wherein the gene expression module determines relative gene expression inferentially from external RNA sequencing data.

14. The method as in claim 1, wherein the gene expression module corrects for low gene expression of extracellular proteins or blood proteins constituents.

15. The method as in claim 1, wherein the MHC presentation score is for MHC I, wherein the binding affinity module is capable of determining binding affinity of peptides having a length between 8 and 17 amino acids, and wherein the at least one HLA allele is an allele of one of: HLA-A, HLA-B, and HLA-C.

16. The method as in claim 15, wherein the at least one HLA allele is all alleles of HLA-A, HLA-B, and HLA-C.

17. The method as in claim 1, wherein the MHC presentation score is for MHC II, wherein the binding affinity module is capable of determining binding affinity of peptides having a length between 8 and 26 amino acids, and wherein the at least one HLA allele is an allele of one of: HLA-DP, HLA-DQ, and HLA-DR.

18. The method as in claim 17, wherein the at least one HLA allele is all alleles of HLA-DP, HLA-DQ, and HLA-DR.

19. The method as in claim 1, wherein the MHC presentation score is a basis for utilizing at least one peptide of the one or more peptide sequences in a downstream application.

20. The method of claim 19, wherein the downstream application is one of:
synthesizing the at least one peptide;
developing a vaccine for cancer or an infectious pathogen utilizing the at least one peptide;
developing a treatment to induce tolerance to the at least one peptide, wherein the peptide is involved with an autoimmune or allergic response; or
developing a T cell therapy to treat cancer based on the at least one peptide.


SEQUENCE LISTING (49 sequences)

SEQ ID NO: 1 - protein, 13 aa, Homo sapiens
KIFRQMDTNNDGK

SEQ ID NO: 2 - protein, 10 aa, Homo sapiens
YFDSVPTSRE

SEQ ID NO: 3 - protein, 15 aa, Homo sapiens
HPHALLVYPTLPEAL

SEQ ID NO: 4 - protein, 13 aa, Homo sapiens
KEFYLFPTVFDEN

SEQ ID NO: 5 - protein, 9 aa, Homo sapiens
YGEAELERM

SEQ ID NO: 6 - protein, 9 aa, Homo sapiens
SYIRTSFDK

SEQ ID NO: 7 - protein, 10 aa, Homo sapiens
IYYADVKEPE

SEQ ID NO: 8 - protein, 13 aa, Homo sapiens
YFDSVPTSRECVG

SEQ ID NO: 9 - protein, 11 aa, Homo sapiens
VKMKNLEKELH

SEQ ID NO: 10 - protein, 16 aa, Homo sapiens
ALAALSRQEINMEDEE

SEQ ID NO: 11 - protein, 13 aa, Homo sapiens
YIDSVPTSRECVG

SEQ ID NO: 12 - protein, 15 aa, Homo sapiens
ALALSRQEINMEDEE

SEQ ID NO: 13 - protein, 13 aa, Homo sapiens
PKYVKQNTLKLAT

SEQ ID NO: 14 - protein, 8 aa, Homo sapiens
NTQGLIPP

SEQ ID NO: 15 - protein, 14 aa, Homo sapiens
GNHAMKPINDNKEP

SEQ ID NO: 16 - protein, 14 aa, Homo sapiens
VSKMRMATPLLMQA

SEQ ID NO: 17 - protein, 13 aa, Homo sapiens
STERCVYGPPDSV

SEQ ID NO: 18 - protein, 13 aa, Homo sapiens
KEELERQAVDQIK

SEQ ID NO: 19 - protein, 20 aa, Homo sapiens
KVNFFRMVISNPAATHQDID

SEQ ID NO: 20 - protein, 18 aa, Homo sapiens
ERANSVTWNPHKMMGVPL

SEQ ID NO: 21 - protein, 16 aa, Homo sapiens
LAVLECLQDVREPENE

SEQ ID NO: 22 - protein, 15 aa, Homo sapiens
AEMVIHHQHVQDCDE

SEQ ID NO: 23 - protein, 13 aa, Homo sapiens
FGQAAAGDKPSLF

SEQ ID NO: 24 - protein, 20 aa, Homo sapiens
KGMAALPRLIAFTSEHSHFS

SEQ ID NO: 25 - protein, 13 aa, Homo sapiens
RLGRATRKTSERS

SEQ ID NO: 26 - protein, 15 aa, Homo sapiens
VSDYISELYNKPLYE

SEQ ID NO: 27 - protein, 10 aa, Homo sapiens
DPDADAIARG

SEQ ID NO: 28 - protein, 10 aa, Homo sapiens
SLFKNVRLLK

SEQ ID NO: 29 - protein, 11 aa, Homo sapiens
IYYQLAGYILT

SEQ ID NO: 30 - protein, 16 aa, Homo sapiens
DTYRSYYADWYQQKPG

SEQ ID NO: 31 - protein, 15 aa, Homo sapiens
QSNNYAASSYSLTPE

SEQ ID NO: 32 - protein, 15 aa, Homo sapiens
SNNYAASSYSLTPEQ

SEQ ID NO: 33 - protein, 14 aa, Homo sapiens
SNNYAASSYSLTPE

SEQ ID NO: 34 - protein, 13 aa, Homo sapiens
NNYAASSYSLTPE

SEQ ID NO: 35 - protein, 14 aa, Homo sapiens
LQLVSQFQTVADYA

SEQ ID NO: 36 - protein, 13 aa, Homo sapiens
LQLVSQFQTVADY

SEQ ID NO: 37 - protein, 13 aa, Homo sapiens
VPDHVVWSLFNTL

SEQ ID NO: 38 - protein, 16 aa, Homo sapiens
GGFMTTAFQYIIDNKG

SEQ ID NO: 39 - protein, 17 aa, Homo sapiens
VYGIFYATSFLDLYRNP

SEQ ID NO: 40 - protein, 10 aa, Homo sapiens
DKKETVWHLE

SEQ ID NO: 41 - protein, 13 aa, Homo sapiens
SDVEGFRAVTELG

SEQ ID NO: 42 - protein, 17 aa, Homo sapiens [sequence omitted]

SEQ ID NO: 43 - protein, 16 aa, Homo sapiens
KKYFAATQFEPLAARL

SEQ ID NO: 44 - protein, 13 aa, Homo sapiens
EKKYFAATQFEPL

SEQ ID NO: 45 - protein, 19 aa, Homo sapiens
GPGAPADVQYDLYNVANRR

SEQ ID NO: 46 - protein, 30 aa, Homo sapiens
NSLRAEDTAVYYGARSPGSSEDYFDYWGQG

SEQ ID NO: 47 - protein, 30 aa, Homo sapiens
LQMNSLRAEDTAVYYCARASRITIFGVVRK

SEQ ID NO: 48 - protein, 30 aa, Homo sapiens
CARASRITIFGVVRKSRGGYGMDVWGQGTT

SEQ ID NO: 49 - protein, 30 aa, Homo sapiens
RVTISVDTSKNQFSLELNSVTAADTAVYYC